SEQUENCE LISTING (89 sequences)

SEQ ID NO: 1 - protein, 120 aa, synthetic construct
  REGION 1..120: note = Synthetic: 5D7 VHH
QVTLRESGASLSLTCAASGRTFASAAMGWFRQAPGEEREFVAAISRSDDTYYADSVKGRFTISRDNAKNTVYLQMNSLKSEDTAVYYCAAVVPTYGNNIYVHSAAYNYWGQGTQVTVKPG

SEQ ID NO: 2 - protein, 126 aa, synthetic construct
  REGION 1..126: note = Synthetic: hz5D7v1 VHH
EVQLVESGGGEVQPGGSLRLSCAASGRTFASAAMGWFRQAPGKGREFVAAISRSDDTYYAESVKGRFTISRDNAKNTLYLQMSSLRAEDTAVYYCAAVVPTYGNNIYVHSAAYNYWGQGTLVTVKP

SEQ ID NO: 3 - protein, 126 aa, synthetic construct
  REGION 1..126: note = Synthetic: hz5D7v2 VHH
EVQLVESGGGEVQPGGSLRLSCAASGRTFASAAMGWFRQAPGKEREFVAAISRSDDTYYAESVKGRFTISRDNAKNTLYLQMSSLRAEDTAVYYCAAVVPTYGNNIYVHSAAYNYWGQGTLVTVKP

SEQ ID NO: 4 - protein, 126 aa, synthetic construct
  REGION 1..126: note = Synthetic: hz5D7v3 VHH
EVQLVESGGGEVQPGGSLRLSCAASGRTFASAAMGWFRQAPGKEREFVAAISRSDDTYYAESVKGRFTISRDNAKNTVYLQMSSLRAEDTAVYYCAAVVPTYGNNIYVHSAAYNYWGQGTLVTVKP

SEQ ID NO: 5 - protein, 126 aa, synthetic construct
  REGION 1..126: note = Synthetic: hz5D7v4 VHH
EVQLVESGGGEVQPGGSLRLSCAASGRTFASAAMGWFRQAPGKEREFVAAISRSDDTYYADSVKGRFTISRDNAKNTVYLQMSSLRAEDTAVYYCAAVVPTYGNNIYVHSAAYNYWGQGTLVTVKP

SEQ ID NO: 6 - protein, 126 aa, synthetic construct
  REGION 1..126: note = Synthetic: hz5D7v7 VHH
EVQLVESGGGEVQPGGSLRLSCAASGRTFASAAMGWFRQAPGKEREFVAAISRSGDTYYADSVKGRFTISRDNAKNTVYLQMSSLRAEDTAVYYCAAVVPTYGNNIYVHSAAYNYWGQGTLVTVKP

SEQ ID NO: 7 - protein, 127 aa, synthetic construct
  REGION 1..127: note = Synthetic: hz5D7v8 VHH
EVQLVESGGGEVQPGGSLRLSCAASGRTFASAAMGWFRQAPGKEREFVAAISRSVGDTYYADSVKGRFTISRDNAKNTVYLQMSSLRAEDTAVYYCAAVVPTYGNNIYVHSAAYNYWGQGTLVTVKP

SEQ ID NO: 8 - protein, 127 aa, synthetic construct
  REGION 1..127: note = Synthetic: hz5D7v9 VHH
EVQLVESGGGEVQPGGSLRLSCAASGRTFASAAMGWFRQAPGKEREFVAAISRSVDDTYYADSVKGRFTISRDNAKNTVYLQMSSLRAEDTAVYYCAAVVPTYGNNIYVHSAAYNYWGQGTLVTVKP

SEQ ID NO: 9 - protein, 119 aa, synthetic construct
  REGION 1..119: note = Synthetic: hz5D7v10 VHH
EVQLVESGGSLRLSCAASGRTFASAAMGWFRQAPGKEREFVAAISRSDDTYYADSVKGRFTISRDNAKNTVYLQMSSLRAEDTAVYYCAAVVPTYGNNIYVHSAAYNYWGQGTLVTVKP

SEQ ID NO: 10 - protein, 126 aa, synthetic construct
  REGION 1..126: note = Synthetic: hz5D7v11 VHH
EVQLVESGGGEVQPGGSLRLSCAASGRTFASAAMGWFRQAPGKEREFVAAISRSDDTYYADSVKGRFTISRDNAKNTVYLQMSSLRAEDTAVYYCAAVVPTYGSNIYVYSAAYNYWGQGTLVTVKP

SEQ ID NO: 11 - protein, 126 aa, synthetic construct
  REGION 1..126: note = Synthetic: hz5D7v12 VHH
EVQLVESGGGEVQPGGSLRLSCAASGRTFASAAMGWFRQAPGKEREFVAAISRSDDTYYAESVKGRFTISRDNAKNTVYLQMSSLRAEDTAVYYCAAVVPTYGSNIYVHSAAYNYWGQGTLVTVKP

SEQ ID NO: 12 - protein, 126 aa, synthetic construct
  REGION 1..126: note = Synthetic: hz5D7v13 VHH
EVQLVESGGGEVQPGGSLRLSCAASGRTFASAAMGWFRQAPGKEREFVAAISRSDDTYYAESVKGRFTISRDNAKNTVYLQMSSLRAEDTAVYYCAAVVPTYGNTIYVHSAAYNYWGQGTLVTVKP

SEQ ID NO: 13 - protein, 126 aa, synthetic construct
  REGION 1..126: note = Synthetic: hz5D7v14 VHH
EVQLVESGGGEVQPGGSLRLSCAASGRTFASAAMGWFRQAPGKEREFVAAISRSDDTYYAESVKGRFTISRDNAKNTVYLQMSSLRAEDTAVYYCAAVVPTYGSSIYVHSAAYNYWGQGTLVTVKP

SEQ ID NO: 14 - protein, 126 aa, synthetic construct
  REGION 1..126: note = Synthetic: hz5D7v15 VHH
EVQLVESGGGEVQPGGSLRLSCAASGRTFASAAMGWFRQAPGKEREFVAAISRSDDTYYAESVKGRFTISRDNAKNTVYLQMSSLRAEDTAVYYCAAVVPTYGNAIYVHSAAYNYWGQGTLVTVKP

SEQ ID NO: 15 - protein, 126 aa, synthetic construct
  REGION 1..126: note = Synthetic: hz5D7v17 VHH
EVQLVESGGGEVQPGGSLRLSCAASGRTFASAAMGWFRQAPGKEREFVAAISRSDDTYYAESVKGRFTISRDNAKNTVYLQMSSLRAEDTAVYYCAAVVPTYGSNIYVHSAAYNYWGQGTLVTVRP

SEQ ID NO: 16 - protein, 126 aa, synthetic construct
  REGION 1..126: note = Synthetic: hz5D7v12L VHH
EVQLLESGGGEVQPGGSLRLSCAASGRTFASAAMGWFRQAPGKEREFVAAISRSDDTYYAESVKGRFTISRDNAKNTVYLQMSSLRAEDTAVYYCAAVVPTYGSNIYVHSAAYNYWGQGTLVTVKP

SEQ ID NO: 17 - protein, 10 aa, synthetic construct
  REGION 1..10: note = Synthetic: CDR1 of 5D7, hz5D7v1, hz5D7v2, hz5D7v3, hz5D7v4, hz5D7v7, hz5D7v8, hz5D7v9, hz5D7v10, hz5D7v11, hz5D7v12, hz5D7v13, hz5D7v14, hz5D7v15, hz5D7v12L, and hz5D7v17
GRTFASAAMG

SEQ ID NO: 18 - protein, 9 aa, synthetic construct
  REGION 1..9: note = Synthetic: CDR2 of 5D7, hz5D7v1, hz5D7v2, hz5D7v3, hz5D7v4, hz5D7v10, hz5D7v11, hz5D7v12, hz5D7v13, hz5D7v14, hz5D7v15, hz5D7v12L, and hzD7v17
AISRSDDTY

SEQ ID NO: 19 - protein, 9 aa, synthetic construct
  REGION 1..9: note = Synthetic: CDR2 of hz5D7v7
AISRSGDTY

SEQ ID NO: 20 - protein, 10 aa, synthetic construct
  REGION 1..10: note = Synthetic: CDR2 of hz5D7v8
AISRSVGDTY

SEQ ID NO: 21 - protein, 10 aa, synthetic construct
  REGION 1..10: note = Synthetic: CDR2 of hz5D7v9
AISRSVDDTY

SEQ ID NO: 22 - protein, 18 aa, synthetic construct
  REGION 1..18: note = Synthetic: CDR3 of 5D7, hz5D7v1, hz5D7v2, hz5D7v3, hz5D7v4, hz5D7v7, hz5D7v8, hz5D7v9, and hz5D7v10
VVPTYGNNIYVHSAAYNY

SEQ ID NO: 23 - protein, 18 aa, synthetic construct
  REGION 1..18: note = Synthetic: CDR3 of hz5D7v11
VVPTYGSNIYVYSAAYNY

SEQ ID NO: 24 - protein, 18 aa, synthetic construct
  REGION 1..18: note = Synthetic: CDR3 of hz5D7v12, hz5D7v12L, and hz5D7v17
VVPTYGSNIYVHSAAYNY

SEQ ID NO: 25 - protein, 18 aa, synthetic construct
  REGION 1..18: note = Synthetic: CDR3 of hz5D7v13
VVPTYGNTIYVHSAAYNY

SEQ ID NO: 26 - protein, 18 aa, synthetic construct
  REGION 1..18: note = Synthetic: CDR3 of hz5D7v14
VVPTYGSSIYVHSAAYNY

SEQ ID NO: 27 - protein, 18 aa, synthetic construct
  REGION 1..18: note = Synthetic: CDR3 of hz5D7v15
VVPTYGNAIYVHSAAYNY

SEQ ID NO: 28 - skipped

SEQ ID NO: 29 - protein, 304 aa, Homo sapiens
  REGION 1..304: note = misc_feature - Human NKp46
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 30 - protein, 133 aa, Homo sapiens [sequence omitted]

SEQ ID NO: 31 - protein, 133 aa, synthetic construct
  REGION 1..133: note = Synthetic: IL-2-T3A H16A E61R P65R D84Y C125S
APASSSTKKTQLQLEALLLDLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQSIISTLT

SEQ ID NO: 32 - protein, 133 aa, synthetic construct
  REGION 1..133: note = Synthetic: IL-2-T3A-H16A-P65R-D84S-C125S
APASSSTKKTQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 33 - protein, 491 aa, synthetic construct
  REGION 1..491: note = Synthetic: hz5D7v12-Fc xELL-Knob-IL2-T3A-H16A-E61R-P65R-D84Y-C125S
EVQLVESGGGEVQPGGSLRLSCAASGRTFASAAMGWFRQAPGKEREFVAAISRSDDTYYAESVKGRFTISRDNAKNTVYLQMSSLRAEDTAVYYCAAVVPTYGSNIYVHSAAYNYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPASSSTKKTQLQLEALLLDLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQSIISTLT

SEQ ID NO: 34 - protein, 353 aa, synthetic construct
  REGION 1..353: note = Synthetic: hz5D7v12-Fc xELL-Hole
EVQLVESGGGEVQPGGSLRLSCAASGRTFASAAMGWFRQAPGKEREFVAAISRSDDTYYAESVKGRFTISRDNAKNTVYLQMSSLRAEDTAVYYCAAVVPTYGSNIYVHSAAYNYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPG

SEQ ID NO: 35 - protein, 353 aa, synthetic construct
  REGION 1..353: note = Synthetic: hz5D7v12-Fc xELL-Knob
EVQLVESGGGEVQPGGSLRLSCAASGRTFASAAMGWFRQAPGKEREFVAAISRSDDTYYAESVKGRFTISRDNAKNTVYLQMSSLRAEDTAVYYCAAVVPTYGSNIYVHSAAYNYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 353 aa, synthetic construct; identical to SEQ ID NO: 34
  REGION 1..353: note = Synthetic: hz5D7v12-Fc xELL-Hole

SEQ ID NO: 37 - protein, 482 aa, synthetic construct
  REGION 1..482: note = Synthetic: Non-targeted VHH-Fc xELL-Knob-IL2-T3A H16A E61R P65R D84Y C125S
EVQLVESGGGEVQPGGSLRLSCAASGSINSINVMEWYRQAPGKERDLVAGITSDGDTNYAESVKGRFTISRDNAKNTLYLQMSSLRAEDTAVYYCRARDWGSLTDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPASSSTKKTQLQLEALLLDLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQSIISTLT

SEQ ID NO: 38 - protein, 345 aa, synthetic construct
  REGION 1..345: note = Synthetic: Non-targeted VHH-Fc xELL-Hole
EVQLVESGGGEVQPGGSLRLSCAASGSINSINVMEWYRQAPGKERDLVAGITSDGDTNYAESVKGRFTISRDNAKNTLYLQMSSLRAEDTAVYYCRARDWGSLTDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 39 - skipped

SEQ ID NO: 40 - protein, 354 aa, synthetic construct
  REGION 1..354: note = Synthetic: hz5D7v12-Fc xELL-Hole
EVQLVESGGGEVQPGGSLRLSCAASGRTFASAAMGWFRQAPGKEREFVAAISRSDDTYYAESVKGRFTISRDNAKNTVYLQMSSLRAEDTAVYYCAAVVPTYGSNIYVHSAAYNYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 41 - protein, 491 aa, synthetic construct
  REGION 1..491: note = Synthetic: hz5D7v17-Fc xELL-Knob-IL2-T3A-H16A-E61R-P65R-D84Y-C125S
EVQLVESGGGEVQPGGSLRLSCAASGRTFASAAMGWFRQAPGKEREFVAAISRSDDTYYAESVKGRFTISRDNAKNTVYLQMSSLRAEDTAVYYCAAVVPTYGSNIYVHSAAYNYWGQGTLVTVRPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPASSSTKKTQLQLEALLLDLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQSIISTLT

SEQ ID NO: 42 - protein, 354 aa, synthetic construct
  REGION 1..354: note = Synthetic: hz5D7v17-Fc xELL-Hole
EVQLVESGGGEVQPGGSLRLSCAASGRTFASAAMGWFRQAPGKEREFVAAISRSDDTYYAESVKGRFTISRDNAKNTVYLQMSSLRAEDTAVYYCAAVVPTYGSNIYVHSAAYNYWGQGTLVTVRPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 43 - protein, 353 aa, synthetic construct
  REGION 1..353: note = Synthetic: hz5D7v17-Fc xELL-Hole
EVQLVESGGGEVQPGGSLRLSCAASGRTFASAAMGWFRQAPGKEREFVAAISRSDDTYYAESVKGRFTISRDNAKNTVYLQMSSLRAEDTAVYYCAAVVPTYGSNIYVHSAAYNYWGQGTLVTVRPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPG

SEQ ID NO: 44 - protein, 224 aa, synthetic construct
  REGION 1..224: note = Synthetic: Fc region xELL H435R, T366S, L368A, Y407V hole
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 45 - protein, 4 aa, synthetic construct
  REGION 1..4: note = Synthetic: Linker
GGGG

SEQ ID NO: 46 - protein, 6 aa, synthetic construct
  REGION 1..6: note = Synthetic: Linker
GGSGGS

SEQ ID NO: 47 - protein, 6 aa, synthetic construct
  REGION 1..6: note = Synthetic: Linker
GGSSGS

SEQ ID NO: 48-52 - skipped (5 entries)

SEQ ID NO: 53 - protein, 213 aa, synthetic construct
  REGION 1..213: note = Synthetic: Fc*
GGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 226 aa, Homo sapiens
  REGION 1..226: note = misc_feature - human IgG1 Fc region
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 223 aa, Homo sapiens
  REGION 1..223: note = misc_feature - human IgG1 xELL Fc region
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 226 aa, synthetic construct
  REGION 1..226: note = Synthetic: Fc region M252Y and M428V (YV) S354C T366W knob
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNHYTQKSLSLSPG

SEQ ID NO: 57 - protein, 226 aa, synthetic construct
  REGION 1..226: note = Synthetic: Fc region M252Y, M428V, H435R (YVR) T366S, L368A, Y407V hole
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNRYTQKSLSLSPG

SEQ ID NO: 58 - protein, 223 aa, synthetic construct
  REGION 1..223: note = Synthetic: Fc region xELL H435R
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQKSLSLSPG

SEQ ID NO: 59 - protein, 223 aa, synthetic construct
  REGION 1..223: note = Synthetic: Fc region xELL M252Y and M428V (YV)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNHYTQKSLSLSPG

SEQ ID NO: 60 - protein, 223 aa, synthetic construct
  REGION 1..223: note = Synthetic: Fc region xELL M252Y and M428L (YL)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 61 - protein, 223 aa, synthetic construct
  REGION 1..223: note = Synthetic: Fc region xELL M252Y, M428L, H435R (YLR)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNRYTQKSLSLSPG

SEQ ID NO: 62 - protein, 223 aa, synthetic construct
  REGION 1..223: note = Synthetic: Fc region xELL M252Y, M428V, H435R (YVR)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNRYTQKSLSLSPG

SEQ ID NO: 63 - protein, 223 aa, synthetic construct
  REGION 1..223: note = Synthetic: Fc region xELL S354C T366W knob
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 223 aa, synthetic construct
  REGION 1..223: note = Synthetic: Fc region xELL H435R S354C T366W knob
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRYTQKSLSLSPG

SEQ ID NO: 65 - protein, 223 aa, synthetic construct
  REGION 1..223: note = Synthetic: Fc region xELL M252Y and M428V (YV) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNHYTQKSLSLSPG

SEQ ID NO: 66 - protein, 223 aa, synthetic construct
  REGION 1..223: note = Synthetic: Fc region xELL M252Y and M428L (YL) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNHYTQKSLSLSPG

SEQ ID NO: 67 - protein, 223 aa, synthetic construct
  REGION 1..223: note = Synthetic: Fc region xELL M252Y, M428L, H435R (YLR) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNRYTQKSLSLSPG

SEQ ID NO: 68 - protein, 223 aa, synthetic construct
  REGION 1..223: note = Synthetic: Fc region xELL M252Y, M428V, H435R (YVR) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNRYTQKSLSLSPG

SEQ ID NO: 69 - protein, 223 aa, synthetic construct
  REGION 1..223: note = Synthetic: Fc region xELL T366S, L368A, Y407V hole
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 70 - protein, 223 aa, synthetic construct
  REGION 1..223: note = Synthetic: Fc region xELL H435R, T366S, L368A, Y407V hole
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPG

SEQ ID NO: 71 - protein, 223 aa, synthetic construct
  REGION 1..223: note = Synthetic: Fc region xELL M252Y and M428V (YV) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNHYTQKSLSLSPG

SEQ ID NO: 72 - protein, 223 aa, synthetic construct
  REGION 1..223: note = Synthetic: Fc region xELL M252Y and M428L (YL) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNHYTQKSLSLSPG

SEQ ID NO: 73 - protein, 223 aa, synthetic construct
  REGION 1..223: note = Synthetic: Fc region xELL M252Y, M428L, H435R (YLR) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNRYTQKSLSLSPG

SEQ ID NO: 74 - protein, 223 aa, synthetic construct
  REGION 1..223: note = Synthetic: Fc region xELL M252Y, M428V, H435R (YVR) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNRYTQKSLSLSPG

SEQ ID NO: 75 - protein, 226 aa, synthetic construct
  REGION 1..226: note = Synthetic: Fc region H435R
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQKSLSLSPG

SEQ ID NO: 76 - protein, 226 aa, synthetic construct
  REGION 1..226: note = Synthetic: Fc region M252Y and M428V (YV)
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNHYTQKSLSLSPG

SEQ ID NO: 77 - protein, 226 aa, synthetic construct
  REGION 1..226: note = Synthetic: Fc region M252Y and M428L (YL)
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 78 - protein, 226 aa, synthetic construct
  REGION 1..226: note = Synthetic: Fc region M252Y, M428L, H435R (YLR)
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNRYTQKSLSLSPG

SEQ ID NO: 79 - protein, 226 aa, synthetic construct
  REGION 1..226: note = Synthetic: Fc region M252Y, M428V, H435R (YVR)
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNRYTQKSLSLSPG

SEQ ID NO: 80 - protein, 226 aa, synthetic construct
  REGION 1..226: note = Synthetic: Fc region S354C T366W knob
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 226 aa, synthetic construct
  REGION 1..226: note = Synthetic: Fc region H435R S354C T366W knob
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRYTQKSLSLSPG

SEQ ID NO: 82 - protein, 226 aa, synthetic construct
  REGION 1..226: note = Synthetic: Fc region M252Y and M428L (YL) S354C T366W knob
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNHYTQKSLSLSPG

SEQ ID NO: 83 - protein, 226 aa, synthetic construct
  REGION 1..226: note = Synthetic: Fc region M252Y, M428L, H435R (YLR) S354C T366W knob
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNRYTQKSLSLSPG

SEQ ID NO: 84 - protein, 226 aa, synthetic construct
  REGION 1..226: note = Synthetic: Fc region M252Y, M428V, H435R (YVR) S354C T366W knob
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNRYTQKSLSLSPG

SEQ ID NO: 85 - protein, 226 aa, synthetic construct
  REGION 1..226: note = Synthetic: Fc region T366S, L368A, Y407V hole
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 86 - protein, 226 aa, synthetic construct
  REGION 1..226: note = Synthetic: Fc region H435R, T366S, L368A, Y407V hole
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPG

SEQ ID NO: 87 - protein, 226 aa, synthetic construct
  REGION 1..226: note = Synthetic: Fc region M252Y and M428V (YV) T366S, L368A, Y407V hole
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNHYTQKSLSLSPG

SEQ ID NO: 88 - protein, 226 aa, synthetic construct
  REGION 1..226: note = Synthetic: Fc region M252Y and M428L (YL) T366S, L368A, Y407V hole
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNHYTQKSLSLSPG

SEQ ID NO: 89 - protein, 226 aa, synthetic construct
  REGION 1..226: note = Synthetic: Fc region M252Y, M428L, H435R (YLR) T366S, L368A, Y407V hole
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNRYTQKSLSLSPG

(19) United States
(12) Patent Application Publication
(10) Pub. No.: US 2024/0376198 A1
(43) Pub. Date: Nov. 14, 2024

(54) NKp46-Targeted Modified IL-2 Polypeptides and Uses Thereof

(71) Applicant: Inhibrx, Inc., La Jolla, CA (US)

(72) Inventors: John C. Timmer, San Diego, CA (US); Brendan P. Eckelman, Encinitas, CA (US); Rajay A. Pandit, San Diego, CA (US); William Crago, San Diego, CA (US); Florian Sulzmaier, San Diego, CA (US); Heather Kinkead, San Diego, CA (US); Nadja Kern, Del Mar, CA (US)

(73) Assignee: Inhibrx, Inc., La Jolla, CA (US)

(21) Appl. No.: 18/685,666

(22) PCT Filed: Aug. 29, 2022
(86) PCT No.: PCT/US22/75583

§ 371 Date: Feb. 22, 2024

Related U.S. Application Data
(60) Provisional application No. 63/296,776, filed on Jan. 5, 2022.
(60) Provisional application No. 63/238,431, filed on Aug. 30, 2021.

(51) Int. Cl. C07K 16/28; A61K 35/768; A61K 38/00; A61K 39/00; A61K 39/395; A61K 47/68; A61P 35/00; C07K 14/55; C07K 16/30

(52) U.S. Cl. CPC C07K 16/2803; A61K 35/768; A61K 39/3955; A61K 39/39558; A61K 39/4611; A61K 39/4631; A61K 47/68031; A61K 47/68033; A61K 47/68035; A61K 47/6809; A61K 47/6849; A61K 47/6851; A61P 35/00; C07K 14/55; C07K 16/2818; C07K 16/2827; C07K 16/30; A61K 38/00; C07K 2317/24; C07K 2317/31; C07K 2317/35; C07K 2317/569; C07K 2319/30

(57) ABSTRACT

Provided herein are NKp46-targeted modified IL-2 polypeptides. Uses of the polypeptides are also provided.

### CROSS-REFERENCE TO RELATED APPLICATIONS

This application claims the benefit of priority of U.S. Provisional Application No. 63/238,431, filed Aug. 30, 2021, and U.S. Provisional Application No. 63/296,776, filed Jan. 5, 2022; each of which is incorporated by reference herein in its entirety for any purpose.

### INCORPORATION BY REFERENCE OF SEQUENCE LISTING

This application incorporates by reference a Sequence Listing submitted with this application in electronic format entitled 01202-0051-00PCT_ST26, created Aug. 18, 2022 which is 118 kilobytes in size.

### FIELD

The present invention relates to NKp46-targeted modified IL-2 polypeptides, and methods of using such polypeptides to modulate the biological activity of NK cells. Such methods include, but are not limited to, methods of treating cancer and infectious diseases. In some embodiments, the NKp46-targeted modified IL-2 polypeptides are fusion polypeptides comprising a NKp46-targeted modified IL-2 polypeptide and a modified IL-2.

### BACKGROUND

NKp46, also known as CD335, LY94-homolog, or NCR1, is an activating cell-surface receptor expressed on natural killer (NK) cells. It is part of the natural cytotoxicity receptor (NCR) family that generally function as receptors for stress ligands displayed on virally infected, fungal, or cancer cells. Ligation and clustering of NKp46 drives activating signals through its immunoreceptor tyrosine-based activation motif (ITAM) containing co-receptors Fc epsilon RI and CD3 zeta, which induce the expression of interferon-gamma and NK-mediated cytotoxicity. NKp46 expression is restricted to NK cells and is not expressed on CD4+ or CD84-T-cells, B-cells, monocytes, or granulocytes. The NKp46 gene is conserved from humans to cynomolgus monkeys, rats, and mice; and the expression pattern in these animals is also restricted to NK cells just as in humans. This expression pattern and species conservation make it an ideal NK-specific marker for a NK-targeted therapeutic, and also a potent NK-activating receptor to drive NK-mediated cytotoxicity.

NK cells are key immune cells that are able to kill virally infected and cancer cells without prior sensitization and enhance the adaptive immune response of dendritic cells, T-cells, and B-cells through cytokine and chemokine signals (Vidal et al Curr Opin Virol 1(6):497-512 (2011)). NK cells have several mechanisms to recognize and kill target cells. Stress ligands engage and activate NK cell degranulation through natural cytotoxicity receptors (NCRs) such as NKG2D, NKp30, NKp44, NKp46, and DNAM-1. MHC class I loss is a common immune evasion strategy of many viruses and is also observed in human cancers. NK cells can recognize and kill cells not expressing MHC class I by withdrawal of inhibitory signals from KIRs and NKG2A (Raulet and Vance Nat Rev Immunol 6(7):520-531 (2006)). Antibody opsonization of viral or cancer antigens by antibodies agonize CD16a on NK cells, inducing potent activation of NK cytotoxicity. NK cell degranulation releases cytotoxic proteins as well as immune stimulating cytokines such as interferon-gamma and TNF-alpha; and immune recruiting chemokines like CCL3, CCL4, CCL5, XCL1, and XCL2 (Fauriat et al. Blood 115(11):2167-2176 (2010) and Bottcher et al. Cell 172(5):1022-1037 (2018)). These secreted factors activate and recruit DCs, T-cells, and B-cells to coordinate an efficient adaptive immune response.

Numerous stimulatory and suppressive signals coordinate the overall activation status of NK cells, and control the threshold and magnitude of response. Ideal conditions for NK mediated killing of virally infected cells requires support from cytokines such as interleukin-2 (IL-2). IL-2 is a potent cytokine that stimulates T and NK cell proliferation through a heterodimeric signaling receptor composed of CD122 and CD132 and through a heterotrimeric high affinity form of the receptor that includes CD122, CD132, and CD25. In addition to enhancing NK cell survival and proliferation, IL-2 can prime NK cells to express effector molecules such as granzyme-B, perforin, and interferon-gamma that are released upon degranulation and act to destroy target cells. Pathological inflammation resulting from infection or cancer can drive NK cells into an exhausted and ineffective state. Stimulation with IL-2 can reinvigorate exhausted NK cells and overcome suppressive immune signals.

Therefore, targeting IL-2 to NK cells via NKp46 may increase the potency and selectivity of the cytotoxic NK cell response, and there exists a need for NKp46-targeted modified IL-2 polypeptides.

### SUMMARY

Provided herein are NKp46-targeted modified IL-2 polypeptides and methods of using NKp46-targeted modified IL-2 polypeptides to treat, for example, cancer or infectious diseases. In some embodiments, a NKp46-targeted modified IL-2 polypeptide comprises at least one VHH domain that binds NKp46 and a modified IL-2. In some embodiments, a NKp46-targeted modified IL-2 polypeptide comprises one or more additional binding domains and/or cytokine sequences.

Some embodiments provided herein are below.

Embodiment 1. A polypeptide comprising at least one VHH domain that binds NKp46 and a modified IL-2, wherein at least one VHH domain that binds NKp46 comprises a CDR1 comprising the amino acid sequence of SEQ ID NO: 17; a CDR2 comprising the amino acid sequence of SEQ ID NO: 18, 19, 20, or 21; and a CDR3 comprising the amino acid sequence of SEQ ID NO: 22, 23, 24, 25, 26, or 27, and wherein the modified IL-2 comprises T3A, H16A, P65R, C125S mutations and a D84S or a D84Y mutation relative to a wild type human IL-2 comprising the amino acid sequence of SEQ ID NO 30.

Embodiment 2. The polypeptide of embodiment 1, wherein the modified IL-2 comprises T3A, H16A, P65R, C125S, and D84S mutations.

Embodiment 3. The polypeptide of embodiment 1, wherein the modified IL-2 comprises T3A, H16A, E61R, P65R, C125S, and D84Y mutations.

Embodiment 4. The polypeptide of embodiment 1 or 2, wherein the modified IL-2 comprises the amino acid sequence of SEQ ID NO: 31.

Embodiment 5. The polypeptide of embodiment 1 or 3, wherein the modified IL-2 comprises the amino acid sequence of SEQ ID NO: 32.

Embodiment 6. The polypeptide of any one of embodiments 1-5, wherein at least one VHH domain comprises a CDR1 comprising the amino acid sequence of SEQ ID NO: 17; a CDR2 comprising the amino acid sequence of SEQ ID NO: 18; and a CDR3 comprising the amino acid sequence of SEQ ID NO: 22.

Embodiment 7. The polypeptide of any one of embodiments 1-5, wherein at least one VHH domain comprises a CDR1 comprising the amino acid sequence of SEQ ID NO: 17; a CDR2 comprising the amino acid sequence of SEQ ID NO: 19; and a CDR3 comprising the amino acid sequence of SEQ ID NO: 22.

Embodiment 8. The polypeptide of any one of embodiments 1-5, wherein at least one VHH domain comprises a CDR1 comprising the amino acid sequence of SEQ ID NO.: 17; a CDR2 comprising the amino acid sequence of SEQ ID NO: 20; and a CDR3 comprising the amino acid sequence of SEQ ID NO: 22.

Embodiment 9. The polypeptide of any one of embodiments 1-5, wherein at least one VHH domain comprises a CDR1 comprising the amino acid sequence of SEQ ID NO: 17; a CDR2 comprising the amino acid sequence of SEQ ID NO: 21; and a CDR3 comprising the amino acid sequence of SEQ ID NO: 22.

Embodiment 10. The polypeptide of any one of embodiments 1-5, wherein at least one VHH domain comprises a CDR1 comprising the amino acid sequence of SEQ ID NO: 17; a CDR2 comprising the amino acid sequence of SEQ ID NO: 18; and a CDR3 comprising the amino acid sequence of SEQ ID NO: 23.

Embodiment 11. The polypeptide of any one of embodiments 1-5, wherein at least one VHH domain comprises a CDR1 comprising the amino acid sequence of SEQ ID NO: 17; a CDR2 comprising the amino acid sequence of SEQ ID NO: 18; and a CDR3 comprising the amino acid sequence of SEQ ID NO: 24.

Embodiment 12. The polypeptide of any one of embodiments 1-5, wherein at least one VHH domain comprises a CDR1 comprising the amino acid sequence of SEQ ID NO: 17; a CDR2 comprising the amino acid sequence of SEQ ID NO: 18; and a CDR3 comprising the amino acid sequence of SEQ ID NO: 25.

Embodiment 13. The polypeptide of any one of embodiments 1-5, wherein at least one VHH domain comprises a CDR1 comprising the amino acid sequence of SEQ ID NO: 17; a CDR2 comprising the amino acid sequence of SEQ ID NO: 18; and a CDR3 comprising the amino acid sequence of SEQ ID NO: 26.

Embodiment 14. The polypeptide of any one of embodiments 1-5, wherein at least one VHH domain comprises a CDR1 comprising the amino acid sequence of SEQ ID NO: 17; a CDR2 comprising the amino acid sequence of SEQ ID NO: 18; and a CDR3 comprising the amino acid sequence of SEQ ID NO: 27.

Embodiment 15. The polypeptide of any one of embodiments 1-14, wherein at least one VHH domain is humanized.

Embodiment 16. The polypeptide of any one of embodiments 1-15, wherein at least one VHH domain comprises an amino acid sequence at least 85%, 90%, 95%, or at least 99% identical to the amino acid sequence of any one of SEQ ID NOs: 1-16.

Embodiment 17. The polypeptide of any one of embodiments 1-15, wherein at least one VHH domain comprises the amino acid sequence of any one of SEQ ID NOs: 1-16.

Embodiment 18. The polypeptide of any one of embodiments 1-17, comprising two VHH domains.

Embodiment 19. The polypeptide of any one of embodiments 1-17, comprising three VHH domains.

Embodiment 20. The polypeptide of any one of embodiments 1-19, wherein the polypeptide comprises at least one binding domain that binds an antigen other than NKp46.

Embodiment 21. The polypeptide of any one of embodiments 1-20, wherein the polypeptide comprises at least one binding domain that binds a tumor antigen.

Embodiment 22. The polypeptide of any one of embodiments 1-21, wherein the polypeptide comprises at least one binding domain that binds an antigen selected from 1-92-LFA-3, 5T4, Alpha-4 integrin, Alpha-V integrin, alpha4beta1 integrin, alpha4beta7 integrin, AGR2, Anti-Lewis-Y, Apelin J receptor, APRIL, B7-H3, B7-14, B7-H6, BAFF, BCMA, BTLA, C5 complement, C-242, CA9, CA19-9, (Lewis a), Carbonic anhydrase 9, CD2, CD3, CD6, CD9, CD11a, CD19, CD20, CD22, CD24, CD25, CD27, CD28, CD30, CD33, CD38, CD39, CD40, CD40L, CD41, CD44, CD44v6, CD47, CD51, CD52, CD56, CD64, CD70, CD71, CD73, CD74, CD80, CD81, CD86, CD95, CD117, CD123, CD125, CD132, (IL-2RG), CD133, CD137, CD138, CD166, CD172A, CD248, CDH6, CEACAM5 (CEA), CEACAM6 (NCA-90), CLAUDIN-3, CLAUDIN-4, cMet, Collagen, Cripto, CSFR, CSFR-1, CTLA-4, CTGF, CXCL10, CXCL13, CXCR1, CXCR2, CXCR4, CYR61, DL44, DLK1, DLL3, DLL4, DPP-4, DSG1, EDA, EDB, EGFR, EGFRviii, Endothelin B receptor (ETBR), ENPP3, EpCAM, EPHA2, EPHB2, ERBB3, F protein of RSV, FAP, FAS, FcRH5, FGF-2, FGF8, FGFR1, FGFR2, FGFR3, FGFR4, FLT-3, Folate receptor alpha (FRα), GAL3ST1, G-CSF, G-CSFR, GD2, GITR, GLUT1, GLUT4, GM-CSF, GM-CSFR, GP IIb/IIIa receptors, Gp130, GPIIB/IIIA, GPNMB, GPRC5D, GRP78, HAVCAR1, HER2/neu, HER3, HER4, HGF, hGH, HVEM, Hyaluronidase, ICOS, IFNalpha, IFNbeta, IFNgamma, IgE, IgE Receptor (FceRI), IGF, IGF1R, IL1B, IL1R, IL2, IL1, IL12, IL12p40, IL-12R, IL-12Rbeta1, IL13, IL13R, IL15, IL17, IL18, IL21, IL23, IL23R, IL27/IL27R (wsx1), IL29, IL-31R, IL31/IL31R, IL2R, IL4, IL4R, IL6, IL6R, Insulin Receptor, Jagged Ligands, Jagged 1, Jagged 2, KISS1-R, LAG-3, LIF-R, Lewis X, LIGHT, LRP4, LRRC26, Ly6G6D, LyPD1, MCSP, Mesothelin, MICA, MICB, MRP4, MUC1, Mucin-16 (MUC16, CA-125), Na/K ATPase, NGF, Nicasuin, NKG2A, Notch Receptors, Notch 1, Notch 2, Notch 3, Notch 4, NOV, OSM-R, OX-40, PAR2, PDGF-AA, PDGF-BB, PDGFRalpha, PDGFRbeta, PD-1, PD-L1, PD-L2, Phosphatidyl-serine, P1GF, PSCA, PSMA, PSGR, RAAG12, RAGE, SLC44A4, Sphingosine 1 Phosphate, STEAP1, STEAP2, TAG-72, TAPA1, TEM-8, TGFbeta, TGFbeta receptor 1 (TGFBR1), TGFbeta receptor 2 (TGFBR2), TIGIT, TIM-3, TLR2, TLR4, TLR6, TLR7, TLR8, TLR9, TMEM31, TNFalpha, TNFR, TNFRS12A, TRAIL-R1, TRAIL-R2, Transferrin, Transferrin receptor, TRK-A, TRK-B, TROP-2 uPAR, VAP1, VCAM-1, VEGF, VEGF-A, VEGF-B, VEGF-C, VEGF-D, VEGFR1, VEGFR2, VEGFR3, VISTA, WISP-1, WISP-2, and WISP-3.

Embodiment 23. The polypeptide of any one of embodiments 1-19, wherein each VHH domain binds NKp46.

Embodiment 24. The polypeptide of embodiment 23, wherein each VHH domain comprises the same CDR1, CDR2, and CDR3 amino acid sequences.

Embodiment 25. The polypeptide of embodiment 24, wherein each VHH domain comprises the same VHH sequence.

Embodiment 26. The polypeptide of any one of embodiments 1-17, comprising one VHH domain.

Embodiment 27. The polypeptide of any one of embodiments 1-26, wherein the NKp46 is human NKp46.

Embodiment 28. The polypeptide of embodiment 27, wherein the human NKp46 comprises the sequence of SEQ ID NO: 29.

Embodiment 29. The polypeptide of any one of embodiments 1-28, wherein the polypeptide comprises an Fc region.

Embodiment 30. The polypeptide of embodiment 29, wherein the Fc region comprises an amino acid sequence selected from SEQ ID NOs: 44 and 53-89.

Embodiment 31. The polypeptide of embodiment 29 or embodiment 30, which forms a dimer under physiological conditions.

Embodiment 32. The polypeptide of any one of embodiments 29-31, wherein the modified IL-2 is fused to the C-terminus of the Fc region.

Embodiment 33. The polypeptide of any one of embodiments 29-32, wherein the polypeptide comprises one VHH domain that binds NKp46, an Fc region, and a modified IL-2.

Embodiment 34. The polypeptide of embodiment 33, wherein the polypeptide comprises the amino acid sequence of SEQ ID NO 33.

Embodiment 35. A complex comprising a first polypeptide and a second polypeptide, wherein the first polypeptide is the polypeptide of any one of embodiments 1-34, wherein the first polypeptide comprises a first Fc region, and wherein the second polypeptide comprises at least one VHH domain and a second Fc region, wherein the first and second Fc regions are the same or different.

Embodiment 36. A complex comprising a first polypeptide and a second polypeptide, wherein the first polypeptide comprises at least one VHH domain that binds NKp46 and a first Fc region and the second polypeptide comprises a second Fc region and a modified IL-2, wherein at least one VHH domain that binds NKp46 comprises a CDR1 comprising the amino acid sequence of SEQ ID NO: 17; a CDR2 comprising the amino acid sequence of SEQ ID NO: 18, 19, 20, or 21; and a CDR3 comprising the amino acid sequence of SEQ ID NO: 22, 23, 24, 25, 26, or 27; and wherein the modified IL-2 comprises T3A or T3G, H16A, P65R, C125S, and D84S or D84Y mutations relative to a wild type human IL-2 comprising the amino acid sequence of SEQ ID NO: 30.

Embodiment 37. The complex of embodiment 36, wherein the modified IL-2 comprises T3A, 116A, P65R, C125S, and D84S mutations.

Embodiment 38. The complex of embodiment 36, wherein the modified IL-2 comprises T3A, 116A, E61R, P65R, C125S, and D84Y mutations.

Embodiment 39. The complex of any one of embodiments 36-38, wherein the modified IL-2 comprises an amino acid sequence that is at least 90%, at least 95%, at least 97%, at least 98%, at least 99%, or 100% identical to the amino acid sequence of SEQ ID NO: 31 or 32.

Embodiment 40. The complex of any one of embodiments 36, 38, and 39, wherein the modified IL-2 comprises the amino acid sequence of SEQ ID NO: 31.

Embodiment 41. The complex of any one of embodiments 36-40, wherein at least one VHH domain, or each VHH domain, is humanized.

Embodiment 42. The complex of any one of embodiment 36-41, wherein at least one VHH domain comprises an amino acid sequence at least 85%, at least 90%, at least 95%, or at least 99% identical to an amino acid sequence selected from SEQ ID NOs: 1-16.

Embodiment 43. The complex of any one of embodiments 36-42, wherein at least one VHH domain comprises an amino acid sequence selected from SEQ ID NOs: 1-16.

Embodiment 44. The complex of any one of embodiments 35-43, wherein the second polypeptide comprises at least one antigen-binding domain.

Embodiment 45. The complex of embodiment 44, wherein at least one antigen-binding domain of the second polypeptide is a VHH domain.

Embodiment 46. The complex of embodiment 45, wherein the second polypeptide comprises at least one VHH domain that binds NKp46.

Embodiment 47. The complex of any one of embodiments 35-46, wherein the second polypeptide comprises at least one VHH domain that binds NKp46.

Embodiment 48. The complex of embodiment 47, wherein the second polypeptide comprises at least one VHH domain that binds NKp46 and comprises a CDR1 comprising the amino acid sequence of SEQ ID NO: 17; a CDR2 comprising the amino acid sequence of SEQ ID NO 18, 19, 20, or 21; and a CDR3 comprising the amino acid sequence of SEQ ID NO: 22, 23, 24, 25, 26, or 27.

Embodiment 49. The complex of embodiment 47 or 48, wherein the second polypeptide comprises at least one VHH domain that binds NKp46 and comprises a CDR1, a CDR2, and a CDR3, respectively comprising the amino acid sequences of SEQ ID NOs: 17, 18, and 22; 17, 19, and 22; 17, 20, and 22; 17, 21, and 22; 17, 18, and 23; 17, 18, and 24; 17, 18, and 25; 17, 18, and 26; or 17, 18, and 27.

Embodiment 50. The complex of any one of embodiments 47-49, wherein the second polypeptide comprises at least one VHH domain that binds NKp46 and comprises a CDR1 comprising the amino acid sequence of SEQ ID NO: 17; a CDR2 comprising the amino acid sequence of SEQ ID NO: 18; and a CDR3 comprising the amino acid sequence of SEQ ID NO: 24.

Embodiment 51. The complex of any one of embodiments 47-50, wherein at least one VHH domain of the second polypeptide binds NKp46 and comprises an amino acid sequence at least 85%, 90%, 95%, or at least 99% identical to the amino acid sequence of SEQ ID NO: 1, 2, 3, 4, 5, 6, 7, 8, 9, 10, 11, 12, 13, 14, 15, or 16.

Embodiment 52. The complex of any one of embodiments 47-51, wherein at least one VHH domain of the second polypeptide binds NKp46 and comprises the amino acid sequence of SEQ ID NO: 1, 2, 3, 4, 5, 6, 7, 8, 9, 10, 11, 12, 13, 14, 15, or 16.

Embodiment 53. The complex of any one of embodiments 47-52, wherein at least one VHH domain of the second polypeptide binds NKp46 and comprises the amino acid sequence of SEQ ID NO: 11 or 15.

Embodiment 54. The complex of any one of embodiments 47-53, wherein the second polypeptide comprises one VHH domain that binds NKp46.

Embodiment 55. The complex of any one of embodiments 35-54, wherein the first or the second polypeptide comprises at least one VHH domain that binds an antigen other than NKp46.

Embodiment 56. The complex of embodiment 55, wherein the first or the second polypeptide comprises at least one binding domain that binds TGFbeta receptor 1, TGFbeta receptor 2, or NKG2A.

Embodiment 57. The complex of embodiment 55 or embodiment 56, wherein the first or the second polypeptide comprises at least one binding domain that binds a tumor antigen.

Embodiment 58. The complex of any one of embodiments 35-57, wherein the first or the second polypeptide comprises at least one binding domain that binds an antigen selected from 1-92-LFA-3, 5T4, Alpha-4 integrin, Alpha-V integrin, alpha4beta1 integrin, alpha4beta7 integrin, AGR2, Anti-Lewis-Y, Apelin J receptor, APRIL, B7-H3, B7-H4, B7-H6, BAFF, BCMA, BTLA, C5 complement, C-242, CA9, CA19-9, (Lewis a), Carbonic anhydrase 9, CD2, CD3, CD6, CD9, CD11a, CD19, CD20, CD22, CD24, CD25, CD27, CD28, CD30, CD33, CD38, CD39, CD40, CD40L, CD41, CD44, CD446, CD47, CD51, CD52, CD56, CD64, CD70, CD71, CD73, CD74, CD80, CD81, CD86, CD95, CD117, CD123, CD125, CD132, (IL-2RG), CD133, CD137, CD138, CD166, CD172A, CD248, CDH6, CEACAM5 (CEA), CEACAM6 (NCA-90), CLAUDIN-3, CLAUDIN-4, cMet, Collagen, Cripto, CSFR, CSFR-A, CTLA-4, CTGF, CXCL10, CXCL13, CXCR1, CXCR2, CXCR4, CYR61, DL44, DLK1, DLL3, DLL4, DPP-4, DSG1, EDA, EDB, EGFR, EGFRviii, Endothelin B receptor (ETBR), ENPP3, EpCAM, EPHA2, EPHB2, ERBB3, F protein of RSV, FAP, FAS, FcRH5, FGF-2, FGF8, FGFR1, FGFR2, FGFR3, FGFR4, FLT-3, Folate receptor alpha (FRα), GAL3ST1, G-CSF, G-CSFR, GD2, GITR, GLUT1, GLUT4, GM-CSF, GM-CSFR, GP IIb/IIIa receptors, Gp130, GPIIB/IIIA, GPNMB, GPRC5D, GRP78, HAVCAR1, HER2/neu, HER3, HER4, HGF, hGH, HVEM, Hyaluronidase, ICOS, IFNalpha, IFNbeta, IFNgamma, IgE, IgE Receptor (FceRI), IGF, IGF1R, IL1B, IL1R, IL2, IL11, IL12, IL12p40, IL-12R, IL-12Rbeta1, IL13, IL13R, IL15, IL17, IL18, IL21, IL23, IL23R, IL27/IL27R (wsx1), IL29, IL-31R, IL31/IL31R, IL2R, IL4, IL4R, IL6, IL6R, Insulin Receptor, Jagged Ligands, Jagged 1, Jagged 2, KISS1-R, LAG-3, LIF-R, Lewis X, LIGHT, LRP4, LRRC26, Ly6G6D, LyPD1, MCSP, Mesothelin, MICA, MICB, MRP4, MUC1, Mucin-16 (MUC16, CA-125), Na/K ATPase, NGF, Nicastrin, Notch Receptors, Notch 1, Notch 2, Notch 3, Notch 4, NOV, OSM-R, OX-40, PAR2, PDGF-AA, PDGF-BB, PDGFRalpha, PDGFRbeta, PD-1, PD-L1, PD-L2, Phosphatidyl-serine, P1GF, PSCA, PSMA, PSGR, RAAG12, RAGE, SLC44A4, Sphingosine 1 Phosphate, STEAP1, STEAP2, TAG-72, TAPA1, TEM-8, TGFbeta, TIGIT, TIM-3, TLR2, TLR4, TLR6, TLR7, TLR8, TLR9, TMEM31, TNFalpha, TNFR, TNFRS12A, TRAIL-R1, TRAIL-R2, Transferrin, Transferrin receptor, TRK-A, TRK-B, TROP-2 uPAR, VAP1, VCAM-1, VEGF, VEGF-A, VEGF-B, VEGF-C, VEGF-D, VEGFR1, VEGFR2, VEGFR3, VISTA, WISP-1, WISP-2, and WISP-3.

Embodiment 59. The complex of any one of embodiments 35-58, wherein at least one VHH domain, or each VHH domain, of the second polypeptide is humanized.

Embodiment 60. The complex of any one of embodiments 35-59, wherein the first Fc region comprises at least one knob mutation and the second Fc region comprises at least one hole mutation; or wherein the first Fc region comprises at least one hole mutation and the second Fc region comprises at least one knob mutation.

Embodiment 61. The complex of embodiment 60, wherein the first or second Fc region comprises a T366W mutation and the other of the first or second Fc region comprises T366S, L368A, and Y407V mutations.

Embodiment 62. The complex of embodiment 61, wherein the Fc region comprising the T366S, L368A, and Y407V mutations further comprises a H435R or H435K mutation.

Embodiment 63. The complex of any one of embodiments 35-62, wherein the first polypeptide comprises the amino acid sequence of SEQ ID NO: 34, 40, 42, or 43.

Embodiment 64. The complex of any one of embodiments 35-63, wherein the second polypeptide comprises the amino acid sequence of SEQ ID NO: 33 or 41.

Embodiment 65. The complex of embodiment 63 or embodiment 64, wherein:
i) the first polypeptide comprises the amino acid sequence of SEQ ID NO: 34 and the second polypeptide comprises the amino acid sequence of SEQ ID NO: 33,
ii) the first polypeptide comprises the amino acid sequence of SEQ ID NO: 40 and the second polypeptide comprises the amino acid sequence of SEQ ID NO: 33;
iii) the first polypeptide comprises the amino acid sequence of SEQ ID NO: 42 and the second polypeptide comprises the amino acid sequence of SEQ ID NO: 41; or
iv) the first polypeptide comprises the amino acid sequence of SEQ ID NO: 43 and the second polypeptide comprises the amino acid sequence of SEQ ID NO: 41.

Embodiment 66. The complex of any one of embodiments 35-65, wherein the complex forms under physiological conditions.

Embodiment 67. An immunoconjugate comprising the polypeptide or complex of any one of embodiments 1-66 and a cytotoxic agent.

Embodiment 68. The immunoconjugate of embodiment 67, wherein the cytotoxic agent is selected from a calicheamicin, an auristatin, a dolastatin, a tubulicin, a maytansinoid, a cryptophycin, a duocarmycin, an esperamicin, a pyrrolobenzodiazepine, and an enediyne antibiotic.

Embodiment 69. The immunoconjugate of embodiment 67 or 68, wherein the immunoconjugate comprises a complex of any one of embodiments 35-66, and wherein the first or the second polypeptide comprises at least one binding domain that binds CD3, T-cell receptor (TCR) α, TCRβ, CD28, CD16, CD32A, CD64, CD89, or NKG2D.

Embodiment 70. A pharmaceutical composition comprising the polypeptide or complex of any one of embodiments 1-66 or the immunoconjugate of any one of embodiments 67-69, and a pharmaceutically acceptable carrier.

Embodiment 71. An isolated nucleic acid that encodes the polypeptide of any one of embodiments 1-34.

Embodiment 72. A vector comprising the nucleic acid of embodiment 71.

Embodiment 73. A host cell comprising the nucleic acid of embodiment 71 or the vector of embodiment 72.

Embodiment 74. A host cell that expresses the polypeptide of any one of embodiments 1-34.

Embodiment 75. A method of producing the polypeptide of any one of embodiments 1-34, comprising incubating the host cell of embodiment 73 or embodiment 74 under conditions suitable for expression of the polypeptide.

Embodiment 76. The method of embodiment 75, further comprising isolating the polypeptide.

Embodiment 77. An isolated nucleic acid that encodes the first polypeptide and the second polypeptide of the complex of any one of embodiments 35-66.

Embodiment 78. A vector that comprises the isolated nucleic acid of embodiment 77.

Embodiment 79. A host cell comprising the nucleic acid of embodiment 77 or the vector of embodiment 78.

Embodiment 80. A host cell that expresses the complex of any one of embodiments 35-66.

Embodiment 81. A method of producing the complex of any one of embodiments 35-66, comprising incubating the host cell of embodiment 73 or embodiment 74 under conditions suitable for expression of the complex.

Embodiment 82. The method of embodiment 81, further comprising isolating the complex.

Embodiment 83. A method of treating cancer comprising administering to a subject with cancer a pharmaceutically effective amount of the polypeptide or complex of any one of embodiments 1-66, the immunoconjugate of any one of embodiments 67-69, or the pharmaceutical composition of embodiment 70.

Embodiment 84. The method of embodiment 83, wherein the cancer is selected from basal cell carcinoma, biliary tract cancer; bladder cancer; bone cancer; brain and central nervous system cancer; breast cancer; cancer of the peritoneum, cervical cancer; choriocarcinoma, colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer, stomach cancer, testicular cancer, thyroid cancer; uterine or endometrial cancer; cancer of the urinary system; vulval cancer; lymphoma; Hodgkin's lymphoma, non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLI); acute lymphoblastic leukemia (ALL); acute myeloid leukemia (AML); Hairy cell leukemia; and chronic myeloblastic leukemia.

Embodiment 85. The method of embodiment 83 or embodiment 84, further comprising administering an additional therapeutic agent.

Embodiment 86. The method of embodiment 85, wherein the additional therapeutic agent is an anti-cancer agent.

Embodiment 87. The method of embodiment 86, wherein the anti-cancer agent is selected from a chemotherapeutic agent, an anti-cancer biologic, radiation therapy, CAR-T therapy, and an oncolytic virus.

Embodiment 88. The method of embodiment 87, wherein the additional therapeutic agent is an anti-cancer biologic.

Embodiment 89. The method of embodiment 88, wherein the anti-cancer biologic is an agent that inhibits PD-1 and/or PD-L1.

Embodiment 90. The method of embodiment 88, wherein the anti-cancer biologic is an agent that inhibits VISTA, gpNMB, B7H3, B7H4, HHLA2, CTLA4, or TIGIT.

Embodiment 91. The method of any one of embodiments 86-90, wherein the anti-cancer agent is an antibody.

Embodiment 92. The method of embodiment 91, wherein the antibody comprises a binding domain that binds a tumor antigen.

Embodiment 93. The method of embodiment 88, wherein the anti-cancer biologic is a cytokine.

Embodiment 94. The method of embodiment 87, wherein the anti-cancer agent is CAR-T therapy.

Embodiment 95. The method of embodiment 87, wherein the anti-cancer agent is an oncolytic virus.

Embodiment 96. The method of any one of embodiments 83-95, further comprising tumor resection and/or radiation therapy.

Embodiment 97. A method of redirecting a natural killer mediated cytotoxic response to a cancer cell comprising administering to a subject with cancer a pharmaceutically effective amount of the polypeptide or complex of any one of embodiments 1-66, the immunoconjugate of any one of embodiments 67-69, or the pharmaceutical composition of embodiment 70.

Embodiment 98. A method of treating an infectious disease comprising administering to a subject with an infectious disease a pharmaceutically effective amount of the polypeptide or complex of any one of embodiments 1-66, the immunoconjugate of any one of embodiments 67-69, or the pharmaceutical composition of embodiment 70.

Embodiment 99. The method of embodiment 98, wherein the infectious disease is a bacterial, viral, or fungal infection.

Embodiment 100 The method of embodiment 98 or 99, further comprising administering an additional therapeutic agent.

Embodiment 101. The method of embodiment 100, wherein the additional therapeutic agent is an antibiotic, an anti-viral agent, or an anti-fungal agent.

Embodiment 102. A method of redirecting a natural killer mediated cytotoxic response to a pathogen comprising administering to a subject with an infectious disease caused by the pathogen a pharmaceutically effective amount of the polypeptide or complex of any one of embodiments 1-66, the immunoconjugate of any one of embodiments 67-69, or the pharmaceutical composition of embodiment 70.

Embodiment 103. The method of any one of embodiments 98-102, wherein the polypeptide, complex, or immunoconjugate comprises at least one binding domain that binds an antigen expressed by the pathogen.

### BRIEF DESCRIPTION OF THE FIGURES

FIG. 1A-1J show binding of polypeptides comprising NKp46-binding VHH domains and Fc domains assessed by flow cytometry. FIGS. 1A-1B and 1I show binding to HEK-293F cells transfected with human NKp46 FIG. 1C-1D show binding to HEK-293F cells transfected with cynomolgus monkey NKp46. FIG. 1E-1F show binding to HEK-293F cells transfected with mouse NKp46. Binding to untransfected HEK-293F cells is shown in FIGS. 1G-1H and 1J. FIG. 1I-1J show the binding of NKp46-targeting VHH domains formatted as polypeptides comprising bivalent VHH and a homodimeric Fc.

FIG. 2A-2I show activities, as measured by intracellular STAT5 phosphorylation levels, of a polypeptide comprising an IL-2 variant fused to the C-terminus of a heterodimeric Fc and a NKp46-targeted VHH domain (cx10454 or cx11314), a polypeptide comprising a heterodimeric Fc and a NKp46-targeted VHH domain with no IL-2 (cx8411), a polypeptide comprising an IL-2 variant fused to the C-terminus of a heterodimeric Fc and a non-targeting VHH (cx9452), and wild type recombinant IL-2 on CD56^{dim}CD16⁺ NK cells (FIG. 2A-2B), CD56^{bright}CD16 NK cells (FIG. 2C-2D), all NK cells (FIG. 2G), CD4⁺ T cells (FIGS. 2E-2F, and 2H), and CD8⁺ T cells (FIG. 2I). Specificity of signaling activity is demonstrated on NK cells (FIGS. 2A-2D, and 2G), while only wild type recombinant IL-2 has activity on CD4⁺ (FIG. 2E-2F, and 2H) or CD8⁺ T cells (FIG. 2I).

FIG. 3 shows the ADCC activities of a polypeptide comprising an IL-2 variant fused to the C-terminus of a heterodimeric Fc and a NKp46-targeted VHH domain (cx10454), a polypeptide comprising an IL-2 variant fused to the C-terminus of a heterodimeric Fc and a non-targeting VHH (cx9452), and wild type recombinant IL-2 in combination with a suboptimal dose of cetuximab (0.2 nM) relative to the activity of an optimal dose of cetuximab (20 nM).

FIG. 4A-4B show the enhancement of NK cell ADCC activity toward the Raji B-cell lymphoma cell line by a polypeptide comprising an IL-2 variant fused to the C-terminus of a heterodimeric Fc and a NKp46-targeted VHH domain (cx11314), when combined with a sequence analog of rituximab, an anti-CD20 antibody, or an afucosylated variant thereof. FIG. 4A shows a titration of the anti-CD20 antibodies, whereas FIG. 4B shows the impact of altering the NK cell to target cell (Raji) ratio in the presence of 1 nM of the anti-CD20 antibodies.

FIG. 5A-5D show the enhancement of NK cell ADCC activity by a polypeptide comprising an IL-2 variant fused to the C-terminus of a heterodimeric Fc and a NKp46-targeted VHH domain (cx11314), toward. NCI-H929 a multiple myeloma cell line (FIG. 5A) when combined with a sequence analog of daratumumab, an anti-CD38 antibody, at 5 nM or an anti-BCMA antibody at 5 nM; Raji a B cell lymphoma cell line (FIG. 5B) when combined with sequence analogs of tafasitamab, an anti-CD19 Fc engineered antibody, at 10 nM or obinutuzumab, an anti-CD19 Fc engineered antibody, at 1 nM; A549 a lung carcinoma cell line (FIG. 5C) or SKBR3 a breast carcinoma cell line (FIG. 5D) when combined with cetuximab, an anti-EGFR antibody, at 5 nM or trastuzumab, the anti-HER2 antibody, at 50 nM. Herein various NK cell to target cell ratios were compared. cx11314 was used at 1 nM in all combination conditions.

FIG. 6A-6C show the activity of a single dose at 0.3 mg/kg, 1 mg/kg, or 3 mg/kg of a polypeptide comprising a NKp46-binding VHH domain, a heterodimeric Fc region, and an IL-2 variant fused to the C-terminus of the Fc region (cx10454) in cynomolgus monkeys. Expansion of subpopulations within the peripheral blood of the animals is shown at 10 days (FIG. 6A) and 14 days (FIG. 613) after dosing. FIG. 6C shows the increase in granzyme B expression 4 and 10 days after dosing.

FIG. 7 shows the anti-tumor efficacy as measured by changes in the tumor volume induced by a polypeptide comprising a NKp46-binding VHH domain, a heterodimeric Fc, and an IL-2 variant fused to the C-terminus of the Fc region (cx11314) in a sub-cutaneous Raji tumor xenograft mouse model. The NKp46-targeted modified IL-2 polypeptide was dosed once a week for three doses as indicated by the arrows and was given intravenously either as a single agent or in combination with a rituximab-analog. Control groups included treatments with only the vehicle or the rituximab-analog alone.

FIG. 8A-8B show the recovery of chemotherapy-induced NK cell defects by a polypeptide comprising a NKp46-binding VHH domain, a heterodimeric Fc, and an IL-2 variant fused to the C-terminus of the Fc region (cx11314). FIG. 8A shows the effects on NK cell counts in human peripheral blood as determined by flow cytometry after a three-day treatment with dexamethasone alone or in combination with lenalidomide and/or cx11314. FIG. 8B shows the ADCC activity of NK cells pre-treated with chemotherapy (dexamethasone and lenalidomide) alone or in combination with cx11314 against a multiple myeloma target cell line (MM1 S) when combined with a sequence analog of daratumumab (anti-hCD38-hIgG1).

### DETAILED DESCRIPTION

Embodiments provided herein relate to NKp46-targeted modified IL-2 polypeptides and their use in various methods of treating, for example, cancer or infectious diseases.

Provided herein are NKp46-targeted modified IL-2 polypeptides. In some embodiments, the NKp46-targeted modified IL-2 polypeptides comprise at least one VHH that binds NKp46. In some embodiments, the VHH that binds NKp46 is linked to a modified IL-2 for NK cell-targeted IL-2 activity. In some embodiments, the NKp46-targeted modified IL-2 polypeptides also bind another antigen. e.g., comprise a VHH domain that binds another antigen. In some such embodiments, the NKp46-targeted modified IL-2 polypeptide is bispecific. This bispecific NKp46-targeted polypeptide may redirect NK-mediated cytotoxicity towards cells that express the other antigen targeted by the polypeptide. In some embodiments, the NKp46-targeted modified IL-2 is a trifunctional polypeptide that binds NKp46 and another antigen, and comprises a modified IL-2. This trifunctional polypeptide may focus IL-2 activity toward NK cells, while the second targeting domain targets the polypeptide to a particular cell type, such as a cancer cell.

### Definitions and Various Embodiments

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N Y CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D G. Newell, eds., 1993-8) J. Wiley and Sons, Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds, 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty, ed., IRL Press, 1988-1989); Monoclonal Antibodies A Practical Approach (P. Shepherd and C. Dean, eds, Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993); and updated versions thereof.

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

In general, the numbering of the residues in an immunoglobulin heavy chain is that of the EU index as in Kabat et cii, Sequences of Proteins of Immunological Interest, 5th Ed Public Health Service, National Institutes of Health, Bethesda, Md. (1991). The “EU index as in Kabat” refers to the residue numbering of the human IgG1 EU antibody.

It is understood that embodiments of the invention described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The phrase “reference sample”, “reference cell”, or “reference tissue”, denote a sample with at least one known characteristic that can be used as a comparison to a sample with at least one unknown characteristic. In some embodiments, a reference sample can be used as a positive or negative indicator. A reference sample can be used to establish a level of protein and/or mRNA that is present in, for example, healthy tissue, in contrast to a level of protein and/or mRNA present in the sample with unknown characteristics. In some embodiments, the reference sample comes from the same subject, but is from a different part of the subject than that being tested. In some embodiments, the reference sample is from a tissue area surrounding or adjacent to the cancer. In some embodiments, the reference sample is not from the subject being tested, but is a sample from a subject known to have, or not to have, a disorder in question (for example, a particular cancer). In some embodiments, the reference sample is from the same subject, but from a point in time before the subject developed cancer. In some embodiments, the reference sample is from a benign cancer sample, from the same or a different subject. When a negative reference sample is used for comparison, the level of expression or amount of the molecule in question in the negative reference sample will indicate a level at which one of skill in the art will appreciate, given the present disclosure, that there is no and/or a low level of the molecule. When a positive reference sample is used for comparison, the level of expression or amount of the molecule in question in the positive reference sample will indicate a level at which one of skill in the art will appreciate, given the present disclosure, that there is a level of the molecule.

The terms “benefit”, “clinical benefit”, “responsiveness”, and “therapeutic responsiveness” as used herein in the context of benefiting from or responding to administration of a therapeutic agent, can be measured by assessing various endpoints, e.g, inhibition, to some extent, of disease progression, including slowing down and complete arrest; reduction in the number of disease episodes and/or symptoms; reduction in lesion size; inhibition (that is, reduction, slowing down or complete stopping) of disease cell infiltration into adjacent peripheral organs and/or tissues, inhibition (that is, reduction, slowing down or complete stopping) of disease spread; relief, to some extent, of one or more symptoms associated with the disorder, increase in the length of disease-free presentation following treatment, for example, progression-free survival, increased overall survival; higher response rate, and/or decreased mortality at a given point of time following treatment. A subject or cancer that is “non-responsive” or “fails to respond” is one that has failed to meet the above noted qualifications to be “responsive”.

The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides comprised in the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification. In some embodiments, a polypeptide is a “complex” of a first polypeptide and a second polypeptide.

“NKp46” as used herein refers to any native, mature NKp46 that results from processing of a NKp46 precursor in a cell. The term includes NKp46 from any vertebrate source, including mammals such as primates (e.g., humans and cynomolgus or rhesus monkeys) and rodents (e.g., mice and rats), unless otherwise indicated. The term also includes naturally-occurring variants of NKp46, such as splice variants or allelic variants. A nonlimiting exemplary human NKp46 amino acid sequence is shown, e.g., in UniProt Accession No. O76036. See SEQ ID NO: 29. A nonlimiting exemplary mature human NKp46 sequence would be amino acids 22-304 of SEQ ID NO: 29. In some embodiments, NKp46 is expressed on NK cells, but is not expressed on CD4+ or CD8+ T-cells, B-cells, monocytes, or granulocytes NKp46 may serve to activate NK cells and trigger effector function.

The term “natural killer cell-mediated cytotoxicity” or “NK-mediated cytotoxicity” refers to killing of target cells by release of cytotoxic molecules from NK cells. These cytotoxic molecules, such as granzymes and perforin, may be stored in secretory lysosomes (also known as lytic granules) that are exocytosed from the NK cell when it interacts with a target cell. Exemplary endogenous targets of NK cells are virally infected or tumorigenic cells. Exocytosis of secretory lysosomes by NK cells is regulated to avoid indiscriminate cytotoxicity.

As used herein, “redirected NK-mediated cytotoxicity” refers to cytotoxicity of target cells by NK cells, wherein the target cell is not endogenously targeted by NK cells. In other words, redirected NK-mediated cytotoxicity refers to NK cytotoxicity that is directed towards cells that are not normally targets of NK cells. Redirected NK-mediated cytotoxicity can also refer to a greater cytotoxicity by a NK cell to a target cell compared to an endogenous NK response to this target cell. Redirected NK-mediated cytotoxicity can be mediated by an agent capable of redirecting NK cells towards a target cell, such as a polypeptide comprising at least one VHH domain that binds NKp46 and at least one VHH that binds an antigen on the target cell. As such, a polypeptide comprising at least one VHH domain that binds NKp46 and at least one VHH that binds an antigen on a target cell can be used to direct a NK cell to a target cell and stimulate NK-mediated cytotoxicity against the target cell.

As used herein “IL-2” or “Interleukin-2” refers to any native, mature IL-2 that results from processing of an IL-2 precursor in a cell. The term includes IL-2 from any vertebrate source, including mammals such as primates (e.g., humans and cynomolgus or rhesus monkeys) and rodents (e.g, mice and rats), unless otherwise indicated. The term also includes naturally-occurring variants of IL-2, such as splice variants or allelic variants. A nonlimiting exemplary human IL-2 amino acid sequence is shown, e.g., in GenBank Accession No NP_000577.2. See SEQ ID NO: 30 (mature form).

“Modified IL-2” as used herein refers to a polypeptide that differs from a wild type IL-2 amino acid sequence by a substitution at at least one amino acid position.

The term “IL-2 activity” or “biological activity” of IL-2, as used herein, includes any biological effect or at least one of the biologically relevant functions of IL-2 In some embodiments, IL-2 activity includes the ability of IL-2 to induce T cell proliferation and/or activate natural killer (NK) cells. Nonlimiting exemplary IL-2 activities include increasing pSTAT5 expression, increasing proliferation of lymphocytes and other peripheral blood mononuclear cells (PBMC's), increasing Granzyme B expression by NK cells.

As used herein, a “NKp46-binding polypeptide” and a “NKp46-targeted polypeptide” are used interchangeably to refer to polypeptides comprising a binding domain that binds, such as specifically binds, to NKp46.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. A single-domain antibody (sdAb) or VHH-containing polypeptide “specifically binds” or “preferentially binds” to a target if it binds with greater affinity, avidity, more readily, and/or with greater duration than it binds to other substances. For example, a sdAb or VHH-containing polypeptide that specifically or preferentially binds to a NKp46 epitope is a sdAb or VHH-containing polypeptide that binds this epitope with greater affinity, avidity, more readily, and/or with greater duration than it binds to other NKp46 epitopes or non-NKp46 epitopes. It is also understood by reading this definition that; for example, a sdAb or VHH-containing polypeptide that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 10% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control over the same period of time.

As used herein, the term “epitope” refers to a site on a target molecule (for example, an antigen, such as a protein, nucleic acid, carbohydrate or lipid) to which an antigen-binding molecule (for example, a sdAb or VHH-containing polypeptide) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some embodiments, an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between a residue of the antigen-binding molecule and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antigen-binding molecule. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

A “nonlinear epitope” or “conformational epitope” comprises noncontiguous polypeptides, amino acids and/or sugars within the antigenic protein to which an antigen-binding molecule specific to the epitope binds. In some embodiments, at least one of the residues will be noncontiguous with the other noted residues of the epitope; however, one or more of the residues can also be contiguous with the other residues.

A “linear epitope” comprises contiguous polypeptides, amino acids and/or sugars within the antigenic protein to which an antigen-binding molecule specific to the epitope binds. It is noted that, in some embodiments, not every one of the residues within the linear epitope need be directly bound (or involved in a bond) by the antigen-binding molecule. In some embodiments, linear epitopes can be from immunizations with a peptide that effectively consisted of the sequence of the linear epitope, or from structural sections of a protein that are relatively isolated from the remainder of the protein (such that the antigen-binding molecule can interact, at least primarily), just with that sequence section.

The term “antibody” is used in the broadest sense and encompass various polypeptides that comprise antibody-like antigen-binding domains, including but not limited to conventional antibodies (typically comprising at least one heavy chain and at least one light chain), single-domain antibodies (sdAbs, comprising at least one VHH domain and an Fc region), VHH-containing polypeptides (polypeptides comprising at least one VHH domain), and fragments of any of the foregoing so long as they exhibit the desired antigen-binding activity. In some embodiments, an antibody comprises a dimerization domain. Such dimerization domains include, but are not limited to, heavy chain constant domains (comprising C_{H}1, hinge, C_{H}2, and C_{H}3, where C_{H}1 typically pairs with a light chain constant domain, CL, while the C_{H}3 and/or hinge mediates dimerization) and Fc regions (comprising hinge, C_{H}2, and C_{H}3, where the C_{H}3 and/or hinge mediates dimerization).

The term antibody also includes, but is not limited to, chimeric antibodies, humanized antibodies, and antibodies of various species such as camelid (including llama), shark, mouse, human, cynomolgus monkey, etc.

The term “antigen-binding domain” as used herein refers to a portion of an antibody sufficient to bind antigen. In some embodiments, an antigen binding domain of a conventional antibody comprises three heavy chain CDRs and three light chain CDRs. Thus, in some embodiments, an antigen binding domain comprises a heavy chain variable region comprising CDR1-FR2-CDR2-FR3-CDR3, and any portions of FR1 and/or FR4 required to maintain binding to antigen, and a light chain variable region comprising CDR1-FR2-CDR2-FR3-CDR3, and any portions of FR1 and/or FR4 required to maintain binding to antigen. In some embodiments, an antigen-binding domain of an sdAb or VHH-containing polypeptide comprises three CDRs of a VHH domain. Thus, in some embodiments, an antigen binding domain of an sdAb or VHH-containing polypeptide comprises a VHH domain comprising (CDR1-FR2-CDR2-FR3-CDR3, and any portions of FR1 and/or FR4 required to maintain binding to antigen.

The term “VHH” or “VHH domain” or “VHH antigen-binding domain” as used herein refers to the antigen-binding portion of a single-domain antibody, such as a camelid antibody or shark antibody. In some embodiments, a VHH comprises three CDRs and four framework regions, designated FR1, CDR1, FR2, CDR2, FR3, CDR3, and FR4. In some embodiments, a VHH may be truncated at the N-terminus or C-terminus such that it comprises only a partial FR1 and/or FR4, or lacks one or both of those framework regions, so long as the VHH substantially maintains antigen binding and specificity.

The terms “single domain antibody” and “sdAb” are used interchangeably herein to refer to an antibody comprising at least one monomeric domain, such as a VHH domain, without a light chain, and an Fc region. In some embodiments, an sdAb is a dimer of two polypeptides wherein each polypeptide comprises at least one VHH domain and an Fc region. As used herein, the terms “single domain antibody” and “sdAb” encompass polypeptides that comprise multiple VHH domains, such as a polypeptide having the structure VHH₁-VHH₂-Fc or VHH₁-VHH₂-VHH₃-Fc, wherein VHH₁, VHH₂, and VHH₃ may be the same or different.

The term “VHH-containing polypeptide” refers to a polypeptide that comprises at least one VHH domain. In some embodiments, a VHH polypeptide comprises two, three, or four or more VHH domains, wherein each VHH domain may be the same or different. In some embodiments, a VHH-containing polypeptide comprises an Fc region. In some such embodiments, the VHH-containing polypeptide may be referred to as an sdAb. Further, in some such embodiments, the VHH polypeptide may form a dimer. Nonlimiting structures of VHH-containing polypeptides, which are also sdAbs, include VHH₁-Fe, VHH₁-VHH₂-Fc, and VHH₁-VHH₂-VHH₃-Fc, wherein VHH₁, VHH₂, and VHH₃ may be the same or different. In some embodiments of such structures, one VHH may be connected to another VHH by a linker, or one VHH may be connected to the Fc by a linker. In some such embodiments, the linker comprises 1-20 amino acids, preferably 1-20 amino acids predominantly composed of glycine and, optionally, serine. In some embodiments, the linker comprises: Gly-Gly-Gly-Gly (SEQ ID NO: 45), Gly-Gly-Ser-Gly-Gly-Ser (SEQ ID NO: 46), and/or Gly-Gly-Ser-Ser-Gly-Ser (SEQ ID NO: 47). In some embodiments, when a VHH-containing polypeptide comprises an Fc, it forms a dimer. Thus, the structure VHH₁-VHH₂-Fc, if it forms a dimer, is considered to be tetavalent (i.e., the dimer has four VHH domains). Similarly, the structure VHH₁-VHH₂-VHH₃-Fc, if it forms a dimer, is considered to be hexavalent (i.e., the dimer has six VHH domains).

The term “monoclonal antibody” refers to an antibody (including an sdAb or VHH-containing polypeptide) of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, and/or the contact definition. A VHH comprises three CDRs, designated CDR1, CDR2, and CDR3 in some embodiments, the CDRs are defined in accordance with the AbM definition.

The term “heavy chain constant region” as used herein refers to a region comprising at least three heavy chain constant domains, C_{H}1, hinge, C_{H}2, and C_{H}3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies, and IgM antibodies include, but are not limited to, IgM1 and IgM2.

A “Fc region” as used herein refers to a portion of a heavy chain constant region comprising C_{H}2 and C_{H}3 In some embodiments, an Fc region comprises a hinge, (C_{H}2, and C_{H}3. In some embodiments, an Fc region does not comprise a hinge. In various embodiments, when an Fc region comprises a hinge, the hinge and/or C_{H}3 mediates dimerization between two Fc-containing polypeptides. In various embodiments, when an Fc region does not comprise a hinge, the C_{H}3 mediates dimerization between two Fc-containing polypeptides. An Fc region may be of any antibody heavy chain constant region isotype discussed herein. In some embodiments, an Fc region is an IgG1, IgG2, IgG3, or IgG4.

An “acceptor human framework” as used herein is a framework comprising the amino acid sequence of a heavy chain variable domain (V_{H}) framework derived from a human immunoglobulin framework or a human consensus framework, as discussed herein. An acceptor human framework derived from a human immunoglobulin framework or a human consensus framework can comprise the same amino acid sequence thereof, or it can contain amino acid sequence changes. In some embodiments, the number of amino acid changes are fewer than 10, or fewer than 9, or fewer than 8, or fewer than 7, or fewer than 6, or fewer than 5, or fewer than 4, or fewer than 3, across all of the human frameworks in a single antigen binding domain, such as a VHH.

“Affinity” refers to the strength of the sum total of noncovalent interactions between a single binding site of a molecule (for example, an antibody, such as an sdAb, or VHH-containing polypeptide) and its binding partner (for example, an antigen). The affinity or the apparent affinity of a molecule X for its partner Y can generally be represented by the dissociation constant (K_{d}) or the K_{d-apparent}, respectively. Affinity can be measured by common methods known in the art (such as, for example, ELISA K_{d}, KinExA, flow cytometry, and/or surface plasmon resonance devices), including those described herein. Such methods include, but are not limited to, methods involving BIAcore®, Octet®, or flow cytometry.

The term “K_{d}”, as used herein, refers to the equilibrium dissociation constant of an antigen-binding molecule/antigen interaction. When the term “K_{d}” is used herein, it includes K_{d} and K_{d-apparent}.

In some embodiments, the K_{d} of the antigen-binding molecule is measured by flow cytometry using an antigen-expressing cell line and fitting the mean fluorescence measured at each antibody concentration to a non-linear one-site binding equation (Prism Software GraphPad) In some such embodiments, the K_{d} is K_{d-apparent}.

The term “biological activity” refers to any one or more biological properties of a molecule (whether present naturally as found in vivo, or provided or enabled by recombinant means). Biological properties include, but are not limited to, binding a ligand, inducing or increasing cell proliferation (such as NK cell proliferation), inducing or increasing cell activation (such as NK cell activation), and inducing or increasing expression of cytokines.

An “agonist” or “activating” antibody is one that increases and/or activates a biological activity of the target antigen. In some embodiments, the agonist antibody binds to an antigen and increases its biologically activity by at least about 20%, 40%, 60%, 80%, 85% or more.

An “antagonist”, a “blocking” or “neutralizing” antibody is one that inhibits, decreases and/or inactivates a biological activity of the target antigen. In some embodiments, the neutralizing antibody binds to an antigen and reduces its biologically activity by at least about 20%, 40%, 60%, 80%, 85% 90%, 95%, 99% or more.

An “affinity matured” sdAb or VHH-containing polypeptide refers to a sdAb or VHH-containing polypeptide with one or more alterations in one or more CDRs compared to a parent sdAb or VHH-containing polypeptide that does not possess such alterations, such alterations resulting in an improvement in the affinity of the sdAb or VHH-containing polypeptide for antigen.

A “humanized VHH” as used herein refers to a VHH in which one or more framework regions have been substantially replaced with human framework regions. In some instances, certain framework region (FR) residues of the human immunoglobulin are replaced by corresponding non-human residues. Furthermore, the humanized VHH can comprise residues that are found neither in the original VHH nor in the human framework sequences, but are included to further refine and optimize sdAb VHH-containing polypeptide performance. In some embodiments, a humanized sdAb or VHH-containing polypeptide comprises a human Fc region. As will be appreciated, a humanized sequence can be identified by its primary sequence and does not necessarily denote the process by which the antibody was created.

An “effector-positive Fc region” possesses an “effector function” of a native sequence Fc region. Exemplary “effector functions” include Fc receptor binding; Clq binding and complement dependent cytotoxicity (CDC); Fc receptor binding; antibody-dependent cell-mediated cytotoxicity (ADCC); phagocytosis; down regulation of cell surface receptors (for example B-cell receptor), and B-cell activation, etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region, native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fe region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (See, for example, Daeron, Annu. Rev. Immunol. 15:203-234 (1997)). FcRs are reviewed, for example, in Ravetch and Kinet, Annu. Rev. Immunol 9.457-92 (1991); Capel et al., Immunomethods 4:25-34 (1994); and de Haas el al., J. Lab. Clin. Med. 126:330-41 (1995). Other FcRs, including those to be identified in the future, are encompassed by the term “FcR” herein. For example, the term “Fc receptor” or “FcR” also includes the neonatal receptor, FcRn, which is responsible for the transfer of maternal IgGs to the fetus (Guyer et al, J. Immunol. 117:587 (1976) and Kim et al., J. Immunol. 24:249 (1994)) and regulation of homeostasis of immunoglobulins. Methods of measuring binding to FcRn are known (see, for example, Ghetie and Ward, Immunol. Today 18(12):592-598 (1997), Ghetie et al, Nature Biotechnology, 15(7):637-640 (1997); Hinton el al., J. Biol. Chem. 279(8):6213-6216 (2004); WO 2004/92219 (Hinton et al.)

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved antigen binding, decreased immunogenicity, or improved ADCC or CDC

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys, Arg |
| Asp (D) | Glu; Asn |
| Cys (C) | Ser; Ala |
| Gln (Q) | Asn; Glu |
| Glu (E) | Asp, Gln |
| Gly (G) | Ala |
| His (H) | Asn; Gln; Lys; Arg |
| Ile (I) | Leu; Val, Met; Ala; Phe; Norleucine |
| Leu (L) | Norleucine; Ile; Val; Met; Ala; Phe |
| Lys (K) | Arg, Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic. Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E, CHO-DG44, CHO-K1, CHO-S, and CHO-DS cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) a provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” and “subject” are used interchangeably herein to refer to an animal; for example, a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

A “disease” or “disorder” as used herein refers to a condition where treatment is needed and/or desired.

The term “tumor cell”, “cancer cell”, “cancer”, “tumor”, and/or “neoplasm”, unless otherwise designated, are used herein interchangeably and refer to a cell (or cells) exhibiting an uncontrolled growth and/or abnormal increased cell survival and/or inhibition of apoptosis which interferes with the normal functioning of bodily organs and systems. Included in this definition are benign and malignant cancers, hematologic cancers such as leukemias, lymphomas, and multiple myelomas, polyps, hyperplasia, as well as dormant tumors or micrometastases.

The terms “cancer” and “tumor” encompass solid and hematological/lymphatic cancers and also encompass malignant, pre-malignant, and benign growth, such as dysplasia. Exemplary cancers include, but are not limited to: basal cell carcinoma, biliary tract cancer; bladder cancer; bone cancer; brain and central nervous system cancer; breast cancer; cancer of the peritoneum, cervical cancer, choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastomna; hepatic carcinoma; hepatoma; intra-epithelial neoplasm; kidney or renal cancer; larynx cancer; leukemia; liver cancer, lung cancer (e.g., small-cell lung cancer, non-small cell lung cancer, adenocarcinoma of the lung, and squamous carcinoma of the lung), melanoma; myeloma; neuroblastoma; oral cavity cancer (lip, tongue, mouth, and pharynx); ovarian cancer; pancreatic cancer; prostate cancer, retinoblastoma; rhabdomyosarcoma; rectal cancer; cancer of the respiratory system; salivary gland carcinoma; sarcoma; skin cancer; squamous cell cancer; stomach cancer, testicular cancer; thyroid cancer; uterine or endometrial cancer, cancer of the urinary system; vulval cancer; lymphoma including Hodgkin's and non-Hodgkin's lymphoma, as well as B-cell lymphoma (including low grade/follicular non-Hodgkin's lymphoma (NHL), small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia, acute myeloid leukemia (AML); chronic lymphocytic leukemia (CLL); acute lymphoblastic leukemia (ALL); Hairy cell leukemia; chronic myeloblastic leukemia; as well as other carcinomas and sarcomas, and post-transplant lymphoproliferative disorder (PTLD), as well as abnormal vascular proliferation associated with phakomatoses, edema (such as that associated with brain tumors), and Meigs' syndrome.

The term “non-tumor cell” as used herein refers to a normal cells or tissue. Exemplary non-tumor cells include, but are not limited to: T-cells, B-cells, natural killer (NK) cells, natural killer T (NKT) cells, dendritic cells, monocytes, macrophages, epithelial cells, fibroblasts, hepatocytes, interstitial kidney cells, fibroblast-like synoviocytes, osteoblasts, and cells located in the breast, skeletal muscle, pancreas, stomach, ovary, small intestines, placenta, uterus, testis, kidney, lung, heart, brain, liver, prostate, colon, lymphoid organs, bone, and bone-derived mesenchymal stem cells. The term “a cell or tissue located in the periphery” as used herein refers to non-tumor cells not located near tumor cells and/or within the tumor microenvironment.

The term “cells or tissue within the tumor microenvironment” as used herein refers to the cells, molecules, extracellular matrix and/or blood vessels that surround and/or feed a tumor cell. Exemplary cells or tissue within the tumor microenvironment include, but are not limited to: tumor vasculature; tumor-infiltrating lymphocytes; fibroblast reticular cells; endothelial progenitor cells (EPC), cancer-associated fibroblasts; pericytes; other stromal cells; components of the extracellular matrix (ECM); dendritic cells, antigen presenting cells; T-cells; regulatory T-cells (Treg cells); NK cells; macrophages, neutrophils; myeloid-derived suppressor cells (MDSCs) and other immune cells located proximal to a tumor. Methods for identifying tumor cells, and/or cells/tissues located within the tumor microenvironment are well known in the art, as described herein, below.

The term “infectious disease,” as used herein refers to a disease caused by a pathogenic virus, bacteria, or fungus.

In some embodiments, an “increase” or “decrease” refers to a statistically significant increase or decrease, respectively. As will be clear to the skilled person, “modulating” can also involve effecting a change (which can either be an increase or a decrease) in affinity, avidity, specificity and/or selectivity of a target or antigen, for one or more of its ligands, binding partners, partners for association into a homomultimeric or heteromultimeric form, or substrates; effecting a change (which can either be an increase or a decrease) in the sensitivity of the target or antigen for one or more conditions in the medium or surroundings in which the target or antigen is present (such as pH, ion strength, the presence of co-factors, etc.); and/or cellular proliferation or cytokine production, compared to the same conditions but without the presence of a test agent. This can be determined in any suitable manner and/or using any suitable assay known per se or described herein, depending on the target involved.

As used herein, “an immune response” is meant to encompass cellular and/or humoral immune responses that are sufficient to inhibit or prevent onset or ameliorate the symptoms of disease (for example, an infectious disease or cancer or cancer metastasis). “An immune response” can encompass aspects of both the innate and adaptive immune systems.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering a therapeutic agent. “Ameliorating” also includes shortening or reduction in duration of a symptom.

The term “anti-cancer agent” is used herein in its broadest sense to refer to agents that are used in the treatment of one or more cancers. Exemplary classes of such agents in include, but are not limited to, chemotherapeutic agents, anti-cancer biologics (such as cytokines, receptor extracellular domain-Fe fusions, and antibodies), radiation therapy, CAR-T therapy, therapeutic oligonucleotides (such as antisense oligonucleotides and siRNAs) and oncolytic viruses.

The term “biological sample” means a quantity of a substance from a living thing or formerly living thing. Such substances include, but are not limited to, blood, (for example, whole blood), plasma, serum, urine, amniotic fluid, synovial fluid, endothelial cells, leukocytes, monocytes, other cells, organs, tissues, bone marrow, lymph nodes and spleen.

The term “control” or “reference” refers to a composition known to not contain an analyte (“negative control”) or to contain an analyte (“positive control”). A positive control can comprise a known concentration of analyte.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce”, “inhibit”, or “prevent” do not denote or require complete prevention over all time, but just over the time period being measured.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result.

The terms “pharmaceutical formulation” and “pharmaceutical composition” are used interchangeably and refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and are compatible with other ingredients of the formulation. The pharmaceutically acceptable carrier is appropriate for the formulation employed.

Administration “in combination with” one or more further therapeutic agents includes simultaneous (concurrent) and sequential administration in any order.

The term “concurrently” is used herein to refer to administration of two or more therapeutic agents, where at least part of the administration overlaps in time, or where the administration of one therapeutic agent falls within a short period of time relative to administration of the other therapeutic agent, or wherein the therapeutic effects of both agents overlap for at least a period of time.

The term “sequentially” is used herein to refer to administration of two or more therapeutic agents that does not overlap in time, or wherein the therapeutic effects of the agents do not overlap.

As used herein, “in conjunction with” refers to administration of one treatment modality in addition to another treatment modality. As such, “in conjunction with” refers to administration of one treatment modality before, during, or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (for example, a package or container) or kit comprising at least one reagent, for example, a medicament for treatment of a disease or disorder (for example, cancer), or a probe for specifically detecting a biomarker described herein. In some embodiments, the manufacture or kit is promoted, distributed, or sold as a unit for performing the methods described herein.

The terms “label” and “detectable label” mean a moiety attached, for example, to an antibody or antigen to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

### Exemplary NKp46-Targeted Modified IL-2 Polypeptides

NKp46-targeted modified IL-2 polypeptides are provided herein. In various embodiments, the NKp46-targeted modified IL-2 polypeptides comprise at least one VHH domain that binds NKp46 and comprise a modified IL-2. In some embodiments, the NKp46 is human NKp46. In some embodiments, a NKp46-targeted modified IL-2 polypeptide provided herein comprises one, two, three, four, five, six, seven, or eight VHH domains that bind NKp46. In some embodiments, a NKp46-targeted modified IL-2 polypeptide provided herein comprises one, two, three, or four VHH domains that bind NKp46. NKp46-targeted modified IL-2 polypeptides may comprise one or more VHH domains that bind one or more target proteins other than NKp46 Such polypeptides may be referred to as “multispecific” polypeptides.

In some embodiments, a NKp46-targeted modified IL-2 polypeptide comprises at least one VHH domain that binds NKp46, an Fc region, and a modified IL-2. In some embodiments, a NKp46-targeted modified IL-2 polypeptide provided herein comprises one, two, three, or four VHH domains, an Fc region, and a modified IL-2 In some embodiments, an Fc region mediates dimerization of the NKp46-targeted modified IL-2 polypeptide at physiological conditions such that a dimer is formed that doubles the number of NKp46 binding sites. For example, a NKp46-targeted modified IL-2 polypeptide comprising three VHH domains that bind NKp46, an Fc region, and a modified IL-2 is trivalent as a monomer, but at physiological conditions, the Fc region may mediate dimerization, such that the NKp46-targeted modified IL-2 polypeptide exists as a hexavalent dimer under such conditions.

In some embodiments, a NKp46-targeted modified IL-2 polypeptide comprises at least two VHH domains, wherein a first VHH domain binds a first epitope of NKp46 and a second VHH domain binds a second epitope of NKp46. When the NKp46-targeted modified IL-2 polypeptide comprises a VHH domain that binds a first epitope of NKp46 and a VHH domain that binds a second epitope of NKp46, the NKp46-targeted modified IL-2 polypeptide may be referred to as “biepitopic” or “bispecific.” In some embodiments, a NKp46-targeted modified IL-2 polypeptide comprises at least two VHH domains, wherein a first VHH domain binds NKp46 and a second VHH domain binds an antigen other than NKp46. Such polypeptides may be referred to as “bispecific” or “multispecific.”

In some embodiments, a NKp46-targeted modified IL-2 polypeptide is a complex of a first polypeptide comprising a first VHH domain that binds NKp46, a first Fc domain, and a modified IL-2 polypeptide; and a second polypeptide comprising a second VHH domain that binds NKp46 and a second Fc domain. In some such embodiments, the first or second Fc domain comprises “knob” mutation(s) and the other Fc domain comprises “hole” mutation(s). Thus, in some embodiments, a NKp46-targeted modified IL-2 polypeptide is a complex of a first polypeptide and a second polypeptide, wherein the complex comprises two NKp46-binding VHH domains and one modified IL-2 polypeptide. In some embodiments, a NKp46-targeted modified IL-2 polypeptide is a complex of a first polypeptide comprising a first VHH domain that binds NKp46 and a first Fc domain; and a second polypeptide comprising a second Fc domain, a modified IL-2 polypeptide, and optionally, a second VHH domain. In some such embodiments, the first or second Fc domain comprises “knob” mutation(s) and the other Fc domain comprises “hole” mutation(s). Thus, in some embodiments, a NKp46-targeted modified IL-2 complex comprises a NKp46-binding VHH domain and one modified IL-2 polypeptide within different polypeptides of the complex.

Exemplary NKp46-Targeted Domains that Bind NKp46

In various embodiments, a VHH domain that binds NKp46 comprises a CDR1 sequence of SEQ ID NO: 17; a CDR2 sequence selected from SEQ ID NOs: 18, 19, 20, and 21; and a CDR3 sequence selected from SEQ ID NOs: 22, 23, 24, 25, 26, and 27. In various embodiments, a VHH domain that binds NKp46 comprises CDR1, CDR2, and CDR3 sequences selected from: SEQ ID NOs: 17, 18, and 22; SEQ ID NOs: 17, 19, and 22, SEQ ID) NOs: 17, 20, and 22; SEQ ID NOs: 17, 21, and 22; SEQ ID NOs: 17, 18, and 23; SEQ ID NOs: 17, 18, and 24; SEQ ID NOs: 17, 18, and 25; SEQ ID NOs: 17, 18, and 26, and SEQ ID NOs: 17, 18, and 27.

In some embodiments, a VHH domain that binds NKp46 comprises an amino acid sequence that is at least 85%, at least 90%, at least 95%, at least 96%, at least 97%, at least 98%, at least 99% identical to an amino acid sequence selected from SEQ ID NOs: 1, 2, 3, 4, 5, 6, 7, 8, 9, 10, 11, 12, 13, 14, 15, and 16 In some embodiments, a VHH domain that binds NKp46 comprises an amino acid sequence selected from SEQ ID NOs: 1, 2, 3, 4, 5, 6, 7, 8, 9, 10, 11, 12, 13, 14, 15, and 16. In some embodiments, a VHH domain that binds NKp46 comprises an amino acid sequence selected from SEQ ID NOs: 1, 2, 3, 4, 5, 6, 7, 8, 9, 10, 11, 12, 13, 14, 15, and 16, wherein the VHH domain comprises a K125D, K125E, or K125R mutation.

Nonlimiting exemplary NKp46-targeted modified IL-2 polypeptides are shown in Table 2. The sequences for the indicated single-domain antibodies are shown in the Table of Certain Sequences herein.

TABLE 2
Polypeptides comprising at least one VHH that binds NKp46

| Name | CDRs | VHH |
|---|---|---|
| 5D7 | SEQ ID NOs: 17, 18, and 22 | SEQ ID NO: 1 |
| hz5D7v1 | SEQ ID NOs: 17, 18, and 22 | SEQ ID NO: 2 |
| hz5D7v2 | SEQ ID NOs: 17, 18, and 22 | SEQ ID NO: 3 |
| hz5D7v3 | SEQ ID NOs: 17, 18, and 22 | SEQ ID NO: 4 |
| hz5D7v4 | SEQ ID NOs: 17, 18, and 22 | SEQ ID NO: 5 |
| hz5D7v7 | SEQ ID NOs: 17, 19, and 22 | SEQ ID NO: 6 |
| hz5D7v8 | SEQ ID NOs: 17, 20, and 22 | SEQ ID NO: 7 |
| hz5D7v9 | SEQ ID NOs: 17, 21, and 22 | SEQ ID NO: 8 |
| hz5D7v10 | SEQ ID NOs: 17, 18, and 22 | SEQ ID NO: 9 |
| hz5D7v11 | SEQ ID NOs: 17, 18, and 23 | SEQ ID NO: 10 |
| hz5D7v12 | SEQ ID NOs: 17, 18, and 24 | SEQ ID NO: 11 |
| hz5D7v13 | SEQ ID NOs: 17, 18, and 25 | SEQ ID NO: 12 |
| hz5D7v14 | SEQ ID NOs: 17, 18, and 26 | SEQ ID NO: 13 |
| hz5D7v15 | SEQ ID NOs: 17, 18, and 27 | SEQ ID NO: 14 |
| hz5D7v17 | SEQ ID NOs: 17, 18, and 24 | SBQ ID NO: 15 |
| hz5D7v12L | SEQ ID NOs: 17, 18, and 24 | SEQ ID NO: 16 |

In various embodiments, a NKp46-targeted modified IL-2 polypeptide comprises one, two, three, or four VHH domains that bind NKp46.

In various embodiments, a NKp46-targeted modified IL-2 polypeptide comprises at least one VHH domain that binds NKp46 and at least one binding domain that binds an antigen other than NKp46. In some embodiments, the at least one binding-domain that binds an antigen other than NKp46 is a VHH domain. For example, in some embodiments, the at least one VHH domain that binds an antigen other than NKp46 binds a T-cell antigen, a natural killer cell antigen that is not NKp46, or a tumor antigen.

In some such embodiments, the NKp46-targeted modified IL-2 polypeptide may be referred to as a multispecific antibody. By multispecific, it is meant that a NKp46-targeted modified IL-2 polypeptide can bind to one or more other antigen, in addition to NKp46.

In some embodiments, a NKp46-targeted modified IL-2 polypeptide may mediate more than one biological function, wherein one biological functional is binding to NKp46 and one biological function is IL-2 activity. In some embodiments, a NKp46-targeted modified IL-2 polypeptide is trifunctional (having three functions). For example, as described below, a NKp46-targeted modified IL-2 polypeptide may bind another antigen (such as a tumor antigen) and also have IL-2 activity.

In some embodiments, the NKp46-targeted modified IL-2 polypeptide comprises at least one binding domain that binds a cancer cell. In some embodiments, the NKp46-targeted modified IL-2 polypeptide comprises at least one binding domain that binds a tumor antigen in some embodiments, the NKp46-targeted modified IL-2 polypeptide comprises at least one binding domain that binds to 1-92-LFA-3, 5T4, Alpha-4 integrin, Alpha-V integrin, alpha4beta1 integrin, alpha4beta7 integrin, AGR2, Anti-Lewis-Y, Apelin J receptor, APRIL, B7-13, B7-1I4, B7-H6, BAFF, BCMA, BTLA, C5 complement, C-242, CA9, CA19-9, (Lewis a), Carbonic anhydrase 9, CD2, CD3, CD6, CD9, CD11a, CD19, CD20, CD22, CD24, CD25, CD27, CD28, CD30, CD33, CD38, CD39, CD40, CD40L, CD41, CD44, CD44v6, CD47, CD51, CD52, CD56, CD64, CD70, CD71, CD73, CD74, CD80, CD81, CD86, CD95, CD117, CD123, CD125, CD132, (IL-2RG), CD133, CD137, CD138, CD166, CD172A, CD248, CDH6, CEACAM5 (CEA), CEACAM6 (NCA-90), CLAUDIN-3, CLAUDIN-4, cMet, Collagen, Cripto, CSFR, CSFR-1, CTLA-4, CTGF, CXCL10, CXCL13, CXCR1, CXCR2, CXCR4, CYR61, DL44, DLK1, DLL3, DLL4, DPP-4, DSG1, EDA, EDB, EGFR, EGFRviii, Endothelin B receptor (ETBR), ENPP3, EpCAM, EPHA2, EPHB2, ERBB3, F protein of RSV, FAP, FAS, FcRH5, FGF-2, FGF8, FGFR1, FGFR2, FGFR3, FGFR4, FLT-3, Folate receptor alpha (FRα), GAL3ST1, G-CSF, G-CSFR, GD2, GITR, GLUT1, GLUT4, GM-CSF, GM-CSFR, GP IIb/IIIa receptors, Gp130, GPIIB/IIIA, GPNMB, GPRC5D, GRP78, HAVCAR1, HER2/neu, HER3, HER4, HGF, hGH, HVEM, Hyaluronidase, ICOS, IFNalpha, IFNbeta, IFNgamma, IgE, IgE Receptor (FceRI), IGF, IGF1R, IL1B, IL1R, IL2, IL11, IL12, IL12p40, IL-12R, IL-12Rbeta1, IL13, IL13R, IL15, IL17, IL18, IL21, IL23, IL23R, IL27/L27R (wsx1), IL29, IL-31R, IL31/IL31R, IL2R, 114, IL4R, IL6, IL6R, Insulin Receptor, Jagged Ligands, Jagged 1, Jagged 2, KISS1-R, LAG-3, LIF-R, Lewis X, LIGHT, LRP4, LRRC26, Ly6G6D, LyPD1, MCSP, Mesothelin, MICA, MICB, MRP4, MUC1, Mucin-16 (MUC16, CA-125), Na/K ATPase, NGF, Nicastrin, NKG2A, Notch Receptors, Notch 1, Notch 2, Notch 3, Notch 4, NOV, OSM-R, OX-40, PAR2, PDGF-AA, PDGF-BB, PDGFRalpha, PDGFRbeta, PD-1, PD-L1, PD-L2, Phosphatidyl-serine, P1GF, PSCA, PSMA, PSGR, RAAG12, RAGE, SLC44A4, Sphingosine 1 Phosphate, STEAP1, STEAP2, TAG-72, TAPA1, TEM-8, TGFbeta, TGFbeta receptor 1 (TGFBR1), TGFbeta receptor 2 (TGFBR2), TIGIT, TIM-3, TLR2, TLR4, TLR6, TLR7, TLR8, TLR9, TMEM31, TNFalpha, TNFR, TNFRS12A, TRAIL-R1, TRAIL-R2, Transferrin, Transferrin receptor, TRK-A, TRK-B, TROP-2 uPAR, VAP1, VCAM-1, VEGF, VEGF-A, VEGF-B, VEGF-C, VEGF-D, VEGFR1, VEGFR2, VEGFR3, VISTA, WISP-1, WISP-2, or WISP-3. In some embodiments, the NKp46-targeted modified IL-2 polypeptide comprises at least one binding domain that binds a virally infected cell that is expressing viral proteins on the cell surface.

In some embodiments, a binding domain comprised in a NKp46-targeted modified IL-2 polypeptide, wherein the binding domain binds a cell that is not a NK cell, may be termed a “secondary targeting domain.” As such, a secondary targeting domain can target a NKp46-targeted modified IL-2 polypeptide to a cell of interest and redirect NK-mediated cytotoxicity towards this cell that expresses an antigen capable of binding the secondary targeting domain. For example, a secondary targeting domain of a NKp46-targeted modified IL-2 polypeptide may be an antibody against a tumor antigen, and binding of the NKp46 binding polypeptide to a cancer cell expressing the tumor antigen redirects NK-mediated cytotoxicity towards this cancer cell. In some embodiments, the secondary targeting domain is a VHH, single domain antibody, scFv, Fab, or any other type of antibody. In some embodiments, a secondary targeting domain is not an antibody. In some embodiments, the secondary targeting domain is a natural cognate binding partner, engineered extracellular binding, an Anticalin (engineered lipocalin), a Darpin, a Fynomer, a Centyrin (engineered fibroneticin III domain), a cystine-knot domain, an Affilin, an Affibody, or an engineered CH3 domain.

In some embodiments, a NKp46-targeted polypeptide comprises a binding domain, such as a VHH domain, that binds to TGFbeta receptor 1, TGFbeta receptor 2, or NKG2A.

In some embodiments, a NKp46-targeted polypeptide comprises at least one VHH domain described herein fused to an Fc region. In some embodiments, the Fe region has a sequence selected from SEQ ID NOs: 44, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 85, 86, 87, 88, and 89. In some such embodiments, the Fc region further comprises a C-terminal lysine. In some embodiments, the C-terminal amino acid of the Fc region is an amino acid other than lysine.

In some embodiments, a VHH domain that binds NKp46 is humanized. Humanized antibodies (such as sdAbs or VHH-containing polypeptides) are useful as therapeutic molecules because humanized antibodies reduce or eliminate the human immune response to non-human antibodies, which can result in an immune response to an antibody therapeutic, and decreased effectiveness of the therapeutic. Generally, a humanized antibody comprises one or more variable domains in which CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (for example, the antibody from which the CDR residues are derived), for example, to restore or improve antibody specificity or affinity.

Humanized antibodies and methods of making them are reviewed, for example, in Almagro and Fransson, (2008) Front. Biosci. 13: 1619-1633, and are further described, for example, in Riechmann et al, (1988) Nature 332:323-329: Queen et al., (1989) Proc. Natl Acad. Sci. USA 86: 10029-10033; U.S. Pat. Nos. 5,821,337, 7,527,791, 6,982,321, and 7,087,409; Kashmiri el al., (2005) Methods 36:25-34; Padlan, (1991) Mol. Immunol. 28.489-498 (describing “resurfacing”). Dall'Acqua et al., (2005) Method 36.43-60 (describing “FR shuffling”); and Osbourn e al., (2005) Methods 36:61-68 and Klimka et al., (2000) Br. J. Cancer, 83:252-260 (describing the “guided selection” approach to FR shuffling).

Human framework regions that can be used for humanization include but are not limited to: framework regions selected using the “best-fit” method (see, for example, Sims et al. (1993) J. Immunol. 151:2296); framework regions derived from the consensus sequence of human antibodies of a particular subgroup of heavy chain variable regions (see, for example, Carter et al. (1992) Proc. Natl. Acad. Sci. USA, 89:4285; and Presta et al. (1993) J. Immunol, 151:2623), human mature (somatically mutated) framework regions or human germline framework regions (see, for example, Almagro and Fransson, (2008) Front. Biosci. 13:1619-1633); and framework regions derived from screening FR libraries (see, for example, Baca el al., (1997) J. Biol. Chem. 272: 10678-10684 and Rosok et al, (1996) J. Biol. Chem. 271:22611-22618). Typically, the FR regions of a VHH are replaced with human FR regions to make a humanized VHH. In some embodiments, certain FR residues of the human FR are replaced in order to improve one or more properties of the humanized VHH. VHH domains with such replaced residues are still referred to herein as “humanized.”

### Exemplary Modified IL-2 Polypeptides

NKp46-targeted modified IL-2 polypeptides comprising a NKP46-binding VHH and a modified IL-2 are provided herein. In some such embodiments, the NKP46-targeted modified IL-2 polypeptide comprises a NKp46-binding VHH, an Fc region, and a modified IL-2. In some embodiments, the modified IL-2 comprises at least one amino acid substitution that reduces the affinity of the modified IL-2 for an IL-2 receptor compared to a wild type IL-2. In various embodiments, the polypeptide comprising a modified IL-2 provided herein is an agonist of an IL-2R. In some embodiments, the modified IL-2 is a modified human IL-2, and the IL-2R is a human IL-2R. In some embodiments, the modified IL-2 binds a human IL-2R with an affinity at least 2-fold, 3-fold, 4-fold, 5-fold, 6-fold, 7-fold, 8-fold, 9-fold, at least 10-fold, at least 20-fold, at least 30-fold, at least 50-fold, or at least 100-fold lower than the affinity of human wild type IL-2 for the IL-2R.

In various embodiments, the modified IL-2 comprises at least one substitution at least one amino acid position selected from T3, H16, E61, P65, D84, and C125. In some embodiments, the modified IL-2 comprises substitutions at amino acid positions T3, H16, E61, P65, D84, and C125. In some embodiments, the modified IL-2 comprises substitutions at amino acid positions T3, H16, P65, D84, and C125. In some embodiments, the modified IL-2 comprises substitutions T3A, H16A, E61R, P65R, D84Y, and C125S. In some embodiments, the modified IL-2 comprises substitutions T3A, H116A, P65R, D84S, and C125S. In some embodiments, the modified IL-2 comprises an amino acid sequence at least 90%, 91%, 92%, 93%, 94%, 95%, 96%, 97%, 98%, or 99% identical to SEQ ID NO: 31 or 32. In some such embodiments, the modified IL-2 comprises the amino acid sequence of SEQ ID NO: 31. In some such embodiments, the modified IL-2 comprises the amino acid sequence of SEQ ID NO: 32.

In some embodiments, the NKP46-targeted modified IL-2 polypeptide comprises a NKp46-binding VHH, an Fc region, and a modified IL-2. In some such embodiments, the Fc region comprises an amino acid sequence selected from SEQ ID NOs: 44 and 53-89. In some embodiments, the modified IL-2 is fused to the C-terminus of the Fc region, which is in turn fused to the C-terminus of the NKp46-binding VHH. In some embodiments, the NKp46-binding VHH and Fc region are connected by a linker and the Fc region and the modified IL-2 are connected by a linker. In some such embodiments, the linker comprises 1-20 amino acids, preferably 1-20 amino acids predominantly composed of glycine and, optionally, serine. In some embodiments, the linker comprises: Gly-Gly-Gly-Gly (SEQ ID NO: 45), Gly-Gly-Ser-Gly-Gly-Ser (SEQ ID NO: 46), and/or Gly-Gly-Ser-Ser-Gly-Ser (SEQ ID NO: 47). In some embodiments, the Fc region comprises at least one knob or hole mutation. In some such embodiments, the NKp46-targeted modified IL-2 polypeptide is a portion of a complex comprising a second polypeptide, which comprises a NKp46-binding domain and a second Fc region. In some embodiments, the NKP46-targeted modified IL-2 polypeptide comprises the amino acid sequence of SEQ ID NO: 33. In some embodiments, the complex comprises a first polypeptide comprising the amino acid sequence of SEQ ID NO: 34 or 40 and a second polypeptide comprising a NKp46-binding domain and an Fc region. In some such embodiments, the second polypeptide comprises the amino acid sequence of SEQ ID NO: 33 or 35.

In some embodiments, a NKp46-targeted modified IL-2 polypeptide is a complex comprising a first polypeptide and a second polypeptide, wherein the first polypeptide comprises at least one VHH domain that binds NKp46 and a first Fc region, and the second polypeptide comprises a second Fc region and a modified IL-2. In some such embodiments, at least one VHH domain that binds NKp46 comprises a CDR1 comprising the amino acid sequence of SEQ ID NO: 17; a CDR2 comprising the amino acid sequence of SEQ ID NO: 18, 19, 20, or 21; and a CDR3 comprising the amino acid sequence of SEQ ID NO: 22, 23, 24, 25, 26, or 27. In some embodiments, at least one VHH domain, or each VHH domain, is humanized. In some embodiments, at least one VHH domain of the NKp46-targeted modified IL-2 complex comprises an amino acid sequence at least 85%, at least 90%, at least 95%, or at least 99% identical to an amino acid sequence selected from SEQ ID NOs: 1-16 In some such embodiments, at least one VHH domain comprises an amino acid sequence selected from SEQ ID NOs: 1-16. In some embodiments, a VHH domain that binds NKp46 comprises an amino acid sequence selected from SEQ ID NOs: 1-16, wherein the VHH domain comprises a K125D, K125E, or K125R mutation.

In some embodiments, the modified IL-2 of the NKp46-targeted modified IL-2 complex comprises T3A or T3G, H16A, P65R, C125S, and D84S or D84Y mutations relative to a wild type human IL-2 In some embodiments, the modified IL-2 comprises T3A, H16A, P65R, C125S, and D84S mutations. In some embodiments, the modified IL-2 comprises T3A, H16A, E61R, P65R, C125S, and D84Y mutations in some embodiments, the modified IL-2 comprises an amino acid sequence that is at least 90%, at least 95%, at least 97%, at least 98%, at least 99%, or 100% identical to the amino acid sequence of SEQ ID NO 31 or 32 In some embodiments, the modified IL-2 comprises the amino acid sequence of SEQ ID NO: 31.

In some embodiments, the second polypeptide of the NKp46-targeted modified IL-2 complex comprises at least one antigen-binding domain. In some such embodiments, the at least one antigen-binding domain of the second polypeptide is a VHH domain. In some embodiments, the second polypeptide comprises at least one VHH domain that binds NKp46.

In any of the embodiments described herein, the modified IL-2 may be a modified human IL-2. In various embodiments, the amino acid positions of the substitutions correspond to the amino acid positions in SEQ ID NO: 30.

In various embodiments, an Fc region included in a NKp46-targeted modified IL-2 polypeptide is a human Fc region, or is derived from a human Fc region.

In some embodiments, an Fc region included in a NKp46-targeted modified IL-2 polypeptide is derived from a human Fc region, and comprises a three amino acid deletion in the lower hinge corresponding to IgG1 E233, L234, and L235, herein referred to as “Fc xELL.” Fc xELL polypeptides do not engage FcγRs and thus are referred to as “effector silent” or “effector null”, however in some embodiments, xELL Fc regions bind FcRn and therefore have extended half-life and transcytosis associated with FcRn mediated recycling.

In some embodiments, the Fc region included in a NKp46-targeted modified IL-2 polypeptide is derived from a human Fc region and comprises mutations M252Y and M428V, herein referred to as “Fc-YV”. In some embodiments, such mutations enhance binding to FcRn at the acidic pH1 of the endosome (near 6.5), while losing detectable binding at neutral pH (about 7.2), allowing for enhanced FcRn mediated recycling and extended half-life.

In some embodiments, the Fc region included in a NKp46-targeted modified IL-2 polypeptide is derived from a human Fc region and comprises mutations designed for heterodimerization, herein referred to as “knob” and “hole”. In some embodiments, the “knob” Fc region comprises the mutation T366W. In some embodiments, the “hole” Fc region comprises mutations T366S, L368A, and Y407V. In some embodiments, Fc regions used for heterodimerization comprise additional mutations, such as the mutation S354C on a first member of a heterodimeric Fc pair that forms an asymmetric disulfide with a corresponding mutation Y349C on the second member of a heterodimeric Fc pair. In some embodiments, one member of a heterodimeric Fc pair comprises the modification H435R or H435K to prevent protein A binding while maintaining FcRn binding. In some embodiments, one member of a heterodimeric Fc pair comprises the modification H435R or H435K, while the second member of the heterodimeric Fc pair is not modified at 1-435 In various embodiments, the hold Fc region comprises the modification H435R or H435K (referred to as “hole-R” in some instances when the modification is H435R), while the knob Fc region does not. In some instances, the hole-R mutation improves purification of the heterodimer over homodimeric hole Fc regions that may be present.

In some embodiments, the Fe region included in a NKp46-targeted modified IL-2 polypeptide is derived from a human Fc region and lacks the C-terminal lysine residue. In some embodiments, the Fc region included in a NKp46-targeted modified IL-2 polypeptide is derived from a human Fc region and comprises the C-terminal lysine residue.

Nonlimiting exemplary Fc regions that may be used in a NKp46-targeted modified IL-2 polypeptide include Fc regions comprising the amino acid sequences of SEQ ID NOs: 44 and 53 to 89. In some embodiments, a NKp46-targeted modified IL-2 polypeptide includes an Fc region comprising an amino acid sequence selected from SEQ ID NOs: 44, 56, 57, 63 to 74, and 80 to 89. In some embodiments, a NKp46-targeted modified IL-2 polypeptide includes an Fc region comprising an amino acid sequence selected from SEQ ID NOs: 44 and 63 to 74.

In some embodiments, the first polypeptide comprises the amino acid sequence of SEQ ID NO: 34 and the second polypeptide comprises the amino acid sequence of SEQ ID NO. 33 In some embodiments, the first polypeptide comprises the amino acid sequence of SEQ ID NO: 40 and the second polypeptide comprises the amino acid sequence of SEQ ID NO: 33. In some embodiments, the amino acid sequence of first polypeptide consists of the amino acid sequence of SEQ ID NO: 34 and the amino acid sequence of the second polypeptide consists of the amino acid sequence of SEQ ID NO. 33 In some embodiments, the amino acid sequence of the first polypeptide consists the amino acid sequence of SEQ ID NO: 40 and the amino acid sequence of the second polypeptide consists of the amino acid sequence of SEQ ID NO: 33 In some embodiments, the first polypeptide comprises the amino acid sequence of SEQ ID NO: 42 and the second polypeptide comprises the amino acid sequence of SEQ ID NO: 41. In some embodiments, the first polypeptide comprises the amino acid sequence of SEQ ID NO: 43 and the second polypeptide comprises the amino acid sequence of SEQ ID NO: 41. In some embodiments, the amino acid sequence of first polypeptide consists of the amino acid sequence of SEQ ID NO: 42 and the amino acid sequence of the second polypeptide consists of the amino acid sequence of SEQ ID NO: 41. In some embodiments, the amino acid sequence of the first polypeptide consists the amino acid sequence of SEQ ID NO: 43 and the amino acid sequence of the second polypeptide consists of the amino acid sequence of SEQ ID NO: 41.

### Exemplary Activities of NKp46-Targeted Modified IL-2 Polypeptides

In various embodiments, the NKp46-targeted modified IL-2 polypeptides provided herein stimulate NK cells in vitro and/or in vivo. Stimulation or activity of NK cells in vitro and/or in vivo may be determined, in some embodiments, using the methods provided in the Examples herein. In some embodiments, the NK cell stimulating activity of the modified IL-2 is greater and/or more specifically targeted to NK cells when fused to a NKp46-binding VHH than when used alone. In some embodiments, toxicity of IL-2 is reduced by specifically targeting it to NK cells. In some embodiments, the NKp46-targeted modified IL-2 polypeptides provided herein increase NK cell activation and/or proliferation in vitro and/or in vivo. In some embodiments, the NKp46-targeted modified IL-2 polypeptides provided herein stimulate activation and/or proliferation of NK cells in vivo, and accordingly, the NKp46-targeted modified IL-2 polypeptides may be used in a method of treating cancer or an infectious disease in some embodiments, the NKp46-targeted modified IL-2 polypeptides provided herein upregulate effector proteins such as granzyme B, perforin, or interferon gamma.

In various embodiments, the NKp46-targeting modified IL-2 polypeptides provided herein are agonists of IL-2R activity. Agonist activity may be determined, in some embodiments, using the methods provided in the Examples herein, such as using 293F cells or similar cells. In some embodiments, the NKp46-targeted modified IL-2 polypeptides provided herein are agonists of IL-2R activity when targeted to NK cells, but show little or no agonist activity in the absence of targeting.

In some embodiments, the NKp46-targeted modified IL-2 polypeptides provided herein increase proliferation of NK cells in vitro and/or in vivo. In some such embodiments, the NK cells are activated NK cells. In some embodiments, a NKp46-targeted modified IL-2 polypeptide provided herein increases activated NK cells proliferation in vitro. In some embodiments, the NKp46-targeted modified IL-2 polypeptide increases activated NK cells proliferation by at least 1.5-fold, at least 2-fold, at least 3-fold, or by at least 5-fold relative to NK cell proliferation in the absence of the polypeptide. In some embodiments, the polypeptide increases proliferation of activated NK cells by at least 1.5-fold, at least 2-fold, at least 3-fold, or by at least 5-fold and does not substantially increase the proliferation of inactive NK cells, relative to the proliferation observed in the absence of the polypeptide.

The increase in proliferation of activated NK cells may be determined by any method in the art. A nonlimiting exemplary assay is as follows. NK cells may be isolated from one or more healthy human donors and/or from one or more human donors having a particular disease or disorder. The NK cells are stained, then contacted with a polypeptide comprising a modified IL-2, such as a NKp46-targeted modified IL-2 polypeptide, and then analyzed by FACS. Loss of staining indicates proliferation. In some embodiments, an increase in NK cell proliferation is determined as an average from a set of experiments or from pooled NK cells, such as by measuring proliferation of NK cells isolated from different human donors. In some embodiments, an increase in NK cell proliferation is determined as an average from experiments carried out using NK cells from at least five or at least ten different healthy donors, or from a pool of NK cells from at least five or at least ten different healthy donors. In some embodiments, an increase in NK cell proliferation is determined as an average from experiments carried out using NK cells from at least five or at least ten different donors having a particular disease or disorder, or from a pool of NK cells from at least five or at least ten different donors having a particular disease or disorder.

In some embodiments, the NKp46-targeted modified IL-2 polypeptides provided herein increase the cytotoxicity of NK cells in vitro and/or in vivo. In some embodiments, a NKp46-targeted modified IL-2 polypeptide provided herein increases antibody-dependent cellular cytotoxicity (ADCC). In some embodiments, a NKp46-targeted modified IL-2 polypeptide provided herein increases ADCC in vitro. In some embodiments, the NKp46-targeted modified IL-2 polypeptide increases ADCC by at least 1.5-fold, at least 2-fold, at least 3-fold, or by at least 5-fold relative to ADCC in the absence of the NKp46-targeted modified IL-2 polypeptide.

In some embodiments, the NKp46-targeted modified IL-2 polypeptides provided herein increase pSTAT5 expression specifically in NK cells in vitro and/or in vivo. pSTAT5 expression indicates NK cell activation. In some embodiments, a NKp46-targeted modified IL-2 polypeptide provided herein increases pSTAT5 expression in NK cells in vitro. In some embodiments, the NKp46-targeted modified IL-2 polypeptide increases pSTAT5 expression on NK cells by at least 1.5-fold, at least 2-fold, at least 3-fold, or by at least 5-fold relative to pSTAT5 expression in the absence of the polypeptide. The increase in pSTAT5 expression in NK cells may be determined by any method in the art, such as for example, the methods provided in the Examples herein.

In some embodiments, the NKp46-targeted modified IL-2 polypeptides provided herein reduce or attenuate suppressive activity of regulatory T cells (Tregs). In some embodiments, the NKp46-targeted modified IL-2 polypeptides reduce Treg suppressive activity on NK cells by at least 10%, at least 20%, at least 30%, or by at least 50%. The decrease in Treg suppressive activity on NK cells may be determined by any method in the art. In some embodiments, the NKp46-targeted modified IL-2 polypeptides provided herein increase NK cell activation and proliferation in the presence of Treg cells, for example, compared to NK cell activation and proliferation in the presence of Treg cells but the absence of a NKp46-targeted modified IL-2 polypeptide provided herein.

### Polypeptide Expression and Production

Nucleic acid molecules comprising polynucleotides that encode a NKp46-targeted modified IL-2 polypeptide are provided. In some embodiments, the nucleic acid molecule may also encode a leader sequence that directs secretion of the NKp46-targeted modified IL-2 polypeptide, which leader sequence is typically cleaved such that it is not present in the secreted polypeptide. The leader sequence may be a native heavy chain (or VHH) leader sequence, or may be another heterologous leader sequence.

Nucleic acid molecules can be constructed using recombinant DNA techniques conventional in the art. In some embodiments, a nucleic acid molecule is an expression vector that is suitable for expression in a selected host cell.

Vectors comprising nucleic acids that encode the NKp46-targeted modified IL-2 polypeptides described herein are provided. Such vectors include, but are not limited to, DNA vectors, phage vectors, viral vectors, retroviral vectors, etc. In some embodiments, a vector is selected that is optimized for expression of polypeptides in a desired cell type, such as CHO or CHO-derived cells, or in NSO cells. Exemplary such vectors are described, for example, in Running Deer el al., Biotechnol. Prog 20.880-889 (2004).

In some embodiments, a NKp46-targeted modified IL-2 polypeptide may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells (Crucell), and NSO cells. In some embodiments, the NKp46-targeted modified IL-2 polypeptides may be expressed in yeast. See, e.g., U.S. Publication No. US 2006/0270045 A1. In some embodiments, a particular eukaryotic host cell is selected based on its ability to make desired post-translational modifications to the polypeptide. For example, in some embodiments, CHO cells produce polypeptides that have a higher level of sialylation than the same polypeptide produced in 293 cells.

Introduction of one or more nucleic acids (such as vectors) into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

Host cells comprising any of the nucleic acids or vectors described herein are also provided. In some embodiments, a host cell that expresses a NKp46-targeted modified IL-2 polypeptide described herein is provided. The NKp46-targeted modified IL-2 polypeptides expressed in host cells can be purified by any suitable method. Such methods include, but are not limited to, the use of affinity matrices or hydrophobic interaction chromatography. Suitable affinity ligands include the ROR1 ECD and agents that bind Fc regions. For example, a Protein A, Protein G, Protein A/G, or an antibody affinity column may be used to bind the Fc region and to purify a NKp46-targeted modified IL-2 polypeptide that comprises an Fc region. Hydrophobic interactive chromatography, for example, a butyl or phenyl column, may also suitable for purifying some polypeptides such as antibodies. Ion exchange chromatography (for example anion exchange chromatography and/or cation exchange chromatography) may also suitable for purifying some polypeptides such as antibodies. Mixed-mode chromatography (for example reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

In some embodiments, the NKp46-targeted modified IL-2 polypeptide is produced in a cell-free system. Nonlimiting exemplary cell-free systems are described, for example, in Sitaraman et al, Methods Mol. Biol. 498: 229-44 (2009); Spirin, Trends Biotechnol. 22: 538-45 (2004); Endo et al., Biotechnol. Adv. 21: 695-713 (2003).

In some embodiments, NKp46-targeted modified IL-2 polypeptides prepared by the methods described above are provided. In some embodiments, the NKp46-targeted modified IL-2 polypeptide is prepared in a host cell. In some embodiments, the NKp46-targeted modified IL-2 polypeptide is prepared in a cell-free system. In some embodiments, the NKp46-targeted modified IL-2 polypeptide is purified. In some embodiments, a cell culture media comprising a NKp46-targeted modified IL-2 polypeptide is provided.

In some embodiments, compositions comprising antibodies prepared by the methods described above are provided. In some embodiments, the composition comprises a NKp46-targeted modified IL-2 polypeptide prepared in a host cell. In some embodiments, the composition comprises a NKp46-targeted modified IL-2 polypeptide prepared in a cell-free system. In some embodiments, the composition comprises a purified NKp46-targeted modified IL-2 polypeptide.

### Exemplary Methods of Treating Diseases Using NKp46-Targeted Modified IL-2 Polypeptides

In some embodiments, methods of treating disease in an individual comprising administering a NKp46-targeted modified IL-2 polypeptide are provided. In some embodiments, methods for treating cancer or an infectious disease in an individual are provided. In some embodiments, methods for increasing NK-cell proliferation in an individual comprising administering a NKp46-targeted modified IL-2 polypeptide are provided. In some embodiments, methods for enhancing the ADCC activity of a therapeutic antibody in an individual comprising administering a NKp46-targeted modified IL-2 polypeptide in combination with a therapeutic antibody are provided. In some embodiments, methods for enhancing the cytotoxic capacity of NK cells in an individual comprising administering a NKp46-targeted modified IL-2 polypeptide alone or in combination with a therapeutic antibody are provided. In some embodiments, methods to overcome chemotherapeutic suppression of NK cell activity in an individual comprising administering a NKp46-targeted modified IL-2 polypeptide before, during, or after treatment with a chemotherapeutic agent are provided. In some embodiments, methods for enhancing the ADCC activity of a therapeutic antibody in an individual undergoing chemotherapy comprising administering a NKp46-targeted modified IL-2 polypeptide in combination with a therapeutic antibody before, during, or after treatment with a chemotherapeutic agent are provided.

The method comprises administering to the individual an effective amount of a NKp46-targeted modified IL-2 polypeptide provided herein.

In some embodiments, the NKp46-targeted modified IL-2 polypeptide is used to redirect NK-mediated cytotoxicity. In some such embodiments, the NKp46-targeted modified IL-2 polypeptide also comprises a binding domain that binds a cytotoxic T cell or another NK cell antigen. The binding domain may be, in some embodiments, a VHH domain or an antibody binding domain comprising a heavy chain variable region and a light chain variable region, such as a VH/VL, scFv, Fab fragment, etc. In some embodiments, the NKp46-targeted polypeptide comprises a binding domain binds CD3, T-cell receptor (TCR) α, TCRβ, CD28, CD16, CD32A, CD64, CD89, or NKG2D.

In some such embodiments, the NKp46-targeted modified IL-2 polypeptide also comprises a binding domain that binds a cancer cell. In some embodiments, the binding domain that binds a cancer cell redirects NK-mediated cytotoxicity towards the cancer cell.

In some embodiments, the NKp46-targeted modified IL-2 polypeptide is linked to a cytotoxic agent to form an immunoconjugate. Various cytotoxic agents used in immunoconjugates are known in the art, and include, but are not limited to, calicheamicins, auristatins, dolastatins, tubulicins, maytansinoids, cryptophycins, duocarmycins, esperamicins, pyrrolobenzodiazepines, and enediyne antibiotics. In some embodiments, the NKp46-targeted polypeptide comprises a binding domain binds CD3, T-cell receptor (TCR) α, TCRβ, CD28, CD16, CD32A, CD64, CD89, or NKG2D.

Methods of treatment described herein may be in humans or animals. In some embodiments, methods of treating humans are provided.

Nonlimiting exemplary cancers that may be treated with NKp46-targeted modified IL-2 polypeptides or cells expressing NKp46-targeted modified IL-2 polypeptides provided herein include, but are not limited to, basal cell carcinoma, biliary tract cancer; bladder cancer; bone cancer; brain and central nervous system cancer; breast cancer; cancer of the peritoneum; cervical cancer; choriocarcinoma; colon and rectum cancer, connective tissue cancer; cancer of the digestive system; endometrial cancer; esophageal cancer; eye cancer; cancer of the head and neck; gastric cancer (including gastrointestinal cancer); glioblastoma; hepatic carcinoma, hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma, rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; acute myeloid leukemia (AML), chronic lymphocytic leukemia (CLL); acute lymphoblastic leukemia (ALL); Hairy cell leukemia; chronic myeloblastic leukemia.

The NKp46-targeted modified IL-2 polypeptides can be administered as needed to subjects. Determination of the frequency of administration can be made by persons skilled in the art, such as an attending physician based on considerations of the condition being treated, age of the subject being treated, severity of the condition being treated, general state of health of the subject being treated and the like. In some embodiments, an effective dose of a NKp46-targeted modified IL-2 polypeptide is administered to a subject one or more times. In some embodiments, an effective dose of a NKp46-targeted modified IL-2 polypeptide is administered to the subject daily, semiweekly, weekly, every two weeks, once a month, etc. An effective dose of a NKp46-targeted modified IL-2 polypeptide is administered to the subject at least once. In some embodiments, the effective dose of a NKp46-targeted modified IL-2 polypeptide may be administered multiple times, including multiple times over the course of at least a month, at least six months, or at least a year.

In some embodiments, pharmaceutical compositions are administered in an amount effective for treating (including prophylaxis of) cancer or an infectious disease, for enhancing NK-cell cytotoxic capacity, for increasing NK-cell proliferation or activation, and/or to overcome chemotherapeutic suppression of NK cell activity. The therapeutically effective amount is typically dependent on the weight of the subject being treated, his or her physical or health condition, the extensiveness of the condition to be treated, or the age of the subject being treated. In general, antibodies may be administered in an amount in the range of about 0.05 mg/kg body weight to about 100 mg/kg body weight per dose.

In some embodiments, NKp46-targeted modified IL-2 polypeptides can be administered in vivo by various routes, including, but not limited to, intravenous, intra-arterial, parenteral, intraperitoneal or subcutaneous. The appropriate formulation and route of administration may be selected according to the intended application.

In some embodiments, a therapeutic treatment using a NKp46-targeted modified IL-2 polypeptide is achieved by targeting a NK cell to a NKp46-expressing tumor cell. In some embodiments, therapeutic treatment using a NKp46-targeted modified IL-2 polypeptide is achieved by increasing NK cell proliferation and/or activation. In some embodiments, therapeutic treatment using a NKp46-targeted modified IL-2 polypeptide is achieved by increasing the cytotoxic capacity of NK cells.

### Pharmaceutical Compositions

In some embodiments, compositions comprising NKp46-targeted modified IL-2 polypeptides are provided in formulations with a wide variety of pharmaceutically acceptable carriers (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe el al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In some embodiments, a pharmaceutical composition comprises a NKp46-targeted modified IL-2 polypeptide at a concentration of at least 10 mg/mL.

### Combination Therapy

NKp46-targeted modified IL-2 polypeptides can be administered alone or in combination with other modes of treatment, such as other anti-cancer agents. They can be provided before, substantially contemporaneous with, or after other modes of treatment (i.e., concurrently or sequentially). In some embodiments, the method of treatment described herein can further include administering: radiation therapy, chemotherapy, vaccination, targeted tumor therapy, CAR-T (chimeric antigen receptor T-cell) therapy, oncolytic virus therapy, cancer immunotherapy, cytokine therapy, surgical resection, chromatin modification, ablation, cryotherapy, an antisense agent against a tumor target, a siRNA agent against a tumor target, a microRNA agent against a tumor target or an anti-cancer/tumor agent, or a biologic, such as an antibody, cytokine, or receptor extracellular domain-Fc fusion.

In some embodiments, NKp46-targeted modified IL-2 polypeptides are administered before, during or after treatment with a chemotherapeutic agent. In some embodiments, NKp46-targeted modified IL-2 polypeptides are administered in combination with an antibody comprising a binding domain that binds a tumor antigen, non-limiting examples of tumor antigens that may be bound by such domains are provided herein. In some embodiments, NKp46-targeted modified IL-2 polypeptides are administered in combination with an antibody comprising a binding domain that binds a tumor antigen before, during, or after treatment with one or more chemotherapeutic agents. In some embodiments, NKp46-targeted modified IL-2 polypeptides are administered in combination with an antibody comprising a binding domain that binds the tumor antigen BCMA, CD19, CD20, CD38, CD70, EGFR, or HER2. In some embodiments, NKp46-targeted modified IL-2 polypeptides are administered in combination with an antibody comprising a binding domain that binds the tumor antigen BCMA, CD19, CD20, CD38, CD70, EGFR, or HER2, wherein such administration is before, during, or after treatment with a chemotherapeutic agent.

In some embodiments, the NKp46-targeted modified IL-2 polypeptide is administered in combination with one or more chemotherapeutic agent, CAR-T therapy, oncolytic virus therapy, cytokine therapy, and/or agents that target other checkpoint molecules, such as VISTA, gpNMB, B7H3, B7H4, HHLA2, CD73, CTLA4, TIGIT, etc.

In some embodiments, a NKp46-targeted modified IL-2 polypeptide provided herein is given concurrently with a second therapeutic, for example, a PD-1 or PD-L1 therapy. Examples of PD-1/PD-L1 therapy include nivolumab (BMS); pidilizumab (CureTech, CT-011), pembrolizumab (Merck); durvalumab (Medimmune/AstraZeneca); atezolizumab (Genentech/Roche); avelumab (Pfizer); AMP-224 (Amplimmune); BMS-936559; AMP-514 (Amplimmune); MDX-1105 (Merck). TSR-042 (Tesaro/AnaptysBio, ANB-011), STI-A1010 (Sorrento Therapeutics); STI-A1110 (Sorrento Therapeutics); and other agents that are directed against programmed death-1 (PD-1) or programmed death ligand 1 (PD-L1).

In some embodiments, a NKp46-targeted modified IL-2 polypeptide provided herein is given concurrently with an immune stimulatory agent, for example, an agonist of a member of the Tumor Necrosis Factor Receptor Super Family (TNFRSF) or a member the B7 family. Nonlimiting examples of immune stimulatory TNFRSF members include OX40, GITR, 41BB, CD27, and HVEM. Nonlimiting examples of B7 family members include CD28 and ICOS. Thus, in some embodiments, a NKp46-targeted modified IL-2 polypeptide provided herein is given concurrently with an agonist, such as an agonist antibody, of OX40, GITR, 41BB, CD27, HVEM, CD28, and/or ICOS.

In some embodiments, the NKp46-targeted modified IL-2 polypeptide and an additional agent are formulated into a single therapeutic composition, and the NKp46-targeted modified IL-2 polypeptide and additional agent are administered simultaneously. Alternatively, the NKp46-targeted modified IL-2 polypeptide and the additional agent are separate from each other, e.g., each is formulated into a separate therapeutic composition, and the NKp46-targeted modified IL-2 polypeptide and the additional agent are administered simultaneously, or the NKp46-targeted modified IL-2 polypeptide and the additional agent are administered at different times during a treatment regimen. For example, the NKp46-targeted modified IL-2 polypeptide is administered prior to the administration of the additional agent, the NKp46-targeted modified IL-2 polypeptide is administered subsequent to the administration of the additional agent, or the NKp46-targeted modified IL-2 polypeptide and the additional agent are administered in an alternating fashion. The NKp46-targeted modified IL-2 polypeptide and additional agent may be administered in single doses or in multiple doses.

### Nonlimiting Exemplary Methods of Diagnosis and Treatment

In some embodiments, the methods described herein are useful for evaluating a subject and/or a specimen from a subject (e.g. a cancer patient). In some embodiments, evaluation is one or more of diagnosis, prognosis, and/or response to treatment.

In some embodiments, the methods described herein comprise evaluating a presence, absence, or level of a protein. In some embodiments, the methods described herein comprise evaluating a presence, absence, or level of expression of a nucleic acid. The compositions described herein may be used for these measurements. For example, in some embodiments, the methods described herein comprise contacting a specimen of the tumor or cells cultured from the tumor with a therapeutic agent as described herein.

In some embodiments, the evaluation may direct treatment (including treatment with the antibodies described herein). In some embodiments, the evaluation may direct the use or withholding of adjuvant therapy after resection. Adjuvant therapy, also called adjuvant care, is treatment that is given in addition to the primary, main or initial treatment. By way of non-limiting example, adjuvant therapy may be an additional treatment usually given after surgery where all detectable disease has been removed, but where there remains a statistical risk of relapse due to occult disease. In some embodiments, the polypeptides are used as an adjuvant therapy in the treatment of a cancer. In some embodiments, the polypeptides are used as the sole adjuvant therapy in the treatment of a cancer. In some embodiments, the polypeptides described herein are withheld as an adjuvant therapy in the treatment of a cancer. For example, if a patient is unlikely to respond to an antibody described herein or will have a minimal response, treatment may not be administered in the interest of quality of life and to avoid unnecessary toxicity from ineffective chemotherapies. In such cases, palliative care may be used.

In some embodiments the polypeptides are administered as a neoadjuvant therapy prior to resection. In some embodiments, neoadjuvant therapy refers to therapy to shrink and/or downgrade the tumor prior to any surgery. In some embodiments, neoadjuvant therapy means chemotherapy administered to cancer patients prior to surgery. In some embodiments, neoadjuvant therapy means an antibody is administered to cancer patients prior to surgery. Types of cancers for which neoadjuvant chemotherapy is commonly considered include, for example, breast, colorectal, ovarian, cervical, bladder, and lung. In some embodiments, the polypeptides are used as a neoadjuvant therapy in the treatment of a cancer. In some embodiments, the use is prior to resection.

In some embodiments, the tumor microenvironment contemplated in the methods described herein comprises or is one or more of tumor vasculature; tumor-infiltrating lymphocytes; fibroblast reticular cells; endothelial progenitor cells (EPC); cancer-associated fibroblasts; pericytes; other stromal cells; components of the extracellular matrix (ECM), dendritic cells; antigen presenting cells; T-cells; regulatory T-cells; NK cells; macrophages; other lymphoid cells; neutrophils; and other immune cells located proximal to a tumor.

### Kits

Also provided are articles of manufacture and kits that include any of NKp46-targeted modified IL-2 polypeptides as described herein, and suitable packaging. In some embodiments, the invention includes a kit with (i) a NKp46-targeted modified IL-2 polypeptide, and (ii) instructions for using the kit to administer the NKp46-targeted modified IL-2 polypeptide to an individual.

Suitable packaging for compositions described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed. Instructions supplied in the kits of the invention are typically written instructions on a label or package insert (e.g., a paper sheet included in the kit), but machine-readable instructions (e.g., instructions carried on a magnetic or optical storage disk) are also acceptable. The instructions relating to the use of the antibodies generally include information as to dosage, dosing schedule, and route of administration for the intended treatment or industrial use. The kit may further comprise a description of selecting an individual suitable or treatment.

The containers may be unit doses, bulk packages (e.g., multi-dose packages) or sub-unit doses. For example, kits may also be provided that contain sufficient dosages of molecules disclosed herein to provide effective treatment for an individual for an extended period, such as about any of a week, 2 weeks, 3 weeks, 4 weeks, 6 weeks, 8 weeks, 3 months, 4 months, 5 months, 6 months, 7 months, 8 months, 9 months, or more. Kits may also include multiple unit doses of molecules and instructions for use and packaged in quantities sufficient for storage and use in pharmacies, for example, hospital pharmacies and compounding pharmacies. In some embodiments, the kit includes a dry (e.g., lyophilized) composition that can be reconstituted, resuspended, or rehydrated to form generally a stable aqueous suspension of antibody.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: NKp46 Single-Domain Antibodies

Single domain antibodies targeting human NKp46 were generated via immunization of llamas and alpacas with a recombinant version of the human NKp46 extracellular domain. The amino acid sequences of the NKp46 VHH domains are provided in the Table of Certain Sequences provided below. It is provided that the lysine at residue 125 (K125) in any of the disclosed VHH domains may be substituted with an aspartate (K125D), a glutamate (K125E), or an arginine (K125R). The VHH designated hz5D7v17 (SEQ ID NO: 15) comprises an arginine (R) at residue 125 (shown bolded and underlined in the Table of Certain Sequences).

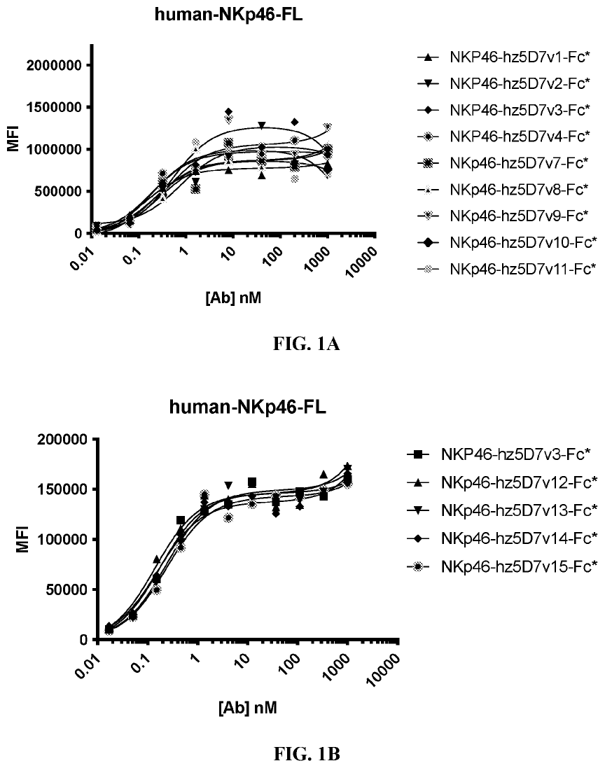

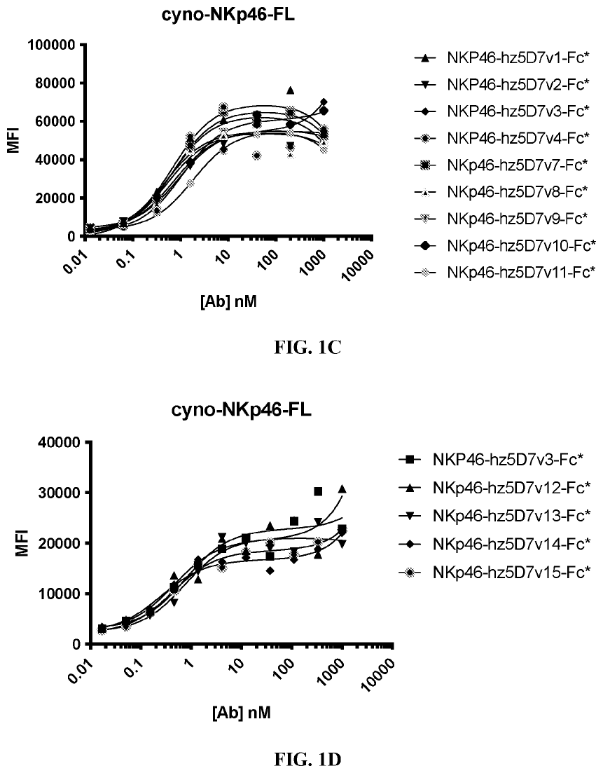

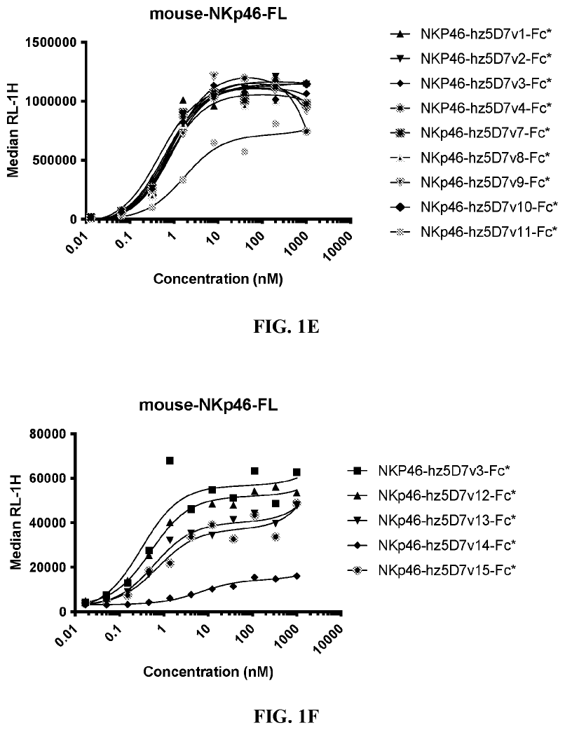

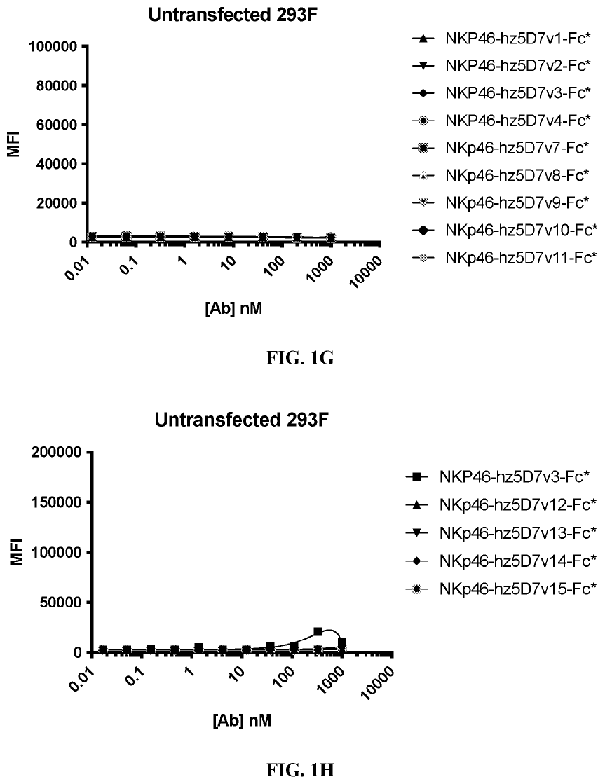

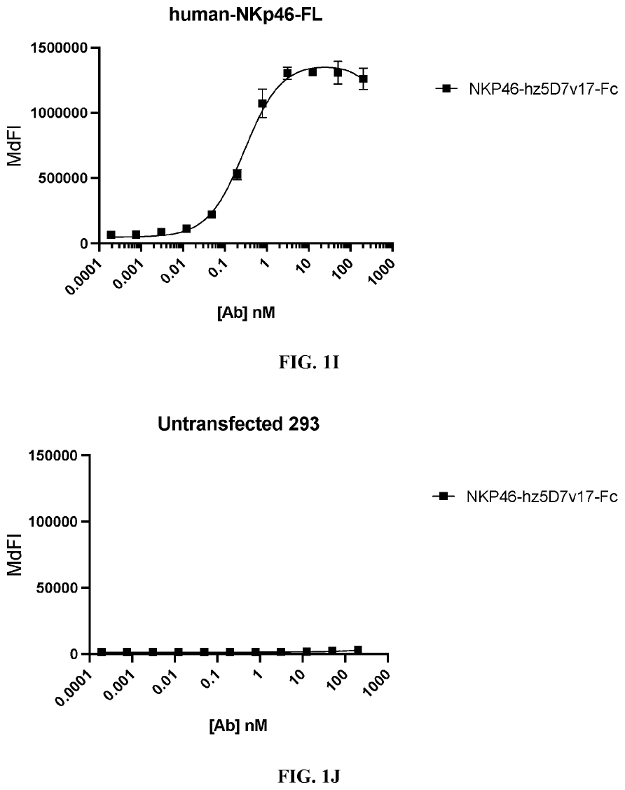

Binding of NKp46-binding polypeptides formatted as VHH-Fc fusion proteins was assessed by flow cytometry. An IgG1-Fc lacking a hinge, or a homodimeric Fc (FIG. 1I-1J) was used (the Fc lacking a hinge is annotated as Fc*). HEK293F cells were transiently transfected with a plasmid encoding full-length human NKp46, cynomolgus NKp46, or mouse NKp46, followed by an IRES and GFP. Transfected cells expressing NKp46 and GFP were used to measure binding of the polypeptides. The transfected cells were plated in a 96-well plate at 30,000 cells per well in FACS buffer (PBS, 1% BSA, 0.1% NaN₃, pH 7.4). Untransfected HEK293F cells were used as a NKp46-negative control and plated at 30,000 cells per well in a separate plate. Test polypeptides were then diluted to two times the final concentration of 1000 nM, and a 3-, 4-, or 5-fold, serial dilution was made. A final column was left with only FACS buffer as a secondary-only control. Test article dilutions were added to an equal volume of cells, and assay plates were incubated for 30 minutes at 4° C. After washing twice with 150 μL. of FACS buffer per well, the cells were resuspended in FACS buffer with α-hFc-647 secondary diluted 1:1000-2000. Assay plates were then left to incubate at 4° C. for 20-30 minutes. After one additional wash with 150 μL of FACS buffer, bound antibody was detected by flow cytometry. Flow cytometric detection was performed on an Intellicyt iQue Plus or Accuri iQue. NKp46 transfected cells were gated on as GFP positive, and polypeptide binding was measured as median fluorescence at 647 nm. The data was plotted and analyzed using GraphPad Prism analysis software, and the results are shown in the tables below and in FIG. 1.

TABLE 3
Binding on HEK-293-FL transfected with human NKp46

| Fusion Protein | Bmax (MFI) | K_{d} (nM) | SEQ ID NOs. |
|---|---|---|---|
| NKp46-5D7-Fc* | 1043043 | 0.1716 | 1, 53 |
| NKp46-hz5D7v1-Fc* | 790331 | 0.1391 | 2, 53 |
| NKp46-hz5D7v2-Fc* | 938268 | 0.7624 | 3, 53 |
| NKp46-hz5D7v3-Fc* | 1274160 | 0.5106 | 4, 53 |
| NKp46-hz5D7v4-Fc* | 979738 | 0.189 | 5, 53 |
| NKp46-hz5D7v7-Fc* | 866073 | 0.2303 | 6, 53 |
| NKp46-hz5D7v8-Fc* | 930045 | 0.2032 | 7, 53 |
| NKp46-hz5D7v9-Fc* | 1050045 | 0.3922 | 8, 53 |
| NKp46-hz5D7v10-Fc* | 883558 | 0.2685 | 9, 53 |
| NKp46-hz5D7v11-Fc* | 1049076 | 0.1887 | 10, 53 |

TABLE 4
Binding on HEK-293-FL transfected with human NKp46

| Fusion Protein | Bmax (MFI) | K_{d} (nM) | SEQ ID NOs. |
|---|---|---|---|
| NKp46-hz5D7v3-Fc* | 148738 | 0.1772 | 4, 53 |
| NKp46-hz5D7v12-Fc* | 149338 | 0.1403 | 11, 53 |
| NKp46-hz5D7v13-Fc* | 149680 | 0.2336 | 12, 53 |
| NKp46-hz5D7v14-Fc* | 138119 | 0.1582 | 13, 53 |
| NKp46-hz5D7v15-Fc* | 143134 | 0.2368 | 14, 53 |

TABLE 5
Binding on HEK-293-FL transfected with cyno NKp46

| Fusion Protein | Bmax (MFI) | K_{d} (nM) | SEQ ID NOs. |
|---|---|---|---|
| NKp46-5D7-Fc* | 58584 | 0.5384 | 1, 53 |
| NKp46-hz5D7v1-Fc* | 67630 | 0.6907 | 2, 53 |
| NKp46-bz5D7v2-Fc* | 51506 | 0.9016 | 3, 53 |
| NKp46-bz5D7v3-Fc* | 51750 | 0.7264 | 4, 53 |
| NKp46-hz5D7v4-Fc* | 55388 | 0.4458 | 5, 53 |
| NKp46-bz5D7v7-Fc* | 64599 | 0.7033 | 6, 53 |
| NKp46-bz5D7v8-Fc* | 53537 | 0.5262 | 7, 53 |
| NKp46-hz5D7v9-Fc* | 61889 | 0.6549 | 8, 53 |
| NKp46-hz5D7v10-Fc* | 58615 | 1.061 | 9, 53 |
| NKp46-hz5D7v11-Fc* | 52344 | 1.843 | 10, 53 |

TABLE 6
Binding on HEK-293-FL transfected with cyno NKp46

| Fusion Protein | Bmax (MFI) | K_{d} (nM) | SEQ ID NOs. |
|---|---|---|---|
| NKp46-hz5D7v3-Fc* | 19752 | 0.773 | 4, 53 |
| NKp46-hz5D7v12-Fc* | 17924 | 0.4548 | 11, 53 |
| NKp46-hz5D7v13-Fc* | 18754 | 0.7484 | 12, 53 |
| NKp46-hz5D7v14-Fc* | 14322 | 0.2516 | 13, 53 |
| NKp46-hz5D7v15-Fc* | 16394 | 0.4281 | 14, 53 |

TABLE 7
Binding on HEK-293-FL transfected with mouse NKp46

| Fusion Protein | Bmax (MFI) | K_{d} (nM) | SEQ ID NOs. |
|---|---|---|---|
| NKp46-5D7-Fc* | 1069378 | 0.4868 | 1, 53 |
| NKp46-hz5D7v1-Fc* | 1173183 | 0.4683 | 2, 53 |
| NKp46-hz5D7v2-Fc* | 1208032 | 0.7292 | 3, 53 |
| NKp46-hz5D7v3-Fc* | 1166480 | 0.6851 | 4, 53 |
| NKp46-hz5D7v4-Fc* | 1272032 | 0.8874 | 5, 53 |
| NKp46-hz5D7v7-Fc* | 1171975 | 0.6129 | 6, 53 |
| NKp46-hz5D7v8-Fc* | 1117364 | 0.6958 | 7, 53 |
| NKp46-hz5D7v9-Fc* | 1217300 | 0.9408 | 8, 53 |
| NKp46-hz5D7v10-Fc* | 1189995 | 0.8201 | 9, 53 |
| NKp46-hz5D7v11-Fc* | 722792 | 1.699 | 10, 53 |

TABLE 8
Binding on HEK-293-FL transfected with mouse NKp46

| Fusion Protein | Bmax (MFI) | K_{d} (nM) | SEQ ID NOs. |
|---|---|---|---|
| NKp46-hz5D7v3-Fc* | 56507 | 0.3126 | 4, 53 |
| NKp46-hz5D7v12-Fc* | 49278 | 0.5127 | 11, 53 |
| NKp46-hz5D7v13-Fc* | 38140 | 0.6037 | 12, 53 |
| NKp46-hz5D7v14-Fc* | 11312 | 6.510 | 13, 53 |
| NKp46-hz5D7v15-Fc* | 34349 | 0.7289 | 14, 53 |

TABLE 9
Binding on HEK-293-FL transfected with human NKp46

| Fusion Protein | Bmax (MFI) | K_{d} (nM) | SEQ ID NOs. |
|---|---|---|---|
| NKp46-hz5D7v17-Fc | 1335271 | 0.2949 | 15, 55 |

As shown in FIGS. 1A-1B, 1I and Tables 3, 4, and 9, the NKp46-targeted polypeptides bound human NKp46 with affinities below 1 nM. FIGS. 1C-1D and Tables 5 and 6 show that the NKp46-targeted polypeptides bound cynomolgus NKp46 with affinities below 2 nM. FIG. 1E-1F and Tables 7 and 8 show that nearly all of the NKp46-targeted polypeptides bound mouse NKp46 with affinities below 2 nM. FIGS. 1G-1H, and 1J show that the polypeptides did not bind to untransfected HEK-293F cells.

### Example 2: Specific IL-2 Signaling Induced by a Polypeptide Comprising a NKp46-Binding VHH and an IL-2 Variant

NKp46-targeted IL-2 activity of a NKp46-targeted modified IL-2 polypeptides cx10454 and cx11314 comprising NKp46-binding VHH domain (hz5D7v12 or hz5D7v17), a heterodimeric knob-in-hole Fc region, and an IL-2 variant fused to the C-terminus of the Fc region was assessed in a phospho-STAT5 assay. As shown in the table below, control proteins included a polypeptide comprising hz5D7v12 and a heterodimeric knob-in-hole Fc region with no IL-2 (cx8411), a polypeptide comprising a non-targeting VHH and an IL-2 variant fused to the C-terminus of a heterodimeric knob-in-hole Fc region (cx9452), and wild-type recombinant IL-2. Increases in levels of phosphorylated STAT5 (pSTAT5) were measured by intracellular flow cytometry as a proximal readout of IL-2 receptor engagement and signaling. Human PBMCs were plated in a 96-well plate at 1,000,000 cells per well in complete growth media (RPMI, 10% FBS, 1% anti-anti). Test polypeptides were then diluted to 2× the final concentration of 100 nM and a 5-fold serial dilution was made. Serial dilutions were added to the cells and incubated for 15 minutes at 37° C. Cells were then fixed in 100 μL of Cytofix fixation buffer (BD) for 30 minutes at 4° C. Cells were then washed once in 200 μL FACS buffer and permeabilized in Perm buffer III (BD Phosflow) for 30 minutes at 4° C. Permeabilized cells were washed a total of three times in 1× Permeabilization Buffer (eBioscience) and then incubated in 1× Permeabilization Buffer containing fluorescently labeled antibodies against CD4 (OKT4, 1:100), CD3 (SP34-2, 1-50), CD16 (3G8, 1:1000), pSTAT5 (SRBCZX, 1:70), CD56 (NCAM16.2, 1:500), and CD8 (RPA-T8, 1:4000) overnight at 4° C. The next day, cells were washed with 150 μL FACS buffer and analyzed using an ACEA Biosciences Novocyte-Quanteon Flow Cytometer. IL-2 signaling was quantified via increases in the frequency and median fluorescence intensity levels of the fluorescently labeled antibody detecting pSTAT5 on NK cells (CD3−CD56^{dim}CD16+ or CD3−CD56^{bright}CD16−, or total NK cells) or CD4 T cells (CD3+CD4+). The data were plotted and analyzed using GraphPad Prism analysis software.

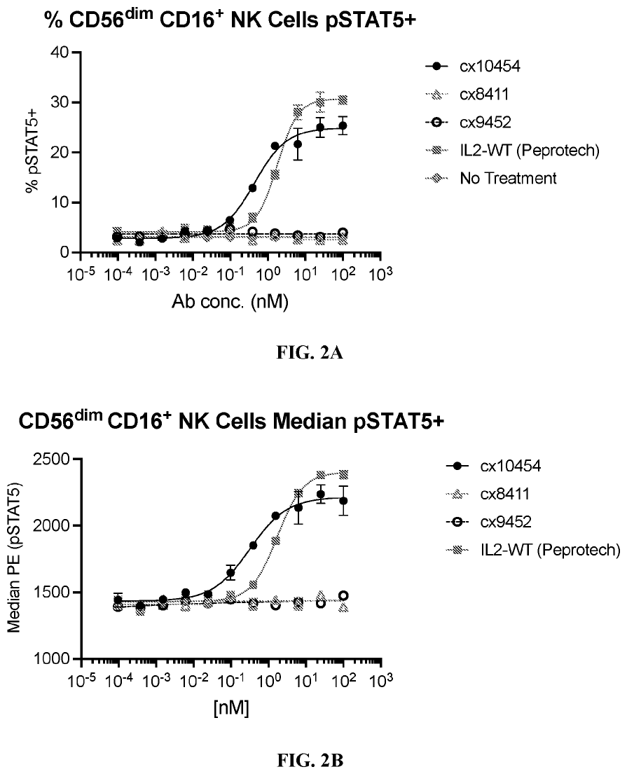

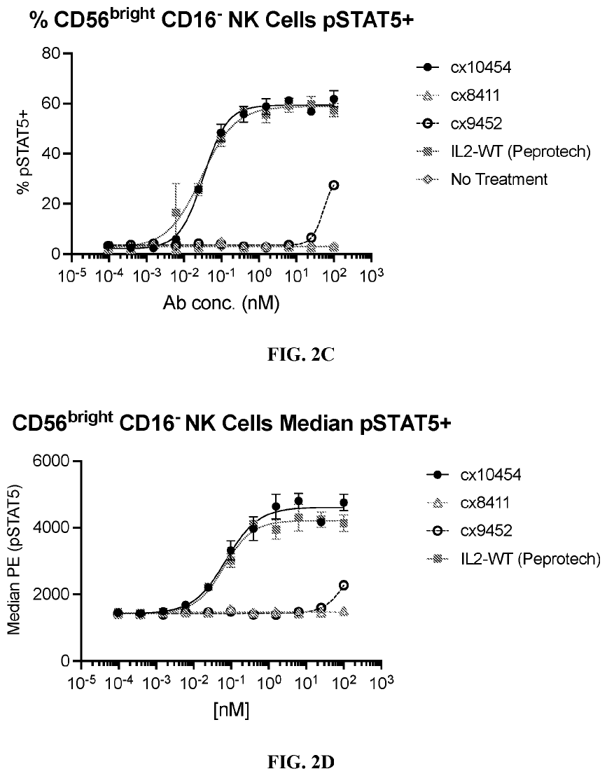

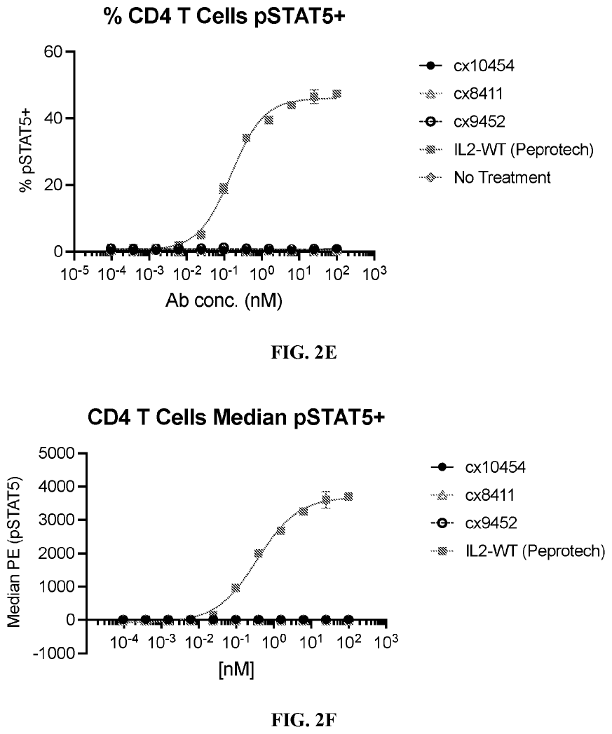

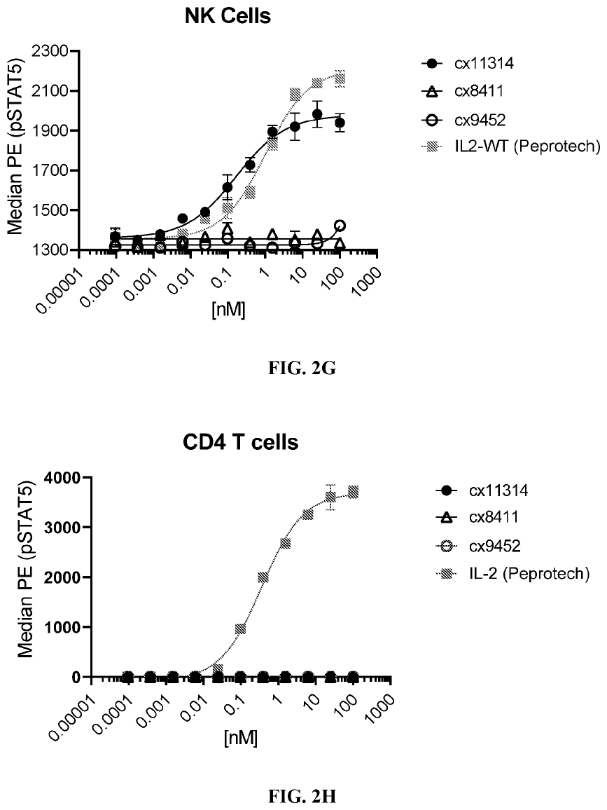

As shown in FIG. 2, NKp46-targeted modified IL-2 polypeptides comprising a NKp46-binding VHH domain and an IL-2 variant fused to the C-terminus of a heterodimeric knob-in-hole Fc region (e.g., cx10454 or cx11314) induced increasing levels of pSTAT5 in a concentration-dependent manner and with an EC₅₀ below 0.4 nM on CD56^{dim}CD16+NK cells (FIGS. 2A-2B) and total NK cells (FIG. 2G), which was lower than the activity of wild type recombinant IL-2. On CD56^{bright}CD16− NK cells the EC₅₀ was below 0.08 nM (FIG. 2C-2D), which was similar to the approximately 0.06 nM EC₅₀ of wild type recombinant IL-2. No detectable increases in pSTAT5 on CD4 T cells were induced by the NKp46-targeted variant IL-2 (FIGS. 2E-2F, 2H-2I). Neither of the control polypeptides induced detectable increases in pSTAT5 levels in any of the cell types, indicating that the attenuated IL-2 required targeting to a cell via a binding domain in order to achieve IL-2 receptor signaling activity.

TABLE 10
Polypeptides

| Polypeptide or complex | SEQ ID NOs |
|---|---|
| hz5D7v12-Fc xELL-Hole; and | 11, 70; and 11, 63, 31 |
| hz5D7v12-Fc xELL-Knob-IL2-T3A-H16A- | (33 and 34) |
| E61R-P65R-D84Y-C125S (cx10454) | |
| hz5D7v17-Fc xELL-Hole; and | 15, 44; and 15, 63, 31 |
| hz5D7v17-Fc xELL-Knob-IL2-T3A-H16A- | (41 and 42) |
| E61R-P65R-D84Y-C125S (cx11314) | |
| hz5D7v12-Fc xELL-Hole; and | 11, 70; and 11, 63 |
| hz5D7v12-Fc xELL-Knob (cx8411) | (35 and 36) |
| Non-targeted VHH-Fc xELL-Hole; and | Non-targeted VHH, 70; |
| Non-targeted VHH-Fc xELL-Knob-IL2-T3A | and Non-targeted VHH, |
| H16A E61R P65R D84Y C125S (cx9452) | 63, 31 (37 and 38) |
| WT IL-2 | 30 |

### Example 3: Enhancement of Antibody-Dependent Cellular Cytotoxicity Induced by Polypeptides Comprising a NKp46-Binding VHH and an IL-2 Variant

The NKp46-targeted IL-2 activity of a NKp46-targeted modified IL-2 polypeptide comprising NKp46-binding VHH domain hz5D7v12, a heterodimeric knob-in-hole Fc region, and an IL-2 variant fused to the C-terminus of the Fc region (cx10454) was further assessed in an antibody-dependent cellular cytotoxicity (ADCC) assay in combination with cetuximab that exhibits ADCC activity. A polypeptide comprising a non-targeting VHH and an IL-2 variant fused to the C-terminus of a heterodimeric knob-in-hole Fc region (cx9452) and wild type recombinant IL-2 were used as controls A431 cells were labeled with CYTO-ID red long-term cell tracer (Enzo) then plated at 10,000 cells per well in 100 μL in a 96-well flat-bottom plate and allowed to adhere for 4 hours. PB MCs obtained from human donors were thawed and tested for NK cell frequency by flow cytometry. 25 μL of Incucyte® Caspase-3/7 Green Dye for Apoptosis (Sartorius) for a final dilution of 1:2000, 25 μL of media or cetuximab at a final concentration of 20 nM or 0.2 nM, 25 μL of media, wild type recombinant IL-2 at a final concentration of 1 nM, or IL-2 variant fusion polypeptides at a final concentration of 1 nM, and 25 μL of human PBMCs adjusted to a concentration of 10 NK cells per 1 A431 cell were added to each well. Cells were allowed to settle at room temperature for 10 minutes, then the plate was placed in an Incucyte imager at 37° C. for 24 hours with image acquisition every 30 minutes. A431 killing was determined by the overlap of Caspase-3/7 and CYTO-ID red, with maximal killing defined by the levels observed when 20 nM cetuximab was used. The data were plotted and analyzed using GraphPad Prism analysis software.

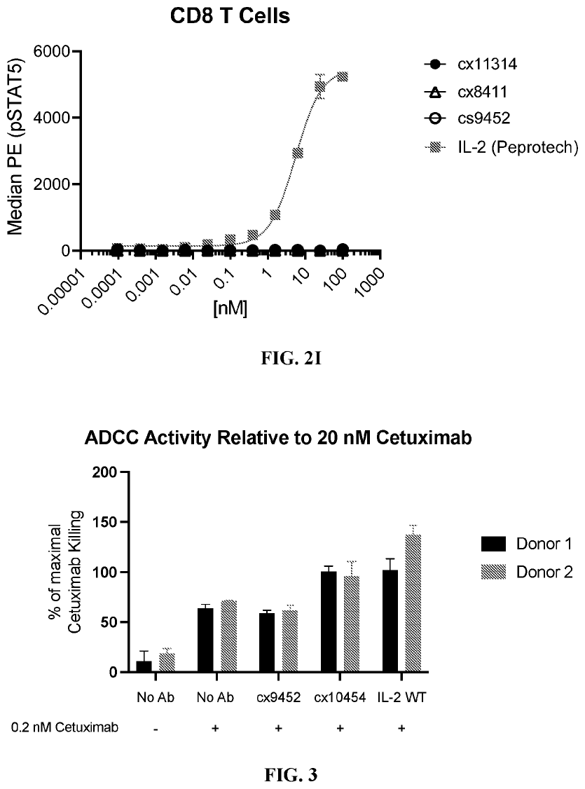

As shown in FIG. 3, the ADCC activity of cetuximab was reduced to approximately 65% of maximal activity when 0.2 nM cetuximab was used. The killing activity of this suboptimal cetuximab dose was not enhanced by a polypeptide comprising the IL-2 variant, a heterodimeric Fc, and non-targeted VHH (cx9452), while both wild type recombinant IL-2 and a NKp46-targeted modified IL-2 polypeptide comprising an IL-2 variant fused to the C-terminus of a heterodimeric Fc and a NKp46-targeted VHH domain (e.g., cx10454) were able to enhance the activity of 0.2 nM cetuximab such that maximal killing was achieved.

In additional studies, target cell killing was assessed using a PBMC ADCC bioassay (Promega). The kit contains target cells expressing a HiBit fusion protein that is released upon cell lysis and generates a luminescence signal upon binding to its complementary polypeptide LgBiT. In this assay, target cells were mixed with the specified test articles (an anti-BCMA antibody, cetuximab (anti-EGFR), trastuzumab (anti-HER2), sequence analogs of: rituximab (anti-CD20), an afucosylated variant of rituximab (anti-CD20), daratumumab (anti-CD38), tafasitamab (anti-CD19), obinutuzumab (anti-CD19), alone or in combination with cx11314) and human PBMC's ratios in a 96-well white U-bottom plate. The assay plate was then incubated in a 37° C. incubator for 5 hours. Subsequently, the detection reagent, which contains the polypeptide LgBiT, was added to each well, and the luminescence was read out on a plate reader. A maximum lysis control, where 100 ug/mL digitonin was added to a well with PBMCs and target cells, and an untreated control was included in each experiment. The % specific lysis was calculated based on the relative light units of each sample alongside the untreated and digitonin controls.

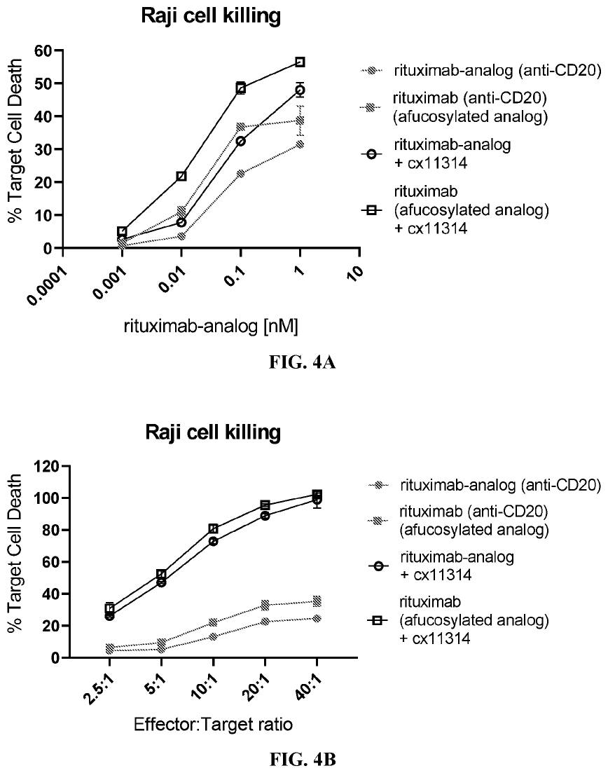

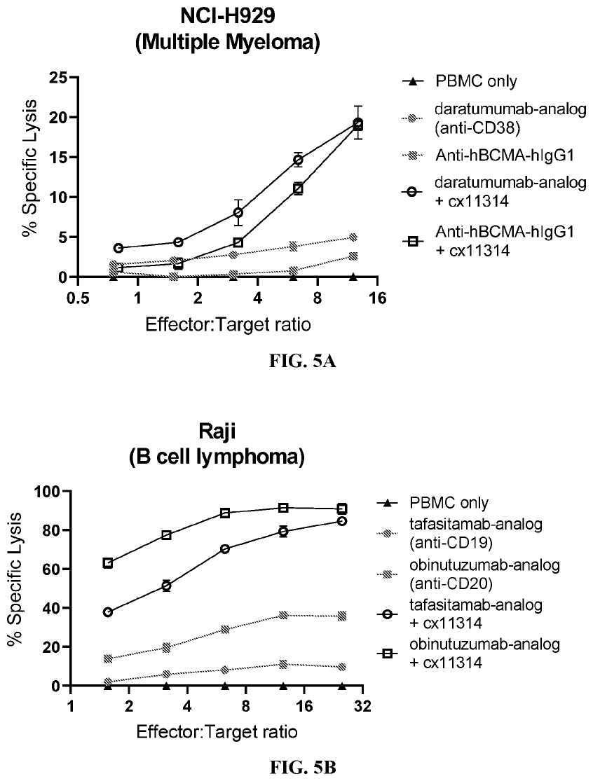

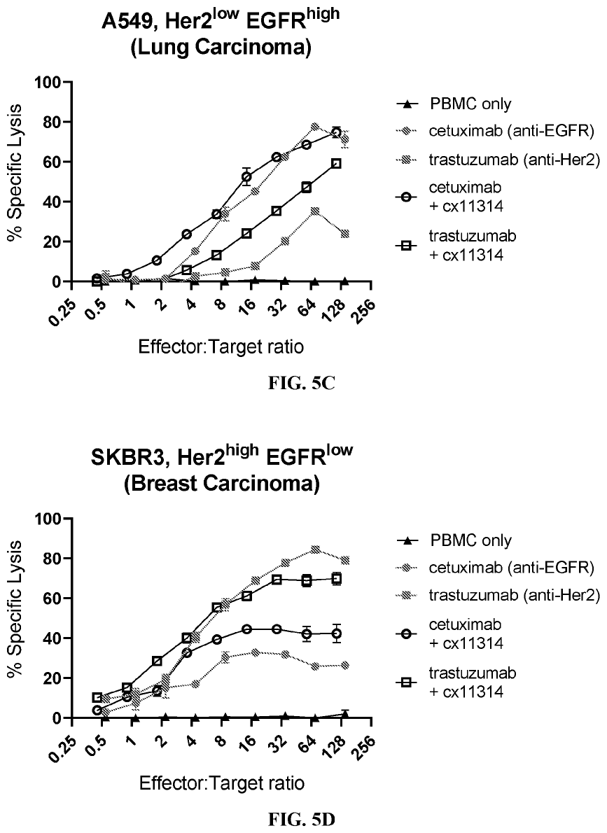

As shown in FIGS. 4 and 5, a NKp46-targeted modified IL-2 polypeptide comprising an IL-2 variant fused to the C-terminus of a heterodimeric Fc and a NKp46-targeted VHH domain (cx11314) greatly enhanced the ADCC activity of various antibodies targeting cell surface antigens, including, CD20 (FIGS. 4A-4B, and 5B), CD19 (FIG. 5B), CD38 (FIG. 5A), BCMA (FIG. 5A), HER2 (FIGS. 5C-5I)), and EGFR (FIGS. 5C-5D).

### Example 4: Cell Expansion of Cynomolgus PBMC Subpopulations Induced by Polypeptides Comprising a NKp46-Binding VHH and an IL-2 Variant

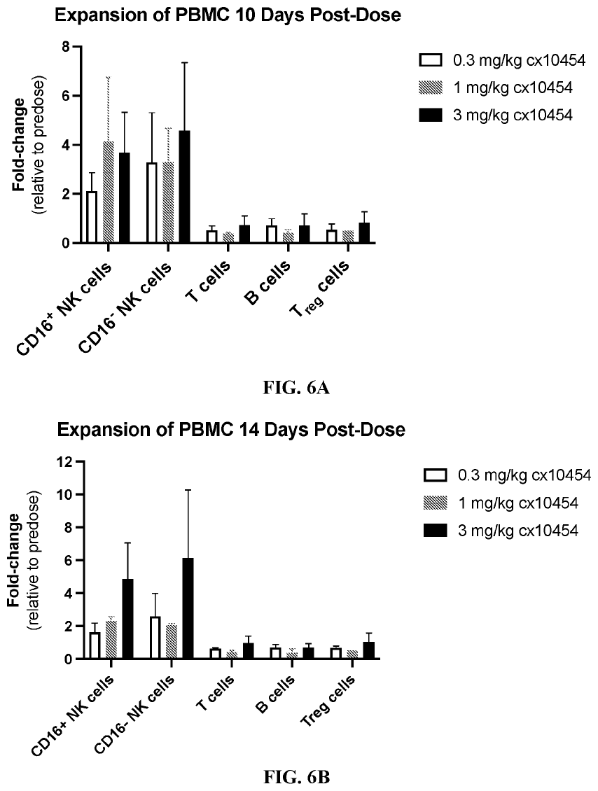

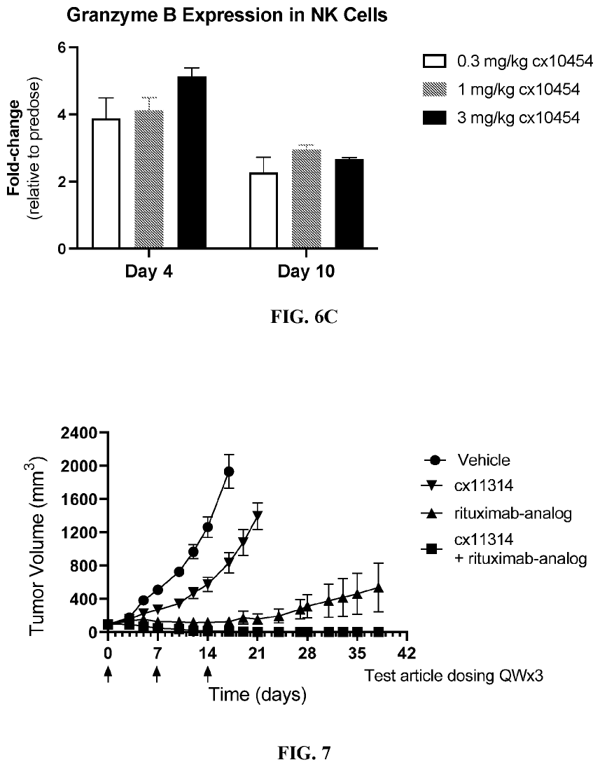

The effects on in vivo cell expansion of a NKp46-targeted modified IL-2 polypeptide cx10454 comprising NKp46-binding VHH domain hz5D7v12, a heterodimeric Fc region, and an IL-2 variant fused to the C-terminus of the Fc region were tested in non-human primates. Cynomolgus monkeys were administered an intravenous bolus injection of the polypeptide at 0.3 mg/kg, 1 mg/kg, or 3 mg/kg. Whole blood samples were collected from the study animals before dosing and at 4, 10, and 14 days post-dosing. PBMC from each time point were isolated using density centrifugation in Lymphoprep (STEMCELL Technologies) and cells were stained with fluorescently labeled cell type-specific antibody combinations. T cells were classified as CD3⁺ cells that did not express the B cell marker CD20. Regulatory T cells (T_{regs}) were defined as CD4⁺ T cells that also expressed CD25 and had reduced levels of CD127 NK cells were defined as CD3⁻ non-T and non-B cells expressing NKG2A and were either positive or negative for CD16. The population staining positive for CD20 was classified as B cells. Fold-change was calculated by dividing the cell count per mL of whole blood 10 days post-dose by the baseline count per mL of whole blood pre-dose Granzyme B expression was measured in the PBMC subpopulations described above using additional fixation, permeabilization, and staining steps. In brief, cells were stained with fluorescently labeled cell type-specific antibody combinations for the cell surface markers, then fixed and permeabilized using the FoxP3 Transcription Factor Staining Buffer Set (eBioscience) Granzyme B was then detected with specific fluorescently labeled antibodies. Flow cytometric detection was performed on an ACEA Biosciences Novocyte-Quanteon Flow Cytometer. The data were plotted and analyzed using GraphPad Prism analysis software. Fold-change was calculated by dividing the granzyme B median fluorescence at days 4 and 10 by the median fluorescence of NK cells at baseline (pre-dosing). The results are shown in Table 11 and FIG. 6A-6C.

TABLE 11
Fold-expansion of PBMC subpopulations

| | 0.3 mg/kg | 1 mg/kg | 3 mg/kg |
|---|---|---|---|
| | Fold-Change | Fold-Change | Fold-Change |
| PBMC | at Day 10, | at Day 10, | at Day 10, |
| Subpopulation | Day 14 | Day 14 | Day 14 |
| CD16⁺ NK Cells | 2.1, 1.6 | 4.1, 2.3 | 3.7, 4.9 |
| CD16⁻ NK Cells | 3.3, 2.6 | 3.3, 2.1 | 4.9, 6.1 |

As shown in FIG. 6A-6B and Table 11, a single dose of a NKp46-targeted modified IL-2 polypeptide comprising NKp46-binding VHH domain hz5D7v12, a heterodimeric Fc region, and an IL-2 variant fused to the C-terminus of the Fc region resulted in NK cell expansion in a dose-dependent manner, with higher expansion occurring at day 10 for the 0.3 mg/kg and 1 mg/kg doses and at day 14 for the 3 mg/kg dose. Greater than 2-fold expansion was seen in both the CD16′ and CD16 NK cell compartments at all dose levels with the greatest fold-expansion seen at the 3 mg/kg dose. No expansion was seen in non-NK cell populations, including T cells, B cells, and T_{reg} cell populations. FIG. 6C shows that the treatment with a single dose of a NKp46-targeted modified IL-2 polypeptide comprising NKp46-binding VHH domain hz5D7v12, a heterodimeric Fc region, and an IL-2 variant fused to the C-terminus of the Fc region also resulted in an increase in NK cell killing capacity as demonstrated by the upregulation of a surrogate marker for cytotoxicity, granzyme B. Expression of granzyme B peaked at 4 days post-dose but remained elevated above pre-dose levels at 10 days post-dose. Maximal granzyme B expression increases were 3.9-fold, 4.1-fold, and 5.1-fold in the 0.3 mg/kg, 1 mg/kg, and 3 mg/kg groups, respectively. These data show that a NKp46-targeted modified IL-2 polypeptide comprising a NKp46-binding VHH domain (e.g., hz5D7v12), a heterodimeric Fc region, and an IL-2 variant fused to the C-terminus of the Fc region specifically induced cell proliferation and activity of NK cell populations in vivo.

### Example 5: Anti-Tumor Efficacy Induced in Human Xenograft Tumor Mouse Models by a Polypeptide Comprising a NKp46-Binding VHH and an IL-2 Variant

The in vivo anti-tumor activity of a polypeptide comprising NKp46-binding VHH domain hz5D7v17, a heterodimeric Fc region, and an attenuated IL-2 mutant fused to the C-terminus of the Fc region (cx11314) was tested in a xenograft mouse model 7-week-old female BALB-Scid mice (8 per group) were inoculated subcutaneously with 3.5×10e6 Raji cells in 100 μL HBSS. When tumor masses reached an average of 100 mm³, animals were dosed with either 1 mg/kg of cx1134, 5 mg/kg of a sequence analog of the therapeutic antibody rituximab, a combination of both regimens, or vehicle control. All treatments were dosed once a week for three consecutive weeks. Cx11314 and vehicle were dosed intravenously and the rituximab-analog was dosed intraperitoneally. Tumor volumes were determined by measuring the length and width of the subcutaneous masses three times per week. Tumor volumes were calculated using the formula V=L×W×W/2. The data were plotted and analyzed using GraphPad Prism analysis software.

As shown in FIG. 7, single agent therapy using polypeptide comprising a NKp46-binding VHH domain, a heterodimeric Fc, and an IL-2 variant fused to the C-terminus of the Fc region (cx11314) delays the growth of Raji xenografts in BALB-Scid mice compared to the vehicle control. The analog of the therapeutic antibody rituximab, which recognizes CD20 on Raji tumor cells and can redirect effector cells including NK cells to recognize and kill tumor cells, induces a significant anti-tumor response and results in tumor stasis that lasts for approximately 3 weeks before tumor volumes start to increase again in the combination treatment, the polypeptide comprising a NKp46-binding VHH domain, a heterodimeric Fc, and an IL-2 variant fused to the C-terminus of the Fc region potentiates the activity of the rituximab-analog further and results in complete and durable elimination of tumors in 7/8 animals. These data show that a polypeptide comprising NKp46-binding VHH domain (e.g., hz5D7v17), a heterodimeric Fc region, and an attenuated IL-2 variant fused to the C-terminus of the Fc region induces a functional response in vivo leading to anti-tumor activity as a single agent and potently enhancing the anti-tumor response of a therapeutic antibody like rituximab.

### Example 6: Rescue of Chemotherapy-Induced Defects in NK Cell Health and Anti-Tumor Activity by a Polypeptide Comprising a NKp46-Binding VHH and an IL-2 Variant

The activity of a polypeptide comprising NKp46-binding VHH domain hz5D7v17, a heterodimeric Fc region, and an attenuated IL-2 mutant fused to the C-terminus of the Fc region (cx11314) was further assessed in combination with the standard-of-care chemotherapy reagents dexamethasone and lenalidomide. PBMCs obtained from human donors were thawed and treated for three days with combinations of 500 nM dexamethasone, 2 μM lenalidomide, and 5 nM cx11314. All pre-treatment conditions also included 2 ng/mL IL-2 to support NK cell survival. The NK cell frequency in each treated PBMC sample was quantified by flow cytometry and normalized to a media only control. To assess the antibody-dependent cellular cytotoxicity (ADCC) capacity of NK cells from the pre-treated PBMCs, NK cells were enriched and co-cultured for 18 hours with CellTrace™ Violet-labeled MM1S (Multiple myeloma) target cells at a 10 NK cell to 1 MM1 S cell ratio and 1 nM of a Daratumumab sequence analog (Anti-hCD38-hIgG1). Pre-treatment conditions were continued through the co-culture. MM1S killing was determined via flow cytometry by quantifying the percentage of MM1S cells that stained positive with the live/dead stain Zombie Aqua and/or the apoptosis marker Apotracker green.

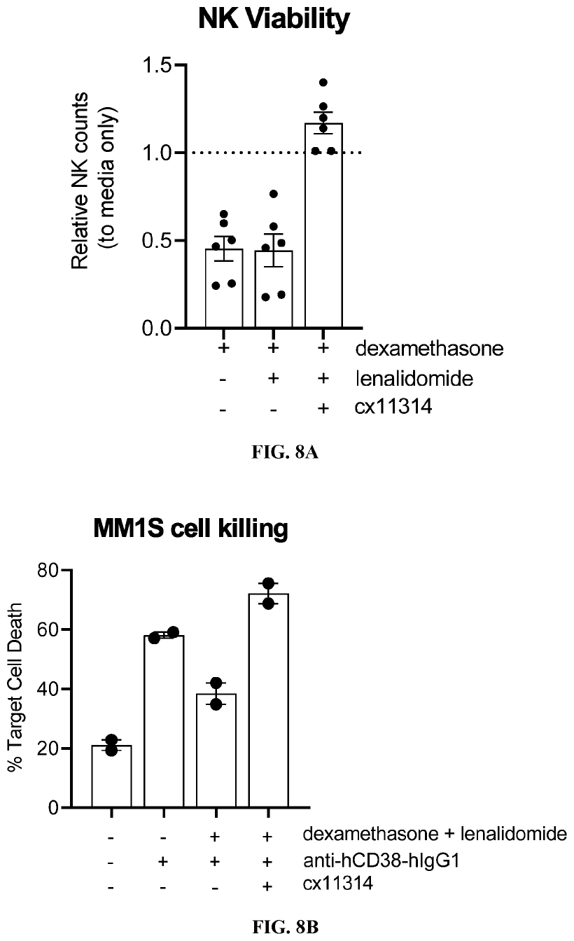

As shown in FIG. 8A, treatment of human PBMCs with standard-of-care treatments dexamethasone and lenalidomide results in an approximately 50% lower NK cell count after three days compared to a treatment with media alone. Co-treatment with cx11314 and the chemotherapy regimen recovers NK cell viability and/or proliferation and results in NK cell numbers similar to or higher than in the media control FIG. 8B shows that the ADCC activity of NK cells in the presence of a daratumumab-analog (Anti-hCD38-hIgG1) is reduced by approximately 20% when the cells were pre-treated with dexamethasone and lenalidomide. However, adding cx11314 to the pre-treatment regimen rescued or even increases the ADCC activity. These data show that a polypeptide comprising NKp46-binding VHH domain hz5D7v17, a heterodimeric Fc region, and an attenuated IL-2 mutant fused to the C-terminus of the Fe region can overcome the suppression of NK cells by standard-of-care chemotherapy treatments like dexamethasone and lenalidomide.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

Table of Certain Sequences

| SEQ | | |
|---|---|---|
| ID | | |
| NO | Description | Sequence |
| 1 | 5D7 VHH | QVTLRESGASLSLTCAASGRTFASAAMGWFRQAPGEEREFVAAISRSDDTYYA |
| | | DSVKGRFTISRDNAKNTVYLQMNSLKSEDTAVYYCAAVVPTYGNNIYVHSAAY |
| | | NYWGQGTQVTVKPG |
| 2 | hz5D7v1 VHH | EVQLVESGGGEVQPGGSLRLSCAASGRTFASAAMGWFRQAPGKGREFVAAISR |
| | | SDDTYYAESVKGRFTISRDNAKNTLYLQMSSLRAEDTAVYYCAAVVPTYGNNI |
| | | YVHSAAYNYWGQGTLVTVKP |
| 3 | hz5D7v2 VHH | EVQLVESGGGEVQPGGSLRLSCAASGRTFASAAMGWFRQAPGKEREFVAAISR |
| | | SDDTYYAESVKGRFTISRDNAKNTLYLQMSSLRAEDTAVYYCAAVVPTYGNNI |
| | | YVHSAAYNYWGQGTLVTVKP |
| 4 | hz5D7v3 VHH | EVQLVESGGGEVQPGGSLRLSCAASGRTFASAAMGWFRQAPGKEREFVAAISR |
| | | SDDTYYAESVKGRFTISRDNAKNTVYLQMSSLRAEDTAVYYCAAVVPTYGNNI |
| | | YVHSAAYNYWGQGTLVTVKP |
| 5 | hz5D7v4 VHH | EVQLVESGGGEVQPGGSLRLSCAASGRTFASAAMGWFRQAPGKEREFVAAISR |
| | | SDDTYYADSVKGRFTISRDNAKNTVYLQMSSLRAEDTAVYYCAAVVPTYGNNI |
| | | YVHSAAYNYWGQGTLVTVKP |
| 6 | hz5D7v7 VHH | EVQLVESGGGEVQPGGSLRLSCAASGRTFASAAMGWFRQAPGKEREFVAAISR |
| | | SGDTYYADSVKGRFTISRDNAKNTVYLQMSSLRAEDTAVYYCAAVVPTYGNNI |
| | | YVHSAAYNYWGQGTLVTVKP |
| 7 | hz5D7v8 VHH | EVQLVESGGGEVQPGGSLRLSCAASGRTFASAAMGWFRQAPGKEREFVAAISR |
| | | SVGDTYYADSVKGRFTISRDNAKNTVYLQMSSLRAEDTAVYYCAAVVPTYGNN |
| | | IYVHSAAYNYWGQGTLVTVKP |
| 8 | hz5D7v9 VHH | EVQLVESGGGEVQPGGSLRLSCAASGRTFASAAMGWFRQAPGKEREFVAAISR |
| | | SVDDTYYADSVKGRFTISRDNAKNTVYLQMSSLRAEDTAVYYCAAVVPTYGNN |
| | | IYVHSAAYNYWGQGTLVTVKP |
| 9 | hz5D7v10 | EVQLVESGGSLRLSCAASGRTFASAAMGWFRQAPGKEREFVAAISRSDDTYYA |
| | VHH | DSVKGRFTISRDNAKNTVYLQMSSLRAEDTAVYYCAAVVPTYGNNIYVHSAAY |
| | | NYWGQGTLVTVKP |
| 10 | hz5D7v11 | EVQLVESGGGEVQPGGSLRLSCAASGRTFASAAMGWFRQAPGKEREFVAAISR |
| | VHH | SDDTYYADSVKGRFTISRDNAKNTVYLQMSSLRAEDTAVYYCAAVVPTYGSNI |
| | | YVYSAAYNYWGQGTLVTVKP |
| 11 | hz5D7v12 | EVQLVESGGGEVQPGGSLRLSCAASGRTFASAAMGWFRQAPGKEREFVAAISR |
| | VHH | SDDTYYAESVKGRFTISRDNAKNTVYLQMSSLRAEDTAVYYCAAVVPTYGSNI |
| | | YVHSAAYNYWGQGTLVTVKP |
| 12 | hz5D7v13 | EVQLVESGGGEVQPGGSLRLSCAASGRTFASAAMGWFRQAPGKEREFVAAISR |
| | VHH | SDDTYYAESVKGRFTISRDNAKNTVYLQMSSLRAEDTAVYYCAAVVPTYGNTI |
| | | YVHSAAYNYWGQGTLVTVKP |
| 13 | hz5D7v14 | EVQLVESGGGEVQPGGSLRLSCAASGRTFASAAMGWFRQAPGKEREFVAAISR |
| | VHH | SDDTYYAESVKGRFTISRDNAKNTVYLQMSSLRAEDTAVYYCAAVVPTYGSSI |
| | | YVHSAAYNYWGQGTLVTVKP |
| 14 | hz5D7v15 | EVQLVESGGGEVQPGGSLRLSCAASGRTFASAAMGWFRQAPGKEREFVAAISR |
| | VHH | SDDTYYAESVKGRFTISRDNAKNTVYLQMSSLRAEDTAVYYCAAVVPTYGNAI |
| | | YVHSAAYNYWGQGTLVTVKP |
| 15 | hz5D7v17 | EVQLVESGGGEVQPGGSLRLSCAASGRTFASAAMGWFRQAPGKEREFVAAISR |
| | VHH | SDDTYYAESVKGRFTISRDNAKNTVYLQMSSLRAEDTAVYYCAAVVPTYGSNI |
| | | YVHSAAYNYWGQGTLVTVRP |
| 16 | hz5D7v12L | EVQLLESGGGEVQPGGSLRLSCAASGRTFASAAMGWFRQAPGKEREFVAAISR |
| | VHH | SDDTYYAESVKGRFTISRDNAKNTVYLQMSSLRAEDTAVYYCAAVVPTYGSNI |
| | | YVHSAAYNYWGQGTLVTVKP |
| 17 | CDR1 of 5D7, | GRTFASAAMG |
| | hz5D7v1, | |
| | hz5D7v2, | |
| | hz5D7v3, | |
| | hz5D7v4, | |
| | hz5D7v7, | |
| | hz5D7v8, | |
| | hz5D7v9, | |
| | hz5D7v10, | |
| | hz5D7v11, | |
| | hz5D7v12, | |
| | hz5D7v13, | |
| | hz5D7v14, | |
| | hz5D7v15, | |
| | hz5D7v12L, | |
| | and hz5D7v17 | |
| 18 | CDR2 of 5D7, | AISRSDDTY |
| | hz5D7v1, | |
| | hz5D7v2, | |
| | hz5D7v3, | |
| | hz5D7v4, | |
| | hz5D7v10, | |
| | hz5D7v11, | |
| | hz5D7v12, | |
| | hz5D7v13, | |
| | hz5D7v14, | |
| | hz5D7v15, | |
| | hz5D7v12L, | |
| | and hzD7v17 | |
| 19 | CDR2 of | AISRSGDTY |
| | hz5D7v7 | |
| 20 | CDR2 of | AISRSVGDTY |
| | hz5D7v8 | |
| 21 | CDR2 of | AISRSVDDTY |
| | hz5D7v9 | |
| 22 | CDR3 of 5D7, | VVPTYGNNIYVHSAAYNY |
| | hz5D7v1, | |
| | hz5D7v2, | |
| | hz5D7v3, | |
| | hz5D7v4, | |
| | hz5D7v7, | |
| | hz5D7v8, | |
| | hz5D7v9, and | |
| | hz5D7v10 | |
| 23 | CDR3 of | VVPTYGSNIYVYSAAYNY |
| | hz5D7v11 | |
| 24 | CDR3 of | VVPTYGSNIYVHSAAYNY |
| | hz5D7v12, | |
| | hz5D7v12L, | |
| | and hz5D7v17 | |
| 25 | CDR3 of | VVPTYGNTIYVHSAAYNY |
| | hz5D7v13 | |
| 26 | CDR3 of | VVPTYGSSIYVHSAAYNY |
| | hz5D7v14 | |
| 27 | CDR3 of | VVPTYGNAIYVHSAAYNY |
| | hz5D7v15 | |
| 29 | Human NKp46 | MSSTLPALLCVGLCLSQRISAQQQTLPKPFIWAEPHFMVPKEKQVTICCQGNY |
| | | GAVEYQLHFEGSLFAVDRPKPPERINKVKFYIPDMNSRMAGQYSCIYRVGELW |
| | | SEPSNLLDLVVTEMYDTPTLSVHPGPEVISGEKVTFYCRLDTATSMFLLLKEG |
| | | RSSHVQRGYGKVQAEFPLGPVTTAHRGTYRCFGSYNNHAWSFPSEPVKLLVTG |
| | | DIENTSLAPEDPTFPADTWGTYLLTTETGLQKDHALWDHTAQNLLRMGLAFLV |
| | | LVALVWFLVEDWLSRKRTRERASRASTWEGRRRLNTQTL |
| 30 | Wild type | APTSSSTKKTQLQLEHLLLDLQMILNGINNYKNPKLTRMLTFKFYMPKKATEL |
| | human IL-2 | KHLQCLEEELKPLEEVLNLAQSKNFHLRPRDLISNINVIVLELKGSETTFMCE |
| | | YADETATIVEFLNRWITFCQSIISTLT |
| 31 | IL-2-T3A | APASSSTKKTQLQLEALLLDLQMILNGINNYKNPKLTRMLTFKFYMPKKATEL |
| | H16A E61R | KHLQCLERELKRLEEVLNLAQSKNFHLRPRYLISNINVIVLELKGSETTFMCE |
| | P65R D84Y | YADETATIVEFLNRWITFSQSIISTLT |
| | C125S | |
| 32 | IL-2-T3A- | APASSSTKKTQLQLEALLLDLQMILNGINNYKNPKLTRMLTFKFYMPKKATEL |
| | H16A-P65R- | KHLQCLEEELKRLEEVLNLAQSKNFHLRPRSLISNINVIVLELKGSETTFMCE |
| | D84S-C125S | YADETATIVEFLNRWITFSQSIISTLT |
| 33 | hz5D7v12-Fc | EVQLVESGGGEVQPGGSLRLSCAASGRTFASAAMGWFRQAPGKEREFVAAISR |
| | xELL-Knob- | SDDTYYAESVKGRFTISRDNAKNTVYLQMSSERAEDTAVYYCAAVVPTYGSNI |
| | IL2-T3A- | YVHSAAYNYWGQGTLVTVKPGGGGDKTHTCPPCPAPGGPSVFLFPPKPKDTLM |
| | H16A-E61R- | ISRTPEVTCVVVDVSHEDPEVKFNWYVDGVEVHNAKTKPREEQYNSTYRVVSV |
| | P65R-D84Y- | LTVLHQDWLNGKEYKCKVSNKALPAPIEKTISKAKGQPREPQVYTLPPCRDEL |
| | C125S | TKNQVSLWCLVKGFYPSDIAVEWESNGQPENNYKTTPPVLDSDGSFFLYSKLT |
| | | VDKSRWQQGNVFSCSVMHEALHNHYTQKSLSLSPGGSGGSAPASSSTKKTQLQ |
| | | LEALLLDLQMILNGINNYKNPKLTRMLTFKFYMPKKATELKHLQCLERELKRL |
| | | EEVLNLAQSKNFHLRPRYLISNINVIVLELKGSETTFMCEYADETATIVEFLN |
| | | RWITFSQSIISTLT |
| 34 | hz5D7v12-Fc | EVQLVESGGGEVQPGGSLRLSCAASGRTFASAAMGWFRQAPGKEREFVAAISR |
| | xELL-Hole | SDDTYYAESVKGRFTISRDNAKNTVYLQMSSLRAEDTAVYYCAAVVPTYGSNI |
| | | YVHSAAYNYWGQGTLVTVKPGGGGDKTHTCPPCPAPGGPSVFLFPPKPKDTLM |
| | | ISRTPEVTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVCTLPPSRDEL |
| | | TKNQVSLSCAVKGFYPSDIAVEWESNGQPENNYKTTPPVLDSDGSFFLVSKLT |
| | | VDKSRWQQGNVFSCSVMHEALHNRYTQKSLSLSPG |
| 35 | hz5D7v12-Fc | EVQLVESGGGEVQPGGSLRLSCAASGRTFASAAMGWFRQAPGKEREFVAAISR |
| | xELL-Knob | SDDTYYAESVKGRFTISRDNAKNTVYLQMSSLRAEDTAVYYCAAVVPTYGSNI |
| | | YVHSAAYNYWGQGTLVTVKPGGGGDKTHTCPPCPAPGGPSVFLFPPKPKDTLM |
| | | ISRTPEVTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLPPCRDEL |
| | | TKNQVSLWCLVKGFYPSDIAVEWESNGQPENNYKTTPPVLDSDGSFFLYSKLT |
| | | VDKSRWQQGNVFSCSVMHEALHNHYTQKSLSLSPG |
| 36 | hz5D7v12-Fc | EVQLVESGGGEVQPGGSLRLSCAASGRTFASAAMGWFRQAPGKEREFVAAISR |
| | xELL-Hole | SDDTYYAESVKGRFTISRDNAKNTVYLQMSSLRAEDTAVYYCAAVVPTYGSNI |
| | | YVHSAAYNYWGQGTLVTVKPGGGGDKTHTCPPCPAPGGPSVFLFPPKPKDTLM |
| | | ISRTPEVTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVCTLPPSRDEL |
| | | TKNQVSLSCAVKGFYPSDIAVEWESNGQPENNYKTTPPVLDSDGSFFLVSKLT |
| | | VDKSRWQQGNVFSCSVMHEALHNRYTQKSLSLSPG |
| 37 | Non-targeted | EVQLVESGGGEVQPGGSLRLSCAASGSINSINVMEWYRQAPGKERDLVAGITS |
| | VHH-Fc | DGDTNYAESVKGRFTISRDNAKNTLYLQMSSLRAEDTAVYYCRARDWGSLTDY |
| | xELL-Knob- | WGQGTLVTVKPGGGGDKTHTCPPCPAPGGPSVFLFPPKPKDTLMISRTPEVTC |
| | IL2-T3A | VVVDVSHEDPEVKFNWYVDGVEVHNAKTKPREEQYNSTYRVVSVLTVLHQDWL |
| | H16A E61R | NGKEYKCKVSNKALPAPIEKTISKAKGQPREPQVYTLPPCRDELTKNQVSLWC |
| | P65R D84Y | LVKGFYPSDIAVEWESNGQPENNYKTTPPVLDSDGSFFLYSKLTVDKSRWQQG |
| | C125S | NVFSCSVMHEALHNHYTQKSLSLSPGGSGGSAPASSSTKKTQLQLEALLLDLQ |
| | | MILNGINNYKNPKLTRMLTFKFYMPKKATELKHLQCLERELKRLEEVLNLAQS |
| | | KNFHLRPRYLISNINVIVLELKGSETTFMCEYADETATIVEFLNRWITESQSI |
| | | ISTLT |
| 38 | Non-targeted | EVQLVESGGGEVQPGGSLRLSCAASGSINSINVMEWYRQAPGKERDLVAGITS |
| | VHH-Fc | DGDTNYAESVKGRFTISRDNAKNTLYLQMSSLRAEDTAVYYCPARDWGSLTDY |
| | xELL-Hole | WGQGTLVTVKPGGGGDKTHTCPPCPAPGGPSVFLFPPKPKDTLMISRTPEVTC |
| | | VVVDVSHEDPEVKFNWYVDGVEVHNAKTKPREEQYNSTYRVVSVLTVLHQDWL |
| | | NGKEYKCKVSNKALPAPIEKTISKAKGQPREPQVCTLPPSRDELTKNQVSLSC |
| | | AVKGFYPSDIAVEWESNGQPENNYKTTPPVLDSDGSFFLVSKLTVDKSRWQQG |
| | | NVFSCSVMHEALHNRYTQKSLSLSPGK |
| 40 | hz5D7v12-Fc | EVQLVESGGGEVQPGGSLRLSCAASGRTFASAAMGWFRQAPGKEREFVAAISR |
| | xELL-Hole | SDDTYYAESVKGRFTISRDNAKNTVYLQMSSLRAEDTAVYYCAAVVPTYGSNI |
| | (with C- | YVHSAAYNYWGQGTLVTVKPGGGGDKTHTCPPCPAPGGPSVFLFPPKPKDTLM |
| | terminal | ISRTPEVTCVVVDVSHEDPEVKFNWYVDGVEVHNAKTKPREEQYNSTYRVVSV |
| | lysine) | LTVLHQDWLNGKEYKCKVSNKALPAPIEKTISKAKGQPREPQVCTLPPSRDEL |
| | | TKNQVSLSCAVKGFYPSDIAVEWESNGQPENNYKTTPPVLDSDGSFFLVSKLT |
| | | VDKSRWQQGNVFSCSVMHEALHNRYTQKSLSLSPGK |
| 41 | hz5D7v17-Fc | EVQLVESGGGEVQPGGSLRLSCAASGRTFASAAMGWFRQAPGKEREFVAAISR |
| | xELL-Knob- | SDDTYYAESVKGRFTISRDNAKNTVYLQMSSLRAEDTAVYYCAAVVPTYGSNI |
| | IL2-T3A- | YVHSAAYNYWGQGTLVTVRPGGGGDKTHTCPPCPAPGGPSVFLFPPKPKDTLM |
| | H16A-E61R- | ISRTPEVTCVVVDVSHEDPEVKFNWYVDGVEVHNAKTKPREEQYNSTYRVVSV |
| | P65R-D84Y- | LTVLHQDWLNGKEYKCKVSNKALPAPIEKTISKAKGQPREPQVYTLPPCRDEL |
| | C125S | TKNQVSLWCLVKGFYPSDIAVEWESNGQPENNYKTTPPVLDSDGSFFLYSKLT |
| | | VDKSRWQQGNVFSCSVMHEALHNHYTQKSLSLSPGGSGGSAPASSSTKKTQLQ |
| | | LEALLLDLQMILNGINNYKNPKLTRMLTFKFYMPKKATELKHLQCLERELKRL |
| | | EEVLNLAQSKNFHLRPRYLISNINVIVLELKGSETTFMCEYADETATIVEFLN |
| | | RWITFSQSIISTLT |
| 42 | hz5D7v17-Fc | EVQLVESGGGEVQPGGSLRLSCAASGRTFASAAMGWFRQAPGKEREFVAAISR |
| | xELL-Hole | SDDTYYAESVKGRFTISRDNAKNTVYLQMSSLRAEDTAVYYCAAVVPTYGSNI |
| | (with C- | YVHSAAYNYWGQGTLVTVRPGGGGDKTHTCPPCPAPGGPSVFLFPPKPKDTLM |
| | terminal | ISRTPEVTCVVVDVSHEDPEVKFNWYVDGVEVHNAKTKPREEQYNSTYRVVSV |
| | lysine) | LTVLHQDWLNGKEYKCKVSNKALPAPIEKTISKAKGQPREPQVCTLPPSRDEL |
| | | TKNQVSLSCAVKGFYPSDIAVEWESNGQPENNYKTTPPVLDSDGSFFLVSKLT |
| | | VDKSRWQQGNVFSCSVMHEALHNRYTQKSLSLSPGK |
| 43 | hz5D7v17-Fc | EVQLVESGGGEVQPGGSLRLSCAASGRTFASAAMGWFRQAPGKEREFVAAISR |
| | xELL-Hole | SDDTYYAESVKGRFTISRDNAKNTVYLQMSSLRAEDTAVYYCAAVVPTYGSNI |
| | | YVHSAAYNYWGQGTLVTVRPGGGGDKTHTCPPCPAPGGPSVFLFPPKPKDTLM |
| | | ISRTPEVTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVCTLPPSRDEL |
| | | TKNQVSLSCAVKGFYPSDIAVEWESNGQPENNYKTTPPVLDSDGSFFLVSKLT |
| | | VDKSRWQQGNVFSCSVMHEALHNRYTQKSLSLSPG |
| 45 | Linker | GGGG |
| 46 | Linker | GGSGGS |
| 47 | Linker | GGSSGS |
| 53 | Fc* | GGGGPSVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIEKTISKA |
| | | KGQPREPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSLSLSP |
| | | G |
| 54 | human IgG1 | DKTHTCPPCPAPELLGGPSVFLFPPKPKDTLMISRTPEVTCVVVDVSHEDPEV |
| | Fc region | KFNWYVDGVEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSVMHEAL |
| | | HNHYTQKSLSLSPG |
| 55 | human IgG1 | DKTHTCPPCPAPGGPSVFLFPPKPKDTLMISRTPEVTCVVVDVSHEDPEVKFN |
| | xELL FC | WYVDGVEVHNAKTKPREEQYNSTYRVVSVLTVLHQDWLNGKEYKCKVSNKALP |
| | region | APIEKTISKAKGQPREPQVYTLPPSRDELTKNQVSLTCLVKGFYPSDIAVEWE |
| | | SNGQPENNYKTTPPVLDSDGSFFLYSKLTVDKSRWQQGNVFSCSVMHEALHNH |
| | | YTQKSLSLSPG |
| 56 | Fc region | DKTHTCPPCPAPELLGGPSVFLFPPKPKDTLYISRTPEVTCVVVDVSHEDPEV |
| | M252Y and | KFNWYVDGVEVHNAKTKPREEQYNSTYRVVSVLTVLHQDWLNGKEYKCKVSNK |
| | M428V (YV) | ALPAPIEKTISKAKGQPREPQVYTLPPCRDELTKNQVSLWCLVKGFYPSDIAV |
| | S354C T366W | EWESNGQPENNYKTTPPVLDSDGSFFLYSKLTVDKSRWQQGNVFSCSVVHEAL |
| | knob | HNHYTQKSLSLSPG |
| 57 | Fc region | DKTHTCPPCPAPELLGGPSVFLFPPKPKDTLYISRTPEVTCVVVDVSHEDPEV |
| | M252Y, | KFNWYVDGVEVHNAKTKPREEQYNSTYRVVSVLTVLHQDWLNGKEYKCKVSNK |
| | M428V, | ALPAPIEKTISKAKGQPREPQVCTLPPSRDELTKNQVSLSCAVKGFYPSDIAV |
| | H435R (YVR) | EWESNGQPENNYKTTPPVLDSDGSFFLVSKLTVDKSRWQQGNVFSCSVVHEAL |
| | T366S, | HNRYTQKSLSLSPG |
| | L368A, | |
| | Y407V hole | |
| 58 | Fc region | DKTHTCPPCPAPGGPSVFLFPPKPKDTLMISRTPEVTCVVVDVSHEDPEVKFN |
| | xELL H435R | WYVDGVEVHNAKTKPREEQYNSTYRVVSVLTVLHQDWLNGKEYKCKVSNKALP |
| | | APIEKTISKAKGQPREPQVYTLPPSRDELTKNQVSLTCLVKGFYPSDIAVEWE |
| | | SNGQPENNYKTTPPVLDSDGSFFLYSKLTVDKSRWQQGNVFSCSVMHEALHNR |
| | | YTQKSLSLSPG |
| 59 | Fc region | DKTHTCPPCPAPGGPSVFLFPPKPKDTLYISRTPEVTCVVVDVSHEDPEVKFN |
| | xELL M252Y | WYVDGVEVHNAKTKPREEQYNSTYRVVSVLTVLHQDWLNGKEYKCKVSNKALP |
| | and M428V | APIEKTISKAKGQPREPQVYTLPPSRDELTKNQVSLTCLVKGFYPSDIAVEWE |
| | (YV) | SNGQPENNYKTTPPVLDSDGSFFLYSKLTVDKSRWQQGNVFSCSVVHEALHNH |
| | | YTQKSLSLSPG |
| 60 | Fc region | DKTHTCPPCPAPGGPSVFLFPPKPKDTLYISRTPEVTCVVVDVSHEDPEVKFN |
| | xELL M252Y | WYVDGVEVHNAKTKPREEQYNSTYRVVSVLTVLHQDWLNGKEYKCKVSNKALP |
| | and M428L | APIEKTISKAKGQPREPQVYTLPPSRDELTKNQVSLTCLVKGFYPSDIAVEWE |
| | (YL) | SNGQPENNYKTTPPVLDSDGSFFLYSKLTVDKSRWQQGNVFSCSVLHEALHNH |
| | | YTQKSLSLSPG |
| 61 | Fc region | DKTHTCPPCPAPGGPSVFLFPPKPKDTLYISRTPEVTCVVVDVSHEDPEVKFN |
| | xELL M252Y, | WYVDGVEVHNAKTKPREEQYNSTYRVVSVLTVLHQDWLNGKEYKCKVSNKALP |
| | M428L, | APIEKTISKAKGQPREPQVYTLPPSRDELTKNQVSLTCLVKGFYPSDIAVEWE |
| | H435R (YLR) | SNGQPENNYKTTPPVLDSDGSFFLYSKLTVDKSRWQQGNVFSCSVLHEALHNR |
| | | YTQKSLSLSPG |
| 62 | Fc region | DKTHTCPPCPAPGGPSVFLFPPKPKDTLYISRTPEVTCVVVDVSHEDPEVKFN |
| | xELL M252Y, | WYVDGVEVHNAKTKPREEQYNSTYRVVSVLTVLHQDWLNGKEYKCKVSNKALP |
| | M428V, | APIEKTISKAKGQPREPQVYTLPPSRDELTKNQVSLTCLVKGFYPSDIAVEWE |
| | H435R (YVR) | SNGQPENNYKTTPPVLDSDGSFFLYSKLTVDKSRWQQGNVFSCSVVHEALHNR |
| | | YTQKSLSLSPG |
| 63 | Fc region | DKTHTCPPCPAPGGPSVFLFPPKPKDTLMISRTPEVTCVVVDVSHEDPEVKFN |
| | xELL S354C | WYVDGVEVHNAKTKPREEQYNSTYRVVSVLTVLHQDWLNGKEYKCKVSNKALP |
| | T366W knob | APIEKTISKAKGQPREPQVYTLPPCRDELTKNQVSLWCLVKGFYPSDIAVEWE |
| | | SNGQPENNYKTTPPVLDSDGSFFLYSKLTVDKSRWQQGNVFSCSVMHEALANH |
| | | YTQKSLSLSPG |
| 64 | Fc region | DKTHTCPPCPAPGGPSVFLFPPKPKDTLMISRTPEVTCVVVDVSHEDPEVKFN |
| | xELL H435R | WYVDGVEVHNAKTKPREEQYNSTYRVVSVLTVLHQDWLNGKEYKCKVSNKALP |
| | S354C T366W | APIEKTISKAKGQPREPQVYTLPPCRDELTKNQVSLWCLVKGFYPSDIAVEWE |
| | knob | SNGQPENNYKTTPPVLDSDGSFFLYSKLTVDKSRWQQGNVFSCSVMHEALHNR |
| | | YTQKSLSLSPG |
| 65 | Fc region | DKTHTCPPCPAPGGPSVFLFPPKPKDTLYISRTPEVTCVVVDVSHEDPEVKFN |
| | xELL M252Y | WYVDGVEVHNAKTKPREEQYNSTYRVVSVLTVLHQDWLNGKEYKCKVSNKALP |
| | and M428V | APIEKTISKAKGQPREPQVYTLPPCRDELTKNQVSLWCLVKGFYPSDIAVEWE |
| | (YV) S354C | SNGQPENNYKTTPPVLDSDGSFFLYSKLTVDKSRWQQGNVFSCSVVHEALHNH |
| | T366W knob | YTQKSLSLSPG |
| 66 | Fc region | DKTHTCPPCPAPGGPSVFLFPPKPKDTLYISRTPEVTCVVVDVSHEDPEVKFN |
| | xELL M252Y | WYVDGVEVHNAKTKPREEQYNSTYRVVSVLTVLHQDWLNGKEYKCKVSNKALP |
| | and M428L | APIEKTISKAKGQPREPQVYTLPPCRDELTKNQVSLWCLVKGFYPSDIAVEWE |
| | (YL) S354C | SNGQPENNYKTTPPVLDSDGSFFLYSKLTVDKSRWQQGNVFSCSVLHEALHNH |
| | T366W knob | YTQKSLSLSPG |
| 67 | Fc region | DKTHTCPPCPAPGGPSVFLFPPKPKDTLYISRTPEVTCVVVDVSHEDPEVKFN |
| | xELL M252Y, | WYVDGVEVHNAKTKPREEQYNSTYRVVSVLTVLHQDWLNGKEYKCKVSNKALP |
| | M428L, | APIEKTISKAKGQPREPQVYTLPPCRDELTKNQVSLWCLVKGFYPSDIAVEWE |
| | H435R (YLR) | SNGQPENNYKTTPPVLDSDGSFFLYSKLTVDKSRWQQGNVFSCSVLHEALHNR |
| | S354C T366W | YTQKSLSLSPG |
| | knob | |
| 68 | Fc region | DKTHTCPPCPAPGGPSVFLFPPKPKDTLYISRTPEVTCVVVDVSHEDPEVKFN |
| | xELL M252Y, | WYVDGVEVHNAKTKPREEQYNSTYRVVSVLTVLHQDWLNGKEYKCKVSNKALP |
| | M428V, | APIEKTISKAKGQPREPQVYTLPPCRDELTKNQVSLWCLVKGFYPSDIAVEWE |
| | H435R (YVR) | SNGQPENNYKTTPPVLDSDGSFFLYSKLTVDKSRWQQGNVFSCSVVHEALHNR |
| | S354C T366W | YTQKSLSLSPG |
| | knob | |
| 69 | Fc region | DKTHTCPPCPAPGGPSVFLFPPKPKDTLMISRTPEVTCVVVDVSHEDPEVKFN |
| | xELL T366S, | WYVDGVEVHNAKTKPREEQYNSTYRVVSVLTVLHQDWLNGKEYKCKVSNKALP |
| | L368A, | APIEKTISKAKGQPREPQVCTLPPSRDELTKNQVSLSCAVKGFYPSDIAVEWE |
| | Y407V hole | SNGQPENNYKTTPPVLDSDGSFFLVSKLTVDKSRWQQGNVFSCSVMHEALHNH |
| | | YTQKSISLSPG |
| 70 | Fc region | DKTHTCPPCPAPGGPSVFLFPPKPKDTIMISRTPEVTCVVVDVSHEDPEVKFN |
| | xELL H435R, | WYVDGVEVHNAKTKPREEQYNSTYRVVSVLTVLHQDWLNGKEYKCKVSNKALP |
| | T366S, | APIEKTISKAKGQPREPQVCTLPPSRDELTKNQVSLSCAVKGFYPSDIAVEWE |
| | L368A, | SNGQPENNYKTTPPVLDSDGSFFLVSKLTVDKSRWQQGNVFSCSVMHEALHNR |
| | Y407V hole | YTQKSLSLSPG |
| 44 | Fc region | DKTHTCPPCPAPGGPSVFLFPPKPKDTLMISRTPEVTCVVVDVSHEDPEVKFN |
| | xELL H435R, | WYVDGVEVENAKTKPREEQYNSTYRVVSVLTVLHQDWLNGKEYKCKVSNKALP |
| | T366S, | APIEKTISKAKGQPREPQVCTLPPSRDELTKNQVSLSCAVKGFYPSDIAVEWE |
| | L368A, | SNGQPENNYKTTPPVLDSDGSFFLVSKLTVDKSRWQQGNVFSCSVMHEALHNR |
| | Y407V hole | YTQKSLSLSPGK |
| | (with C- | |
| | terminal | |
| | lysine) | |
| 71 | Fc region | DKTHTCPPCPAPGGPSVFLFPPKPKDTLYISRTPEVTCVVVDVSHEDPEVKFN |
| | xELL M252Y | WYVDGVEVHNAKTKPREEQYNSTYRVVSVLTVLHQDWLNGKEYKCKVSNKALP |
| | and M428V | APIEKTISKAKGQPREPQVCTLPPSRDELTKNQVSLSCAVKGFYPSDIAVEWE |
| | (YV) T366S, | SNGQPENNYKTTPPVLDSDGSFFLVSKLTVDKSRWQQGNVFSCSVVHEALHNH |
| | L368A, | YTQKSLSLSPG |
| | Y407V hole | |
| 72 | Fc region | DKTHTCPPCPAPGGPSVFLFPPKPKDTLYISRTPEVTCVVVDVSHEDPEVKFN |
| | xELL M252Y | WYVDGVEVHNAKTKPREEQYNSTYRVVSVLTVLHQDWLNGKEYKCKVSNKALP |
| | and M428L | APIEKTISKAKGQPREPQVCTLPPSRDELTKNQVSLSCAVKGFYPSDIAVEWE |
| | (YL) T366S, | SNGQPENNYKTTPPVLDSDGSFFLVSKLTVDKSRWQQGNVFSCSVLHEALHNH |
| | L368A, | YTQKSLSLSPG |
| | Y407V hole | |
| 73 | Fc region | DKTHTCPPCPAPGGPSVFLFPPKPKDTLYISRTPEVTCVVVDVSHEDPEVKFN |
| | xELL M252Y, | WYVDGVEVHNAKTKPREEQYNSTYRVVSVLTVLHQDWLNGKEYKCKVSNKALP |
| | M428L, | APIEKTISKAKGQPREPQVCTLPPSRDELTKNQVSLSCAVKGFYPSDIAVEWE |
| | H435R (YLR) | SNGQPENNYKTTPPVLDSDGSFFLVSKLTVDKSRWQQGNVFSCSVLHEALHNR |
| | T366S, | YTQKSLSLSPG |
| | L368A, | |
| | Y407V hole | |
| 74 | Fc region | DKTHTCPPCPAPGGPSVFLFPPKPKDTLYISRTPEVTCVVVDVSHEDPEVKFN |
| | xELL M252Y, | WYVDGVEVHNAKTKPREEQYNSTYRVVSVLTVLHQDWLNGKEYKCKVSNKALP |
| | M428V, | APIEKTISKAKGQPREPQVCTLPPSRDELTKNQVSLSCAVKGFYPSDIAVEWE |
| | H435R (YVR) | SNGQPENNYKTTPPVLDSDGSFFLVSKLTVDKSRWQQGNVFSCSVVHEALHNR |
| | T366S, | YTQKSLSLSPG |
| | L368A, | |
| | Y407V hole | |
| 75 | Fc region | DKTHTCPPCPAPELLGGPSVFLFPPKPKDTLMISRTPEVTCVVVDVSHEDPEV |
| | H435R | KFNWYVDGVEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSVMHEAL |
| | | HNRYTQKSLSLSPG |
| 76 | Fc region | DKTHTCPPCPAPELLGGPSVFLFPPKPKDTLYISPTPEVTCVVVDVSHEDPEV |
| | M252Y and | KFNWYVDGVEVHNAKTKPREEQYNSTYRVVSVLTVLHQDWLNGKEYKCKVSNK |
| | M428V (YV) | ALPAPIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSVVHEAL |
| | | HNHYTQKSLSLSPG |
| 77 | Fc region | DKTHTCPPCPAPELLGGPSVFLFPPKPKDTLYISRTPEVTCVVVDVSHEDPEV |
| | M252Y and | KFNWYVDGVEVHNAKTKPREEQYNSTYRVVSVLTVLHQDWLNGKEYKCKVSNK |
| | M428L (YL) | ALPAPIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSVLHEAL |
| | | HNHYTQKSLSLSPG |
| 78 | Fc region | DKTHTCPPCPAPELLGGPSVFLFPPKPKDTLYISPTPEVTCVVVDVSHEDPEV |
| | M252Y, | KFNWYVDGVEVHNAKTKPREEQYNSTYRVVSVLTVLHQDWLNGKEYKCKVSNK |
| | M428L, | ALPAPIEKTISKAKGQPREPQVYTLPPSRDELTKNQVSLTCLVKGFYPSDIAV |
| | H435R (YLR) | EWESNGQPENNYKTTPPVLDSDGSFFLYSKLTVDKSRWQQGNVFSCSVLHEAL |
| | | HNRYTQKSLSLSPG |
| 79 | Fc region | DKTHTCPPCPAPELLGGPSVFLFPPKPKDTLYISRTPEVTCVVVDVSHEDPEV |
| | M252Y, | KFNWYVDGVEVHNAKTKPREEQYNSTYRVVSVLTVLHQDWLNGKEYKCKVSNK |
| | M428V, | ALPAPIEKTISKAKGQPREPQVYTLPPSRDELTKNQVSLTCLVKGFYPSDIAV |
| | H435R (YVR) | EWESNGQPENNYKTTPPVLDSDGSFFLYSKLTVDKSRWQQGNVFSCSVVHEAL |
| | | HNRYTQKSLSLSPG |
| 80 | Fc region | DKTHTCPPCPAPELLGGPSVFLFPPKPKDTLMISRTPEVTCVVVDVSHEDPEV |
| | S354C T366W | KFNWYVDGVEVHNAKTKPREEQYNSTYRVVSVLTVLHQDWLNGKEYKCKVSNK |
| | knob | ALPAPIEKTISKAKGQPREPQVYTLPPCRDELTKNQVSLWCLVKGFYPSDIAV |
| | | EWESNGQPENNYKTTPPVLDSDGSFFLYSKLTVDKSRWQQGNVFSCSVMHEAL |
| | | HNHYTQKSLSLSPG |
| 81 | Fc region | DKTHTCPPCPAPELLGGPSVFLFPPKPKDTLMISRTPEVTCVVVDVSHEDPEV |
| | H435R S354C | KFNWYVDGVEVHNAKTKPREEQYNSTYRVVSVLTVLHQDWLNGKEYKCKVSNK |
| | T366W knob | ALPAPIEKTISKAKGQPREPQVYTLPPCRDELTKNQVSLWCLVKGFYPSDIAV |
| | | EWESNGQPENNYKTTPPVLDSDGSFFLYSKLTVDKSRWQQGNVFSCSVMHEAL |
| | | HNRYTQKSLSLSPG |
| 82 | Fc region | DKTHTCPPCPAPELLGGPSVFLFPPKPKDTLYISPTPEVTCVVVDVSHEDPEV |
| | M252Y and | KFNWYVDGVEVHNAKTKPREEQYNSTYRVVSVLTVLHQDWLNGKEYKCKVSNK |
| | M428L (YL) | ALPAPIEKTISKAKGQPREPQVYTLPPCRDELTKNQVSLWCLVKGFYPSDIAV |
| | S354C T366W | EWESNGQPENNYKTTPPVLDSDGSFFLYSKLTVDKSRWQQGNVFSCSVLHEAL |
| | knob | HNHYTQKSLSLSPG |
| 83 | Fc region | DKTHTCPPCPAPELLGGPSVFLFPPKPKDTLYISRTPEVTCVVVDVSHEDPEV |
| | M252Y, | KFNWYVDGVEVHNAKTKPREEQYNSTYRVVSVLTVLHQDWLNGKEYKCKVSNK |
| | M428L, | ALPAPIEKTISKAKGQPREPQVYTLPPCRDELTKNQVSLWCLVKGFYPSDIAV |
| | H435R (YLR) | EWESNGQPENNYKTTPPVLDSDGSFFLYSKLTVDKSRWQQGNVFSCSVLHEAL |
| | S354C T366W | HNRYTQKSLSLSPG |
| | knob | |
| 84 | Fc region | DKTHTCPPCPAPELLGGPSVFLFPPKPKDTLYISRTPEVTCVVVDVSHEDPEV |
| | M252Y, | KFNWYVDGVEVHNAKTKPREEQYNSTYRVVSVLTVLHQDWLNGKEYKCKVSNK |
| | M428V, | ALPAPIEKTISKAKGQPREPQVYTLPPCRDELTKNQVSLWCLVKGFYPSDIAV |
| | H435R (YVR) | EWESNGQPENNYKTTPPVLDSDGSFFLYSKLTVDKSRWQQGNVFSCSVVHEAL |
| | S354C T366W | HNRYTQKSLSLSPG |
| | knob | |
| 85 | Fc region | DKTHTCPPCPAPELLGGPSVFLFPPKPKDTLMISRTPEVTCVVVDVSHEDPEV |
| | T366S, | KFNWYVDGVEVHNAKTKPREEQYNSTYRVVSVLTVLHQDWLNGKEYKCKVSNK |
| | L368A, | ALPAPIEKTISKAKGQPREPQVCTLPPSRDELTKNQVSLSCAVKGFYPSDIAV |
| | Y407V hole | EWESNGQPENNYKTTPPVLDSDGSFFLVSKLTVDKSRWQQGNVFSCSVMHEAL |
| | | HNHYTQKSLSLSPG |
| 86 | Fc region | DKTHTCPPCPAPELLGGPSVFLFPPKPKDTLMISRTPEVTCVVVDVSHEDPEV |
| | H435R, | KFNWYVDGVEVHNAKTKPRERQYNSTYRVVSVLTVLHQDWLNGKEYKCKVSNK |
| | T366S, | ALPAPIEKTISKAKGQPREPQVCTLPPSRDELTKNQVSLSCAVKGFYPSDIAV |
| | L368A, | EWESNGQPENNYKTTPPVLDSDGSFFLVSKLTVDKSRWQQGNVFSCSVMHEAL |
| | Y407V hole | HNRYTQKSLSLSPG |
| 87 | Fc region | DKTHTCPPCPAPELLGGPSVFLFPPKPKDTLYISRTPEVTCVVVDVSHEDPEV |
| | M252Y and | KFNWYVDGVEVHNAKTKPREEQYNSTYRVVSVLTVLHQDWLNGKEYKCKVSNK |
| | M428V (YV) | ALPAPIEKTISKAKGQPREPQVCTLPPSRDELTKNQVSLSCAVKGFYPSDIAV |
| | T366S, | EWESNGQPENNYKTTPPVLDSDGSFFLVSKLTVDKSRWQQGNVFSCSVVHEAL |
| | L368A, | HNHYTQKSLSLSPG |
| | Y407V hole | |
| 88 | Fc region | DKTHTCPPCPAPELLGGPSVFLFPPKPKDTLYISRTPEVTCVVVDVSHEDPEV |
| | M252Y and | KFNWYVDGVEVHNAKTKPREEQYNSTYRVVSVLTVLHQDWLNGKEYKCKVSNK |
| | M428L (YL) | ALPAPIEKTISKAKGQPREPQVCTLPPSRDELTKNQVSLSCAVKGFYPSDIAV |
| | T366S, | EWESNGQPENNYKTTPPVLDSDGSFFLVSKLTVDKSRWQQGNVFSCSVLHEAL |
| | L368A, | HNHYTQKSLSLSPG |
| | Y407V hole | |
| 89 | Fc region | DKTHTCPPCPAPELLGGPSVFLFPPKPKDTLYISRTPEVTCVVVDVSHEDPEV |
| | M252Y, | KFNWYVDGVEVHNAKTKPREEQYNSTYRVVSVLTVLHQDWLNGKEYKCKVSNK |
| | M428L, | ALPAPIEKTISKAKGQPREPQVCTLPPSRDELTKNQVSLSCAVKGFYPSDIAV |
| | H435R (YLR) | EWESNGQPENNYKTTPPVLDSDGSFFLVSKLTVDKSRWQQGNVFSCSVLHEAL |
| | T366S, | HNRYTQKSLSLSPG |
| | L368A, | |
| | Y407V hole | |

What is claimed is:

1. A polypeptide comprising at least one VHH domain that binds NKp46 and a modified IL-2, wherein at least one VHH domain that binds NKp46 comprises a CDR1 comprising the amino acid sequence of SEQ ID NO: 17; a CDR2 comprising the amino acid sequence of SEQ ID NO: 18, 19, 20, or 21; and a CDR3 comprising the amino acid sequence of SEQ ID NO: 22, 23, 24, 25, 26, or 27, and wherein the modified IL-2 comprises T3A, H16A, P65R, C125S mutations and a D84S or a D84Y mutation relative to a wild type human IL-2 comprising the amino acid sequence of SEQ ID NO: 30.

2. The polypeptide of claim 1, wherein the modified IL-2 comprises T3A, H16A, P65R, C125S, and D84S mutations.

3. The polypeptide of claim 1, wherein the modified IL-2 comprises T3A, H16A, E61R, P65R, C125S, and D84Y mutations.

4. The polypeptide of claim 1 or 2, wherein the modified IL-2 comprises the amino acid sequence of SEQ ID NO: 31.

5. The polypeptide of claim 1 or 3, wherein the modified IL-2 comprises the amino acid sequence of SEQ ID NO: 32.

6. The polypeptide of any one of claims 1-5, wherein at least one VHH domain comprises a CDR1 comprising the amino acid sequence of SEQ ID NO: 17; a CDR2 comprising the amino acid sequence of SEQ ID NO: 18; and a CDR3 comprising the amino acid sequence of SEQ ID NO: 22.

7. The polypeptide of any one of claims 1-5, wherein at least one VHH domain comprises a CDR1 comprising the amino acid sequence of SEQ ID NO: 17; a CDR2 comprising the amino acid sequence of SEQ ID NO: 19; and a CDR3 comprising the amino acid sequence of SEQ ID NO: 22.

8. The polypeptide of any one of claims 1-5, wherein at least one VHH domain comprises a CDR1 comprising the amino acid sequence of SEQ ID NO: 17; a CDR2 comprising the amino acid sequence of SEQ ID NO: 20; and a CDR3 comprising the amino acid sequence of SEQ ID NO: 22.

9. The polypeptide of any one of claims 1-5, wherein at least one VHH domain comprises a CDR1 comprising the amino acid sequence of SEQ ID NO: 17; a CDR2 comprising the amino acid sequence of SEQ ID NO: 21; and a CDR3 comprising the amino acid sequence of SEQ ID NO: 22.

10. The polypeptide of any one of claims 1-5, wherein at least one VHH domain comprises a CDR1 comprising the amino acid sequence of SEQ ID NO: 17; a CDR2 comprising the amino acid sequence of SEQ ID NO: 18; and a CDR3 comprising the amino acid sequence of SEQ ID NO: 23.

11. The polypeptide of any one of claims 1-5, wherein at least one VHH domain comprises a CDR1 comprising the amino acid sequence of SEQ ID NO: 17; a CDR2 comprising the amino acid sequence of SEQ ID NO: 18; and a CDR3 comprising the amino acid sequence of SEQ ID NO: 24.

12. The polypeptide of any one of claims 1-5, wherein at least one VHH domain comprises a CDR1 comprising the amino acid sequence of SEQ ID NO: 17; a CDR2 comprising the amino acid sequence of SEQ ID NO: 18; and a CDR3 comprising the amino acid sequence of SEQ ID NO: 25.

13. The polypeptide of any one of claims 1-5, wherein at least one VHH domain comprises a CDR1 comprising the amino acid sequence of SEQ ID NO: 17; a CDR2 comprising the amino acid sequence of SEQ ID NO: 18; and a CDR3 comprising the amino acid sequence of SEQ ID NO: 26.

14. The polypeptide of any one of claims 1-5, wherein at least one VHH domain comprises a CDR1 comprising the amino acid sequence of SEQ ID NO: 17; a CDR2 comprising the amino acid sequence of SEQ ID NO: 18; and a CDR3 comprising the amino acid sequence of SEQ ID NO: 27.

15. The polypeptide of any one of claims 1-14, wherein at least one VHH domain is humanized.

16. The polypeptide of any one of claims 1-15, wherein at least one VHH domain comprises an amino acid sequence at least 85%, 90%, 95%, or at least 99% identical to the amino acid sequence of any one of SEQ ID NOs: 1-16.

17. The polypeptide of any one of claims 1-15, wherein at least one VHH domain comprises the amino acid sequence of any one of SEQ ID NOs: 1-16.

18. The polypeptide of any one of claims 1-17, comprising two VHH domains.

19. The polypeptide of any one of claims 1-17, comprising three VHH domains.

20. The polypeptide of any one of claims 1-19, wherein the polypeptide comprises at least one binding domain that binds an antigen other than NKp46.

21. The polypeptide of any one of claims 1-20, wherein the polypeptide comprises at least one binding domain that binds a tumor antigen.

22. The polypeptide of any one of claims 1-21, wherein the polypeptide comprises at least one binding domain that binds an antigen selected from 1-92-LFA-3, 5T4, Alpha-4 integrin, Alpha-V integrin, alpha4beta1 integrin, alpha4beta7 integrin, AGR2, Anti-Lewis-Y, Apelin J receptor, APRIL, B7-H3, B7-H4, B7-H6, BAFF, BCMA, BTLA, C5 complement, C-242, CA9, CA19-9, (Lewis a), Carbonic anhydrase 9, CD2, CD3, CD6, CD9, CD11a, CD19, CD20, CD22, CD24, CD25, CD27, CD28, CD30, CD33, CD38, CD39, CD40, CD40L, CD41, CD44, CD44v6, CD47, CD51, CD52, CD56, CD64, CD70, CD71, CD73, CD74, CD80, CD81, CD86, CD95, CD117, CD123, CD125, CD132, (IL-2RG), CD133, CD137, CD138, CD166, CD172A, CD248, CDH6, CEACAM5 (CEA), CEACAM6 (NCA-90), CLAUDIN-3, CLAUDIN-4, cMet, Collagen, Cripto, CSFR, CSFR-1, CTLA-4, CTGF, CXCL10, CXCL13, CXCR1, CXCR2, CXCR4, CYR61, DL44, DLK1, DLL3, DLL4, DPP-4, DSG1, EDA, EDB, EGFR, EGFRviii, Endothelin B receptor (ETBR), ENPP3, EpCAM, EPHA2, EPHB2, ERBB3, F protein of RSV, FAP, FAS, FcRH5, FGF-2, FGF8, FGFR1, FGFR2, FGFR3, FGFR4, FLT-3, Folate receptor alpha (FRα), GAL3ST1, G-CSF, G-CSFR, GD2, GITR, GLUT1, GLUT4, GM-CSF, GM-CSFR, GP IIb/IIIa receptors, Gp130, GPIIB/IIIA, GPNMB, GPRC5D, GRP78, HAVCAR1, HER2/neu, HER3, HER4, HGF, hGH, HVEM, Hyaluronidase, ICOS, IFNalpha, IFNbeta, 1FNgamma, IgE, IgE Receptor (FceRI), IGF, IGF1R, IL1B, IL1R, IL2, IL11, IL12, IL12p40, IL-12R, IL-12Rbeta1, IL13, IL13R, IL15, IL17, IL18, IL21, IL23, IL23R, IL27/IL27R (wsx1), IL29, IL-31R, IL31/IL31R, IL2R, IL4, IL4R, IL6, IL6R, Insulin Receptor, Jagged Ligands, Jagged 1, Jagged 2, KISS1-R, LAG-3, LIF-R, Lewis X, LIGHT, LRP4, LRRC26, Ly6G6D, LyPD1, MCSP, Mesothelin, MICA, M4ICB, MRP4, MUC1, Mucin-16 (MUC16, CA-125), Na/K ATPase, NGF, Nicastrin, NKG2A, Notch Receptors, Notch 1, Notch 2, Notch 3, Notch 4, NOV, OSM-R, OX-40, PAR2, PDGF-AA, PDGF-BB, PDGFRalpha, PDGFRbeta, PD-1, PD-L1, PD-L2, Phosphatidyl-serine, P1GF, PSCA, PSMA, PSGR, RAAG12, RAGE, SLC44A4, Sphingosine 1 Phosphate, STEAP1, STEAP2, TAG-72, TAPA1, TEM-8, TGFbeta, TGFbeta receptor 1 (TGFBR1), TGFbeta receptor 2 (TGFBR2), TIGIT, TIM-3, TLR2, TLR4, TLR6, TLR7, TLR8, TLR9, TMEM31, TNFalpha, TNFR, TNFRS12A, TRAIL-R1, TRAIL-R2, Transferrin, Transferrin receptor, TRK-A, TRK-B, TROP-2 uPAR, VAP1, VCAM-1, VEGF, VEGF-A, VEGF-B, VEGF-C, VEGF-D, VEGFR1, VEGFR2, VEGFR3, VISTA, WISP-1, WISP-2, and WISP-3.

23. The polypeptide of any one of claims 1-19, wherein each VHH domain binds NKp46.

24. The polypeptide of claim 23, wherein each VHH domain comprises the same CDR1, CDR2, and CDR3 amino acid sequences.

25. The polypeptide of claim 24, wherein each VHH domain comprises the same VHH sequence.

26. The polypeptide of any one of claims 1-17, comprising one VHH domain.

27. The polypeptide of any one of claims 1-26, wherein the NKp46 is human NKp46.

28. The polypeptide of claim 27, wherein the human NKp46 comprises the sequence of SEQ ID NO: 29.

29. The polypeptide of any one of claims 1-28, wherein the polypeptide comprises an Fc region.

30. The polypeptide of claim 29, wherein the Fc region comprises an amino acid sequence selected from SEQ ID NOs: 44 and 53-89.

31. The polypeptide of claim 29 or claim 30, which forms a dimer under physiological conditions.

32. The polypeptide of any one of claims 29-31, wherein the modified IL-2 is fused to the C-terminus of the Fc region.

33. The polypeptide of any one of claims 29-32, wherein the polypeptide comprises one VHH domain that binds NKp46, an Fc region, and a modified IL-2.

34. The polypeptide of claim 33, wherein the polypeptide comprises the amino acid sequence of SEQ ID NO: 33 or 41.

35. A complex comprising a first polypeptide and a second polypeptide, wherein the first polypeptide is the polypeptide of any one of claims 1-34, wherein the first polypeptide comprises a first Fc region, and wherein the second polypeptide comprises at least one VHH domain and a second Fc region, wherein the first and second Fc regions are the same or different.

36. A complex comprising a first polypeptide and a second polypeptide, wherein the first polypeptide comprises at least one VHH domain that binds NKp46 and a first Fc region and the second polypeptide comprises a second Fc region and a modified IL-2, wherein at least one VHH domain that binds NKp46 comprises a CDR1 comprising the amino acid sequence of SEQ ID NO: 17; a CDR2 comprising the amino acid sequence of SEQ ID NO: 18, 19, 20, or 21; and a CDR3 comprising the amino acid sequence of SEQ ID NO: 22, 23, 24, 25, 26, or 27; and wherein the modified IL-2 comprises T3A or T3G, H16A, P65R, C125S, and D84S or D84Y mutations relative to a wild type human IL-2 comprising the amino acid sequence of SEQ ID NO: 30.

37. The complex of claim 36, wherein the modified IL-2 comprises T3A, H16A, P65R, C25S, and D84S mutations.

38. The complex of claim 36, wherein the modified IL-2 comprises T3A, H16A, E61R, P65R, C125S, and D84Y mutations.

39. The complex of any one of claims 36-38, wherein the modified IL-2 comprises an amino acid sequence that is at least 90%, at least 95%, at least 97%, at least 98%, at least 99%, or 100% identical to the amino acid sequence of SEQ ID NO: 31 or 32.

40. The complex of any one of claims 36, 38, and 39, wherein the modified IL-2 comprises the amino acid sequence of SEQ ID NO: 31.

41. The complex of any one of claims 36-40, wherein at least one VHH domain, or each VHH domain, is humanized.

42. The complex of any one of claim 36-41, wherein at least one VHH domain comprises an amino acid sequence at least 85%, at least 90%, at least 95%, or at least 99% identical to an amino acid sequence selected from SEQ ID NOs 1-16.

43. The complex of any one of claims 36-42, wherein at least one VHH domain comprises an amino acid sequence selected from SEQ ID NOs: 1-16.

44. The complex of any one of claims 35-43, wherein the second polypeptide comprises at least one antigen-binding domain.

45. The complex of claim 44, wherein at least one antigen-binding domain of the second polypeptide is a VHH domain.

46. The complex of claim 45, wherein the second polypeptide comprises at least one VHH domain that binds NKp46.

47. The complex of any one of claims 35-46, wherein the second polypeptide comprises at least one VHH domain that binds NKp46.

48. The complex of claim 47, wherein the second polypeptide comprises at least one VHH domain that binds NKp46 and comprises a CDR1 comprising the amino acid sequence of SEQ ID NO: 17; a CDR2 comprising the amino acid sequence of SEQ ID NO: 18, 19, 20, or 21; and a (CDR3 comprising the amino acid sequence of SEQ ID NO: 22, 23, 24, 25, 26, or 27.

49. The complex of claim 47 or 48, wherein the second polypeptide comprises at least one VHH domain that binds NKp46 and comprises a CDR1, a CDR2, and a CDR3, respectively comprising the amino acid sequences of SEQ ID NOs: 17, 18, and 22; 17, 19, and 22; 17, 20, and 22; 17, 21, and 22; 17, 18, and 23; 17, 18, and 24; 17, 18, and 25; 17, 18, and 26; or 17, 18, and 27.

50. The complex of any one of claims 47-49, wherein the second polypeptide comprises at least one VHH domain that binds NKp46 and comprises a CDR1 comprising the amino acid sequence of SEQ ID NO: 17; a CDR2 comprising the amino acid sequence of SEQ ID NO: 18; and a CDR3 comprising the amino acid sequence of SEQ ID NO: 24.

51. The complex of any one of claims 47-50, wherein at least one VHH domain of the second polypeptide binds NKp46 and comprises an amino acid sequence at least 85%, 90%, 95%, or at least 99% identical to the amino acid sequence of SEQ ID NO: 1, 2, 3, 4, 5, 6, 7, 8, 9, 10, 11, 12, 13, 14, 15, or 16.

52. The complex of any one of claims 47-51, wherein at least one VHH domain of the second polypeptide binds NKp46 and comprises the amino acid sequence of SEQ ID NO: 1, 2, 3, 4, 5, 6, 7, 8, 9, 10, 11, 12, 13, 14, 15, or 16.

53. The complex of any one of claims 47-52, wherein at least one VHH domain of the second polypeptide binds NKp46 and comprises the amino acid sequence of SEQ ID NO: 11 or 15.

54. The complex of any one of claims 47-53, wherein the second polypeptide comprises one VHH domain that binds NKp46.

55. The complex of any one of claims 35-54, wherein the first or the second polypeptide comprises at least one VHH domain that binds an antigen other than NKp46.

56. The complex of claim 55, wherein the first or the second polypeptide comprises at least one binding domain that binds TGFbeta receptor 1, TGFbeta receptor 2, or NKG2A.

57. The complex of claim 55 or claim 56, wherein the first or the second polypeptide comprises at least one binding domain that binds a tumor antigen.

58. The complex of any one of claims 35-57, wherein the first or the second polypeptide comprises at least one binding domain that binds an antigen selected from 1-92-LFA-3, 5T4, Alpha-4 integrin, Alpha-V integrin, alpha4beta1 integrin, alpha4beta7 integrin, AGR2, Anti-Lewis-Y, Apelin J receptor, APRIL, B7-H3, B7-H4, B7-H6, BAFF, BCMA, BTLA, C5 complement, C-242, CA9, CA19-9, (Lewis a), Carbonic anhydrase 9, CD2, CD3, CD6, CD9, CD11a, CD19, CD20, CD22, CD24, CD25, CD27, CD28, CD30, CD33, CD38, CD39, CD40, CD40L, CD41, CD44, CD44v6, CD47, CD51, CD52, CD56, CD64, CD70, CD71, CD73, CD74, CD80, CD81, CD86, CD95, CD117, CD123, CD125, CD132, (IL-2RG), CD133, CD137, CD138, CD166, CD172A, CD248, CDH6, CEACAM5 (CEA), CEACAM6 (NCA-90), CLAUDIN-3, CLAUDIN-4, cMet, Collagen, Cripto, CSFR, CSFR-1, CTLA-4, CTGF, CXCL10, CXCL13, CXCR1, CXCR2, CXCR4, CYR61, DL44, DLK1, DLL3, DLL4, DPP-4, DSG1, EDA, EDB, EGFR, EGFRviii, Endothelin B receptor (ETBR), ENPP3, EpCAM, EPHA2, EPHB2, ERBB3, F protein of RSV, FAP, FAS, FcRH5, FGF-2, FGF8, FGFR1, FGFR2, FGFR3, FGFR4, FLT-3, Folate receptor alpha (FRα), GAL3ST1, G-CSF, G-CSFR, GD2, GITR, GLUT1, GLUT4, GM-CSF, GM-CSFR, GP IIb/IIIa receptors, Gp130, GPIIB/IIIA, GPNMB, GPRC5D, GRP78, HAVCAR1, HER2/neu, HER3, HER4, HGF, hGH, HVEM, Hyaluronidase, ICOS, IFNalpha, 1FNbeta, IFNgamma, IgE, IgE Receptor (FceRI), IGF, IGF1R, IL1B, IL1R, IL2, IL1, IL12, IL12p40, IL-12R, IL-12Rbeta1, IL13, IL13R, IL15, IL17, IL18, IL21, IL23, IL23R, IL27/IL27R (wsx1), IL29, IL-31R, IL31/IL31R, IL2R, IL4, IL4R, IL6, IL6R, Insulin Receptor, Jagged Ligands, Jagged 1, Jagged 2, KISS1-R, LAG-3, LIF-R, Lewis X, LIGHT, LRP4, LRRC26, Ly6G6D, LyPD1, MCSP, Mesothelin, MICA, MICB, MRP4, MUC1, Mucin-16 (MUC16, CA-125), Na/K ATPase, NGF, Nicastrin, Notch Receptors, Notch 1, Notch 2, Notch 3, Notch 4, NOV, OSM-R, OX-40, PAR2, PDGF-AA, PDGF-BB, PDGFRalpha, PDGFRbeta, PD-1, PD-L1, PD-L2, Phosphatidyl-serine, P1GF, PSCA, PSMA, PSGR, RAAG12, RAGE, SLC44A4, Sphingosine 1 Phosphate, STEAP1, STEAP2, TAG-72, TAPA1, TEM-8, TGFbeta, TIGIT, TIM-3, TLR2, TLR4, TLR6, TLR7, TLR8, TL R9, TMEM31, TNFalpha, TNFR, TNFRS12A, TRAIL-R1, TRAIL-R2, Transferrin, Transferrin receptor, TRK-A, TRK-B, TROP-2 uPAR, VAP1, VCAM-1, VEGF, VEGF-A, VEGF-B, VEGF-C, VEGF-D, VEGFR1, VEGFR2, VEGFR3, VISTA, WISP-1, WISP-2, and WISP-3.

59. The complex of any one of claims 35-58, wherein at least one VHH domain, or each VHH domain, of the first or the second poly peptide is humanized.

60. The complex of any one of claims 35-59, wherein the first Fc region comprises at least one knob mutation and the second Fc region comprises at least one hole mutation, or wherein the first Fc region comprises at least one hole mutation and the second Fc region comprises at least one knob mutation.

61. The complex of claim 60, wherein the first or second Fc region comprises a T366W mutation and the other of the first or second Fc region comprises T366S, L368A, and Y407V mutations.

62. The complex of claim 61, wherein the Fc region comprising the T366S, L368A, and Y407V mutations further comprises a H435R or H435K mutation.

63. The complex of any one of claims 35-62, wherein the first polypeptide comprises the amino acid sequence of SEQ ID NO: 34, 40, 42, or 43.

64. The complex of any one of claims 35-63, wherein the second polypeptide comprises the amino acid sequence of SEQ ID NO: 33 or 41.

65. The complex of claim 63 or claim 64, wherein:
i) the first polypeptide comprises the amino acid sequence of SEQ ID NO: 34 and the second polypeptide comprises the amino acid sequence of SEQ ID NO: 33;
ii) the first polypeptide comprises the amino acid sequence of SEQ ID NO: 40 and the second polypeptide comprises the amino acid sequence of SEQ ID NO: 33;
iii) the first polypeptide comprises the amino acid sequence of SEQ ID NO: 42 and the second polypeptide comprises the amino acid sequence of SEQ ID NO: 41; or
iv) the first polypeptide comprises the amino acid sequence of SEQ ID NO: 43 and the second polypeptide comprises the amino acid sequence of SEQ ID NO: 41.

66. The complex of any one of claims 35-65, wherein the complex forms under physiological conditions.

67. An immunoconjugate comprising the polypeptide or complex of any one of claims 1-66 and a cytotoxic agent.

68. The immunoconjugate of claim 67, wherein the cytotoxic agent is selected from a calicheamicin, an auristatin, a dolastatin, a tubulicin, a maytansinoid, a cryptophycin, a duocarmycin, an esperamicin, a pyrrolobenzodiazepine, and an enediyne antibiotic.

69. The immunoconjugate of claim 67 or 68, wherein the immunoconjugate comprises a complex of any one of claims 35-66, and wherein the first or the second polypeptide comprises at least one binding domain that binds CD3, T-cell receptor (TCR) α, TCRβ, CD28, CD16, CD32A, CD64, CD89, or NKG2D.

70. A pharmaceutical composition comprising the polypeptide or complex of any one of claims 1-66 or the immunoconjugate of any one of claims 67-69, and a pharmaceutically acceptable carrier.

71. An isolated nucleic acid that encodes the polypeptide of any one of claims 1-34.

72. A vector comprising the nucleic acid of claim 71.

73. A host cell comprising the nucleic acid of claim 71 or the vector of claim 72.

74. A host cell that expresses the polypeptide of any one of claims 1-34.

75. A method of producing the polypeptide of any one of claims 1-34, comprising incubating the host cell of claim 73 or claim 74 under conditions suitable for expression of the polypeptide.

76. The method of claim 75, further comprising isolating the polypeptide.

77. An isolated nucleic acid that encodes the first polypeptide and the second polypeptide of the complex of any one of claims 35-66.

78. A vector that comprises the isolated nucleic acid of claim 77.

79. A host cell comprising the nucleic acid of claim 77 or the vector of claim 78.

80. A host cell that expresses the complex of any one of claims 35-66.

81. A method of producing the complex of any one of claims 35-66, comprising incubating the host cell of claim 73 or claim 74 under conditions suitable for expression of the complex.

82. The method of claim 81, further comprising isolating the complex.

83. A method of treating cancer comprising administering to a subject with cancer a pharmaceutically effective amount of the polypeptide or complex of any one of claims 1-66, the immunoconjugate of any one of claims 67-69, or the pharmaceutical composition of claim 70.

84. The method of claim 83, wherein the cancer is selected from basal cell carcinoma, biliary tract cancer; bladder cancer; bone cancer; brain and central nervous system cancer; breast cancer; cancer of the peritoneum, cervical cancer; choriocarcinoma, colon and rectum cancer; connective tissue cancer, cancer of the digestive system; endometrial cancer; esophageal cancer; eye cancer; cancer of the head and neck; gastric cancer (including gastrointestinal cancer); glioblastoma; hepatic carcinoma, hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx), ovarian cancer, pancreatic cancer; prostate cancer; retinoblastoma, rhabdomyosarcoma; rectal cancer; cancer of the respiratory system; salivary gland carcinoma, sarcoma; skin cancer; squamous cell cancer; stomach cancer; testicular cancer; thyroid cancer; uterine or endometrial cancer; cancer of the urinary system; vulval cancer, lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); acute myeloid leukemia (AML); Hairy cell leukemia; and chronic myeloblastic leukemia.

85. The method of claim 83 or claim 84, further comprising administering an additional therapeutic agent.

86. The method of claim 85, wherein the additional therapeutic agent is an anti-cancer agent.

87. The method of claim 86, wherein the anti-cancer agent is selected from a chemotherapeutic agent, an anti-cancer biologic, radiation therapy, CAR-T therapy, and an oncolytic virus.

88. The method of claim 87, wherein the additional therapeutic agent is an anti-cancer biologic.

89. The method of claim 88, wherein the anti-cancer biologic is an agent that inhibits PD-1 and/or PD-L1.

90. The method of claim 88, wherein the anti-cancer biologic is an agent that inhibits VISTA, gpNMB, B7H3, B7H4, HHLA2, CTLA4, or TIGIT.

91. The method of any one of claims 86-90, wherein the anti-cancer agent is an antibody.

92. The method of claim 91, wherein the antibody comprises a binding domain that binds a tumor antigen.

93. The method of claim 88, wherein the anti-cancer biologic is a cytokine.

94. The method of claim 87, wherein the anti-cancer agent is CAR-T therapy.

95. The method of claim 87, wherein the anti-cancer agent is an oncolytic virus.

96. The method of any one of claims 83-95, further comprising tumor resection and/or radiation therapy.

97. A method of redirecting a natural killer mediated cytotoxic response to a cancer cell comprising administering to a subject with cancer a pharmaceutically effective amount of the polypeptide or complex of any one of claims 1-66, the immunoconjugate of any one of claims 67-69, or the pharmaceutical composition of claim 70.

98. A method of treating an infectious disease comprising administering to a subject with an infectious disease a pharmaceutically effective amount of the polypeptide or complex of any one of claims 1-66, the immunoconjugate of any one of claims 67-69, or the pharmaceutical composition of claim 70.

99. The method of claim 98, wherein the infectious disease is a bacterial, viral, or fungal infection.

100. The method of claim 98 or 99, further comprising administering an additional therapeutic agent.

101. The method of claim 100, wherein the additional therapeutic agent is an antibiotic, an anti-viral agent, or an anti-fungal agent.

102. A method of redirecting a natural killer mediated cytotoxic response to a pathogen comprising administering to a subject with an infectious disease caused by the pathogen a pharmaceutically effective amount of the polypeptide or complex of any one of claims 1-66, the immunoconjugate of any one of claims 67-69, or the pharmaceutical composition of claim 70.

103. The method of any one of claims 98-102, wherein the polypeptide, complex, or immunoconjugate comprises at least one binding domain that binds an antigen expressed by the pathogen.
